(19) United States
(12) Patent Application Publication
(10) Pub. No.: US 2024/0002853 A1
(43) Pub. Date: Jan. 4, 2024

(54) NUCLEIC ACID DUPLEXES

(71) Applicant: ALPHA ANOMERIC SAS, Paris (FR)

(72) Inventors: Wolfgang RENNER, Bözberg (CH); Damien EVÉQUOZ, Vétroz (CH)

(21) Appl. No.: 18/253,853

(22) PCT Filed: Nov. 22, 2021
(86) PCT No.: PCT/EP2021/082539

§ 371 Date: May 22, 2023

(30) Foreign Application Priority Data
Nov. 23, 2020 (EP) 20209313.4

(51) Int. Cl. C12N 15/113; A61P 21/00

(52) U.S. Cl. CPC C12N 15/113; A61P 21/00; C12N 2310/32; C12N 2310/315; C12N 2310/341; C12N 2310/11

(57) ABSTRACT

The present invention relates to a compound comprising a first oligomeric compound and a second oligomeric compound, wherein the first oligomeric compound comprises a first oligonucleotide and said second oligomeric compound comprises a second oligonucleotide, wherein said first oligonucleotide has a nucleobase sequence that is complementary to a nucleic acid target, and wherein preferably said first oligonucleotide is an antisense oligonucleotide; and wherein said second oligonucleotide has a nucleobase sequence that is complementary to the nucleobase sequence of the first oligonucleotide; and wherein the affinity of said first oligonucleotide to said second oligonucleotide is lower than the affinity of said first oligonucleotide to the fully complementary unmodified RNA oligonucleotide of said first oligonucleotide; or wherein the biostability of said second oligonucleotide is lower than the biostability of said first oligonucleotide.

The present invention relates to antisense compounds and duplexes as well pharmaceutical compositions comprising the same, and their uses for the treatment, amelioration and/or prevention of diseases.

### RELATED ART

Oligonucleotides including antisense oligonucleotides (AON) can be used to modulate gene expression via several processes providing potential as therapeutics for a plurality of indications. However, despite recent technological advances with several oligonucleotide drugs gaining regulatory approval, a major obstacle preventing widespread usage of oligonucleotide therapeutics is still—beside to ensure potency and activity of the AON—the difficulty in achieving efficient delivery to target organs and tissues other than the liver to ensure overall sufficient efficacy and the acute toxicity reported of the phosphorothioate linkages still overwhelmingly used in AONs (Roberts, T. C. et al. (2020) Nat. Rev. Drug. Discov. 19(10):673-694; Iannitti, et al. (2014) Curr. Drug Targets 15:663-73).

Several different strategies have been investigated for their ability to improve the cellular delivery of oligonucleotides and AON's including chemical modifications such as backbone, sugar and nucleobase modifications, alternative oligonucleotide chemistries as well as bio-conjugations with antibodies, cell-penetrating peptides, lipids or sugars such as N-acetylgalactosamine (GalNAc). However, only a few demonstrated efficacy and still mostly target hepatocytes (Cuellar T. L. et al. (2015) Nucleic Acids Res 43(2): Benizri S. et al. (2019) Bioconjugate Chem. 30:366-383; 1189-1203; Gait M. J. et al. Nucleic Acid. Ther. (2019) 29(1):1-12; Roberts, T. C. et al. (2020) Nat. Rev. Drug. Discov. 19(10):673-694; Taylor R. E. et al. (2020) Pharmaceutics 12:225, all aforementioned documents are incorporated herein by reference in their entirety).

A further approach used a DNA/locked nucleotide acid (LNA) gapmer as the AON duplexed with a complementary RNA strand to which α-tocopherol is conjugated (Toc-HDO). The α-tocopherol conjugated to the complementary RNA strand of the heteroduplex oligonucleotide (HDO) has been described to improve the delivery of the Toc-HDO to the liver. Toc-HDO was found to be more potent at reducing the expression of the targeted mRNA in liver compared with the parent single-stranded gapmer AON (Nishina K. et al. (2015) Nature Communications, 6:7969; Asami Y. et al. (2016) Drug Discoveries & Therapeutics 10(5):256-262). The same Toc-HDO was found to efficiently reduce the expression of organic anion transporter 3 (OAT3) gene in brain microvascular endothelial cells (BMECs) in mice (Kuwahara H. et al. (2018) Science Reports 8:4377). Duplexed AONs have further been described in Yoshioka K. et al (2019) Nucleic Acids Research, 47(14):7321-7332; Mon S. S. L. et al. (2020) FEBS Letters 594:1413-1423; WO2013/089283, WO2014/132671, WO2014/192310, WO2014/203518, WO2016/6077704, WO2017/053999, WO2017/068790, WO2017/068791, WO2018/056442, WO2018/062510, WO2019/004420, WO2019/118916, WO2019/177061, WO2019/181946, WO2019/182109, WO2020/171149; all aforementioned documents are incorporated herein by reference in their entirety.

However, there is still a need to facilitate uptake and distribution of antisense compounds, and thus the potency and efficacy of antisense compound particularly in extrahepatic tissues as well as to decrease potential side effects and toxicity of prior art approaches and, thus, leading to improvements not only in efficacy but also in patient compliance and/or safety.

### SUMMARY OF THE INVENTION

We have surprisingly found that the compounds of the present invention that contain a first nucleic acid strand capable of interacting with a nucleic acid target such as an RNA target and a second nucleic acid strand complementary to the first nucleic acid strand show markedly improved activity. Such improved activity for the inventive compounds comprising AONs has been observed, in particular, for exon skipping activity when compared to the corresponding single-stranded AONs not only in gymnotic experiment in vitro, but also when injected intra-muscularly to mice in vivo. Without being bound, it is believed that the second nucleic acid strands of the invention have the purpose of altering the secondary structure of the first nucleic acid strand such that the resulting duplex mimics the shape of double stranded DNA. A possible explanation for the observed beneficial effect is that nucleic acids that are organized into a helical and double-stranded secondary structure which more efficiently interact with cell surface receptors than do single stranded oligonucleotides that are present in a random coil structure. A second nucleic acid strand should therefore be present during the transport phase from the initial administration, distribution via the blood stream, target cell binding and uptake into said target cells and potentially even transport into the nucleus. Thereafter the secondary strand should dissociate from the first nucleic acid strand or, alternatively be degraded, in a manner that the first antisense oligonucleotide is free to interact with the target nucleic acid such as the RNA.

The first oligonucleotide can be composed of various backbone chemistries such as abcDNA, tcDNA, MOE, 2′OMe, PMO, LNA, 2′-FANA, 2′F-RNA, any 2′OR modified chemistry, LNA or LNA analogs, ceNA, HNA or FHNA (Juliano R L et al., Acc Chem Res. 2012; 45(7):1067-1076, Anosova et al, Nucleic Acids Research, 2016, 44(3):1007-1021, WO 2019/215333). Furthermore various backbone modifications have been described in the literature that are applicable for the present invention. In preferred embodiments, the first oligonucleotide is essentially free of phosphorothioate internucleosidic linkages, which have shown to often cause dose limiting toxicity. Essentially free means that the first antisense oligonucleotide carries five, four, or three phosphorothioate linkages, better two or one, or ideally is completely free of phosphorothioate linkages.

Furthermore, the first oligonucleotide can carry a ligand, e.g. a ligand that prevents rapid clearance like a fatty acid or cholesterol ligand. Alternatively the targeting ligand can be a vitamin, a sugar like GalNAc, a peptide, an aptamer, a nanoparticle, a radioligand, an antibody or a fragment thereof, a darpin, a centyrin or any other ligand that targets a desired receptor within the body.

The second complementary oligonucleotide can be composed of various backbone chemistries, such as DNA, abcDNA, MOE, FANA, PMO, 2′OMe, LNA or any other suitable chemistry such as indicated below and herein. Since the second oligonucleotide that is associated with the first oligonucleotide via Watson-Crick base pairing has to be replaced with the target RNA upon entry into the nucleus by the latest, the second oligonucleotide has to be constructed in such a way that this replacement can occur. Second oligonucleotides of the present invention are therefore either less stable than the first oligonucleotide, i.e. are degraded more rapidly in serum than the first oligonucleotide, or have a lower affinity to the first oligonucleotide than said first oligonucleotide to the nucleic acid target such as the target RNA, as measured e.g. and typically and preferably by the Tm. In a preferred embodiment, both of said features are included.

A suitable second oligonucleotide therefore is composed of a backbone chemistry that is susceptible to nuclease degradation to a certain extent. Some level of stability is nonetheless required. A second oligonucleotide should have a reasonable long half-life in serum of at least 30 minutes, better more than one hour. Some backbone chemistries have an inherently low nuclease stability therefore require some level of stabilization. DNA, e.g. is very rapidly degraded and therefore may require some level of stabilization by the inclusion of one or more stabilizing residues of a different backbone chemistry such as MOE, 2′OMe, abcDNA or others such as LNA, tcDNA, TNA, ceNA, HNA or FHNA. Alternatively, internucleosidic linkages other than phosphodiester linkages such as phosphorothioate, phosphorodithioate can be applied. The second oligonucleotide should, in accordance with the invention have a lower stability than the first oligonucleotide. This can be achieved by replacing residues of the second oligonucleotide chemistry with residues of a less stable backbone chemistry.

The second oligonucleotide can alternatively have an affinity to the first oligonucleotide that is lower than the affinity of the first oligonucleotide with its target nucleic acid such as an RNA. This can be achieved by several means. The length of the second oligonucleotide can be selected accordingly. The shortening can occur on both ends of the second oligonucleotide, i.e. the 3′ end the 5′ end or the 7′ end whichever occurs. Alternatively a lowering of the Tm can be achieved by the insertion of one or more mismatches into the second oligonucleotide. A mismatch will lower the Tm and incorporating mismatches is thus a way to tailor the Tm in accordance with the present invention. Alternatively both of the above instruments, i.e shortening of the second oligonucleotide, and introducing mismatches into the second oligonucleotide can be combined. A second oligonucleotide can both be less stable in serum and have a lower Tm as compared to the first antisense oligonucleotide. Second oligonucleotides of the present invention typically and preferably do not carry an additional ligand, as their main purpose is to change the secondary structure of the first antisense oligonucleotide.

In a first aspect, the present invention provides for a compound comprising a first oligomeric compound and a second oligomeric compound, wherein the first oligomeric compound comprises a first oligonucleotide and said second oligomeric compound comprises a second oligonucleotide, wherein
said first oligonucleotide comprises at least one abc-DNA nucleoside, and wherein said first oligonucleotide has a nucleobase sequence that is complementary to a nucleic acid target, and wherein preferably said first oligonucleotide is an antisense oligonucleotide; and wherein
said second oligonucleotide has a nucleobase sequence that is complementary to the nucleobase sequence of the first oligonucleotide.

In a further aspect, the present invention provides for a compound comprising a first oligomeric compound and a second oligomeric compound, wherein the first oligomeric compound comprises a first oligonucleotide and said second oligomeric compound comprises a second oligonucleotide, and wherein
said first oligonucleotide has a nucleobase sequence that is complementary to a nucleic acid target, and wherein preferably said first oligonucleotide is an antisense oligonucleotide; and wherein said second oligonucleotide has a nucleobase sequence that is complementary to the nucleobase sequence of the first oligonucleotide; and wherein
the affinity of said first oligonucleotide to said second oligonucleotide is lower than the affinity of said first oligonucleotide to the fully complementary unmodified RNA oligonucleotide of said first oligonucleotide, wherein preferably said lower affinity corresponds to a lower melting temperature Tm of the duplex of said first and second oligonucleotide as compared to the melting temperature Tm of the duplex of said first oligonucleotide and the fully complementary unmodified RNA oligonucleotide of said first oligonucleotide, and wherein further preferably said melting temperatures Tm are determined as described in Example 2;
or wherein
the biostability of said second oligonucleotide is lower than the biostability of said first oligonucleotide, wherein preferably said lower biostability corresponds to a lower half-life stability in serum, preferably in mouse serum, wherein further preferably said lower biostability corresponds to a lower half-life stability in mouse serum as determined by AEX-chromatography after denaturating the duplex of said first and said second oligonucleotide, and again further preferably said lower biostability corresponds to a lower half-life stability in mouse serum as determined by AEX-chromatography after denaturating the duplex of said first and said second oligonucleotide as described in Example 3.

In a preferred embodiment, the affinity of said first oligonucleotide to said second oligonucleotide is lower than the affinity of said first oligonucleotide to the fully complementary unmodified RNA oligonucleotide of said first oligonucleotide; and the biostability of said second oligonucleotide is lower than the biostability of said first oligonucleotide. In a further preferred embodiment, the affinity of said first oligonucleotide to said second oligonucleotide is lower than the affinity of said first oligonucleotide to the fully complementary unmodified RNA oligonucleotide of said first oligonucleotide, wherein said lower affinity corresponds to a lower melting temperature Tm of the duplex of said first and second oligonucleotide as compared to the melting temperature Tm of the duplex of said first oligonucleotide and the fully complementary unmodified RNA oligonucleotide of said first oligonucleotide. In a further preferred embodiment, said melting temperatures Tm are determined as described in Example 2. In a further preferred embodiment, the biostability of said second oligonucleotide is lower than the biostability of said first oligonucleotide, wherein said lower biostability corresponds to a lower half-life stability in serum, preferably in mouse serum. In a further preferred embodiment, the biostability of said second oligonucleotide is lower than the biostability of said first oligonucleotide, wherein said lower biostability corresponds to a lower half-life stability in mouse serum. In a further preferred embodiment, the lower biostability corresponds to a lower half-life stability in serum, preferably in mouse serum, as determined by AEX-chromatography after denaturating of the duplex of said first and said second oligonucleotide. In a further preferred embodiment, the biostability corresponds to a lower half-life stability in serum, preferably in mouse or human serum, further preferably in mouse serum as determined by AEX-chromatography after denaturating of the duplex of said first and said second oligonucleotide as described in Example 3.

In another aspect, the present invention provides for a compound comprising a first oligomeric compound and a second oligomeric compound, wherein the first oligomeric compound comprises a first oligonucleotide and said second oligomeric compound comprises a second oligonucleotide, wherein
said first oligonucleotide is a gapmer comprising at least one abc-DNA nucleoside, and wherein said first oligonucleotide has a nucleobase sequence that is complementary to a nucleic acid target, and wherein preferably said first oligonucleotide is an antisense oligonucleotide; and wherein
said second oligonucleotide has a nucleobase sequence that is complementary to the nucleobase sequence of the first oligonucleotide.

In another aspect, the present invention provides for a compound comprising a first oligomeric compound and a second oligomeric compound, wherein the first oligomeric compound comprises a first oligonucleotide and said second oligomeric compound comprises a second oligonucleotide, wherein
said first oligonucleotide has a nucleobase sequence that is complementary to a nucleic acid target, and wherein preferably said first oligonucleotide is an antisense oligonucleotide; and wherein said second oligonucleotide has a nucleobase sequence that is complementary to the nucleobase sequence of the first oligonucleotide; and wherein
the affinity of said first oligonucleotide to said second oligonucleotide is lower than the affinity of said first oligonucleotide to the fully complementary unmodified RNA oligonucleotide of said first oligonucleotide, wherein preferably said lower affinity corresponds to a lower melting temperature Tm of the duplex of said first and second oligonucleotide as compared to the melting temperature Tm of the duplex of said first oligonucleotide and the fully complementary unmodified RNA oligonucleotide of said first oligonucleotide, and wherein further preferably said melting temperatures Tm are determined as described in Example 2.

In a preferred embodiment, the affinity of said first oligonucleotide to said second oligonucleotide is lower than the affinity of said first oligonucleotide to the fully complementary unmodified RNA oligonucleotide of said first oligonucleotide, wherein said lower affinity corresponds to a lower melting temperature Tm of the duplex of said first and second oligonucleotide as compared to the melting temperature Tm of the duplex of said first oligonucleotide and the fully complementary unmodified RNA oligonucleotide of said first oligonucleotide. In a further preferred embodiment, said melting temperatures Tm are determined as described in Example 2.

In a further aspect, the present invention provides for a compound comprising a first oligomeric compound and a second oligomeric compound, wherein the first oligomeric compound comprises a first oligonucleotide and said second oligomeric compound comprises a second oligonucleotide, wherein
said first oligonucleotide has a nucleobase sequence that is complementary to a nucleic acid target, and wherein preferably said first oligonucleotide is an antisense oligonucleotide; and wherein said second oligonucleotide has a nucleobase sequence that is complementary to the nucleobase sequence of the first oligonucleotide; and wherein
the biostability of said second oligonucleotide is lower than the biostability of said first oligonucleotide, wherein preferably said lower biostability corresponds to a lower half-life stability in mouse serum, wherein further preferably said lower biostability corresponds to a lower half-life stability in mouse serum as determined by AEX-chromatography after denaturating the duplex of said first and said second oligonucleotide, and again further preferably said lower biostability corresponds to a lower half-life stability in mouse serum as determined by AEX-chromatography after denaturating the duplex of said first and said second oligonucleotide as described in Example 3.

In a further preferred embodiment, the biostability of said second oligonucleotide is lower than the biostability of said first oligonucleotide, wherein said lower biostability corresponds to a lower half-life stability in serum, preferably in mouse serum. In a further preferred embodiment, the biostability of said second oligonucleotide is lower than the biostability of said first oligonucleotide, wherein said lower biostability corresponds to a lower half-life stability in mouse serum. In a further preferred embodiment, the lower biostability corresponds to a lower half-life stability in serum, preferably in mouse serum, as determined by AEX-chromatography after denaturating the duplex of said first and said second oligonucleotide. In a further preferred embodiment, the biostability corresponds to a lower half-life stability in serum, preferably in mouse or human serum, further preferably in mouse serum as determined by AEX-chromatography after denaturating the duplex of said first and said second oligonucleotide as described in Example 3.

In again a further aspect, the present invention provides for a compound comprising a first oligomeric compound and a second oligomeric compound, wherein the first oligomeric compound comprises a first oligonucleotide and said second oligomeric compound comprises a second oligonucleotide, wherein
said first oligonucleotide has a nucleobase sequence that is complementary to a nucleic acid target, and wherein preferably said first oligonucleotide is an antisense oligonucleotide; and wherein said second oligonucleotide has a nucleobase sequence that is complementary to the nucleobase sequence of the first oligonucleotide.

### DESCRIPTION OF FIGURES

FIG. 1: Agarose gel for single stranded AONs, measuring wild type dystrophin mRNA and mouse exon 23 and exon 22+23 skipped products after transfection into C2C12 cells detected by nested RT-PCR.

FIG. 2: Agarose gel for inventive duplexes, measuring wild type dystrophin mRNA and mouse exon 23 and exon 22+23 skipped products after transfection into C2C12 cells detected by nested RT-PCR.

FIG. 3: Quantification of skipped products with an image processing program for transfection experiments. For each sample, skipping efficacy was measured by calculating the ratio between the total skipped products (mouse exon 23 and exon 22+23 skipped products) and the wild type product. For each single stranded AON and duplex in accordance with the present invention, the data is reported as a mean value and the error bar represents the standard deviation.

FIG. 4: Agarose gel for single stranded AONs, measuring wild type dystrophin mRNA and mouse exon 23 and exon 22+23 skipped products after gymnosis (naked delivery) into C2C12 cells detected by nested RT-PCR.

FIG. 5: Agarose gel for inventive duplexes, measuring wild type dystrophin mRNA and mouse exon 23 and exon 22+23 skipped products after gymnosis (naked delivery) into C2C12 cells detected by nested RT-PCR.

FIG. 6: Quantification of skipped products with an image processing program for gymnosis experiments. For each sample, skipping efficacy was measured by calculating the ratio between the total skipped products (mouse exon 23 and exon 22+23 skipped products) and the wild type product. For each single stranded AON and duplex in accordance with the present invention, the data is reported as a mean value and the error bar represents the standard deviation; a) Quantification for single stranded AONs and b) quantification for the inventive duplexes.

FIG. 7: Agarose gel for single stranded AONs and inventive duplexes, measuring wild type dystrophin mRNA and mouse exon 23 and exon 22+23 skipped products after intra-muscular injections in mdx mice detected by nested RT-PCR.

FIG. 8: Quantification of skipped products with a lab-on-a-chip for intra-muscular injections in mdx mice. For each sample, skipping efficacy was measured by calculating the ratio between the total skipped product and the wild type product. For each single stranded AON and duplex in accordance with the present invention, the data is reported as a mean value and the error bar represents the standard deviation.

FIG. 9: Agarose gel for single stranded AONs and inventive duplexes comprising abcDNA, measuring wild type dystrophin mRNA and mouse exon 23 and exon 22+23 skipped products after gymnosis (naked delivery) into C2C12 cells detected by nested RT-PCR.

FIG. 10: Agarose gel for single stranded AONs and inventive duplexes comprising 2′-methoxyethyl nucleosides, measuring wild type dystrophin mRNA and mouse exon 23 and exon 22+23 skipped products after gymnosis (naked delivery) into C2C12 cells detected by nested RT-PCR.

FIG. 11: Agarose gel for single stranded AONs and inventive duplexes comprising phosphorodiamidate morpholino nucleic acid analogue nucleosides, measuring wild type dystrophin mRNA and mouse exon 23 and exon 22+23 skipped products after gymnosis (naked delivery) into KM155 cells detected by nested RT-PCR.

FIG. 12: Quantification of skipped products with an image processing program for gymnosis experiments. For each sample, skipping efficacy was measured by calculating the ratio between the total skipped products and the wild type product (mouse exon 23 and 22+23 skipped products in FIGS. 12A and 12B; human exon 23 and 22+23 in FIG. 12C). For each single stranded AON and duplex in accordance with the present invention, the data is reported as a mean value and the error bar represents the standard deviation. 12A: single-stranded AONs and inventive duplexes comprising abcDNA; 12B: single-stranded AONs and inventive duplexes comprising 2′-methoxyethyl nucleosides; 12C: single-stranded AONs and inventive duplexes comprising phosphorodiamidate morpholino nucleic acid analogue nucleosides.

FIG. 13: Agarose gel for single stranded AON and inventive duplexes comprising abcDNA, measuring wild type dystrophin mRNA and mouse exon 23 and exon 22+23 skipped products after intra-muscular injections in mdx mice detected by nested RT-PCR.

FIG. 14: Agarose gel for single stranded AON and inventive duplexes comprising 2′-methoxyethyl nucleosides, measuring wild type dystrophin mRNA and mouse exon 23 and exon 22+23 skipped products after intra-muscular injections in mdx mice detected by nested RT-PCR.

FIG. 15: Agarose gel for single stranded AON and inventive duplexes comprising phosphorodiamidate morpholino nucleosides, measuring wild type dystrophin mRNA and mouse exon 23 and exon 22+23 skipped products after intra-muscular injections in mdx mice detected by nested RT-PCR.

FIG. 16: Quantification of skipped products with a lab-on-a-chip for intra-muscular injections in mdx mice. For each sample, skipping efficacy was measured by calculating the ratio between the total skipped products and the wild type product. For each single stranded AON and duplex in accordance with the present invention, the data is reported as a mean value. FIG. 16A: abcDNA single-stranded AONs and inventive duplexes; FIG. 16B: 2′-methoxyethyl nucleoside single-stranded AONs and inventive duplexes; FIG. 16C: phosphorodiamidate morpholino nucleoside single-stranded AONs and inventive duplexes.

FIG. 17: In vitro kinetic measurements of RNase H activity of inventive gapmer duplexes. Parameters shown are initial reaction speed, Vₘₐₓ and Kₘ. 17A: DNA-gap1; 17B: abcDNA-gap1; 17C: abcDNA-gap2; 17D: abcDNA-gap3; 17E: DNA-gap2; 17F: abcDNA-gap4; 17G: abcDNA-gap5; 17H: MOE-gap.

### DETAILED DESCRIPTION OF THE INVENTION

Unless defined otherwise, all technical and scientific terms used herein have the same meanings as commonly understood by one of ordinary skill in the art to which this invention belongs. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

“Affinity” with reference between oligonucleotides such as the affinity of the first oligonucleotide to the second oligonucleotide or the affinity of the first oligonucleotide to the fully complementary unmodified RNA oligonucleotide of said first oligonucleotide, corresponds herein to and is measured by way of its UV monitored melting temperatures Tm of the respective duplexes such as the duplex of said first and second oligonucleotide and the duplex of said first oligonucleotide and the fully complementary unmodified RNA oligonucleotide of said first oligonucleotide. Such determination of the melting temperatures Tm is routine and is known to the skilled person in the art. The Tm values of duplexes such as DNA/DNA or DNA/RNA duplexes can be calculated by using the “Nearest Neighbor” methods, as described by Breslauer et al., 1986, Proc. Nat. Acad. Sci, 83:3746-50, but using the values for DNA as described by Sugimoto et al., 1996, Nucl. Acids Res. 24:4501-4505, and the values for RNA as described by Xia T. et al, 1998, Biochemistry 37:14719-14735. Alternatively, the Tm values can be determined by online tools using a similar methodology, such as OligoCalc as a web-accessible, client-based computational engine for reporting DNA and RNA single-stranded and double-stranded properties (Kibbe W A, Nucleic Acids Res. 2007, 35:W43-6). The impact of modified nucleotides can be determined by using the Tm of the corresponding natural duplex, and adding the increment corresponding to the modification as reported in the literature (Prakash T P, Chem Biodivers. 2011, 9:1616-1641). In a preferred embodiment, said melting temperatures Tm of such duplexes corresponding and being considered as the affinity in accordance with the present invention are determined as described in Example 2.

“Alpha anomeric bicyclo-DNA (“abc-DNA”) nucleoside” means a nucleoside containing a bicyclic sugar moiety and having the general structure shown in below. Abc-DNA nucleosides are linked together by its 7′-end or 7′-terminus and by its 5′-end or 5′-terminus, but not by way of its 3′-position. For the ease of reference herein, when it is referred to the 3′-end or 3′-terminus of a nucleoside, in particular in the context of a nucleotide, oligonucleotide or oligomeric compound, it should refer and include reference to the 7′-end or 7′-terminus if it is referred to and/or includes an abc-DNA. Vice versa, if it is referred herein to the 7′-end or 7′-terminus of an abcDNA it should refer to and/or include, if applicable, the reference to the 3′-end or 3′-terminus of other nucleosides or nucleotides.

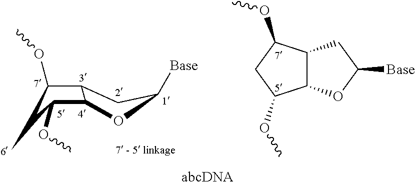

“Antisense activity”, means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound. In certain embodiments, antisense activity is a change in splicing of a pre-mRNA nucleic acid target. In certain embodiments, antisense activity is an increase in the amount or expression of a target nucleic acid or protein encoded by such target nucleic acid compared to target nucleic acid levels or target protein levels in the absence of the antisense compound. In certain embodiment, the antisense activity can be mediated by binding of the antisense compound to a target mRNA and inhibiting its translation by acting as a steric block. In certain embodiment, the antisense activity can be mediated by RNase H mediated decay of the pre-mRNA. In certain embodiment, the antisense activity can be mediated by binding of the antisense compound to a target miRNA and inhibiting its function. In certain embodiment, the antisense activity can be mediated by binding of the antisense compound to a target pre-mRNA and inducing alternative splicing, via inclusion or exclusion of one or several specific exons.

“Antisense compound” means a compound comprising an antisense oligonucleotide and optionally one or more additional features, such as a conjugate group or terminal group.

“Antisense oligonucleotide” means an oligonucleotide that (i) has a nucleobase sequence that is at least partially complementary to a target nucleic acid and that (ii) is capable of producing an antisense activity in a cell or animal.

“Bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

As used herein, a “bicyclic sugar moiety” comprises two interconnected ring systems, e.g. bicyclic nucleosides wherein the sugar moiety has a 2′-O—CH(alkyl)-4′ or 2′-O—CH₂-4′ group, locked nucleic acid (LNA), xylo-LNA, alpha-L-LNA, beta-D-LNA, cEt (2′-O,4′-C constrained ethyl) LNA, cMOEt (2′-O,4′-C constrained methoxyethyl) LNA, ethylene-bridged nucleic acid, or abc-DNA.

“Biostability” means the ability of a compound to remain intact in biological media or in vivo. The biostability of oligonucleotides can be measured and compared by incubating the oligonucleotide in serum, typically and preferably in mice or human serum, and measuring the percentage of intact product remaining at different time points.

“Complementary” in reference to an oligonucleotide means that at least 70% of the nucleobases of such oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions, for oligonucleotides forming antiparallel duplexes, or in similar direction, for oligonucleotides forming parallel duplexes. Complementary nucleobases means nucleobases that are capable of forming hydrogen bonds with one another. Complementary nucleobase pairs include, but unless otherwise specific are not limited to, adenine (A) and thymine (T), adenine (A) and uracil (U), cytosine (C) and guanine (G), 5-methyl cytosine (^{m}C) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that such oligonucleotides are complementary to another oligonucleotide or nucleic acid at each nucleoside of the oligonucleotide.

“Conjugate group” means a group of atoms that is directly or indirectly attached to an oligonucleotide. Conjugate groups include a conjugate moiety and a conjugate linker that attaches the conjugate moiety to the oligonucleotide.

“Conjugate linker” means a group of atoms comprising at least one bond that connects a conjugate moiety to an oligonucleotide.

“Conjugate moiety” means a group of atoms that is attached to an oligonucleotide via a conjugate linker.

“Contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

“Duplex” means two oligomeric compounds that are paired. In certain embodiments, the two oligomeric compounds are paired via hybridization of complementary nucleobases.

“Extra-hepatic cell type” means a cell type that is not a hepatocyte.

“Extra-hepatic nucleic acid target” means a target nucleic acid that is expressed in tissues other than liver. In certain embodiments, extra-hepatic nucleic acid targets are not expressed in the liver or not expressed in the liver at a significant level. In certain embodiments, extra-hepatic nucleic acid targets are expressed outside the liver and also in the liver.

“Extra-hepatic tissue” means a tissue other than liver.

“Fully modified” in reference to a modified oligonucleotide means a modified oligonucleotide in which each sugar moiety is modified. “Uniformly modified” in reference to a modified oligonucleotide means a fully modified oligonucleotide in which each sugar moiety is the same. For example, the nucleosides of a uniformly modified oligonucleotide can each have a 2′-MOE modification or are all abc-DNA nucleosides, but still different nucleobase modifications.

“Gapmer” means an antisense oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.”

“Hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

“Inhibiting the expression or activity” refers to a reduction or blockade of the expression or activity relative to the expression of activity in an untreated or control sample and does not necessarily indicate a total elimination of expression or activity.

“Internucleoside linkage” means a group or bond that forms a covalent linkage between adjacent nucleosides in an oligonucleotide. As used herein “modified internucleoside linkage” means any internucleoside linkage other than a naturally occurring, phosphate internucleoside linkage contain a phosphodiester (“PO”). Non-phosphate linkages are referred to herein as modified internucleoside linkages. “Phosphorothioate linkage” means a modified phosphate linkage in which one of the non-bridging oxygen atoms is replaced with a sulfur atom (“PS”). A phosphorothioate internucleoside linkage is a modified internucleoside linkage.

“Linked nucleosides” are nucleosides that are connected in a continuous sequence (i.e. no additional nucleosides are present between those that are linked).

“Lipid group” in reference to a chemical group means a group of atoms that is more soluble in lipids or organic solvents than in water and/or has a higher affinity for lipids than for water. In certain embodiments, lipid groups comprise or are derived from a lipid. As used herein “lipid” means a molecule that is not soluble in water or is less soluble in water than in organic solvents. In certain embodiments, compounds of the present invention comprise lipids selected from saturated or unsaturated fatty acids, steroids, lipid soluble vitamins, phospholipids, sphingolipids, hydrocarbons, mono-, di-, and tri-glycerides, and synthetic derivatives thereof.

“Mismatch” or “non-complementary” means a nucleobase of the first oligonucleotide that is not complementary with the corresponding nucleobase of the second oligonucleotide or target nucleic acid when the first and second oligomeric compound or first oligomeric compound and target nucleic acid are aligned.

“MOE” means methoxyethyl. “2′-MOE” means a —OCH₂CH₂OCH₃ group at the 2′ position of a furanosyl ring.

“Motif’ means the pattern of unmodified and/or modified sugar moieties, nucleobases, and/or internucleoside linkages, in an oligonucleotide. In certain embodiments, the first and/or second oligonucleotide comprises one or more unmodified or modified nucleoside comprising an unmodified or a modified sugar. In certain embodiments, the first and/or second oligonucleotide comprises one or more unmodified or modified nucleosides comprising an unmodified or modified nucleobase. In certain embodiments, the first and/or second oligonucleotide comprises one or more unmodified or modified internucleoside linkage. In such embodiments, the modified, unmodified, and differently modified sugar moieties, nucleobases, and/or internucleoside linkages of the first and/or second oligonucleotide define a pattern or motif. The patterns of sugar moieties, nucleobases, and internucleoside linkages are each independent of one another. Thus, the first and/or second oligonucleotide may be independently described by its sugar motif, nucleobase motif and/or internucleoside linkage motif.

“Naturally occurring” means found in nature.

“Nucleobase” means an unmodified nucleobase or a modified nucleobase. As used herein a “an unmodified nucleobase” is adenine (A), thymine (T), cytosine (C), uracil (U), and guanine (G). As used herein, a “modified nucleobase” is a group of atoms other than unmodified A, T, C, U, or G capable of pairing with at least one unmodified nucleobase. A universal base is a modified nucleobase that can pair with any one of the five unmodified nucleobases. As used herein, “nucleobase sequence” means the order of contiguous nucleobases in a nucleic acid or oligonucleotide independent of any sugar or internucleoside linkage modification.

“Nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides, which lack a nucleobase. As used herein, “unmodified DNA nucleoside” or “2′-deoxy nucleoside” means a nucleoside comprising a 2-deoxyribose sugar moiety and typically and preferably an unmodified nucleobase. As used herein, “2′-MOE nucleoside” means a nucleoside comprising a ribose sugar moiety having a MOE attached to the pentose ring in the 2′-position. As used herein, “2′-OMe nucleoside” means a nucleoside comprising a ribose sugar moiety having a O-methyl attached to the pentose ring in the 2′-position.

“Oligomeric compound” means a compound consisting of an oligonucleotide and optionally one or more additional features, such as a conjugate group or terminal group.

“Oligonucleotide” means a strand of linked nucleosides connected via internucleoside linkages, wherein each nucleoside and internucleoside linkage may be modified or unmodified. Unless otherwise indicated, oligonucleotides consist of 6-50 linked nucleosides. As used herein, “modified oligonucleotide” means an oligonucleotide, wherein at least one nucleoside is modified. As used herein, “unmodified oligonucleotide” means an oligonucleotide that does not comprise any nucleoside modifications. As used herein, “unmodified RNA oligonucleotide” means an oligonucleotide wherein each nucleoside thereof has an unmodified sugar moiety as found in RNA (an “unmodified RNA sugar moiety”) which are all linked by PO internucleoside linkages, and wherein all nucleobases are unmodified nucleobases.

An “overhanging” nucleotide refers to a nucleotide or nucleotide region in a first oligonucleotide strand wherein the 5′ end of the first oligonucleotide strand extends beyond the 3′ end of the second oligonucleotide strand, and/or wherein the 3′ end of the first oligonucleotide strand extends beyond the 5′ end of the second oligonucleotide strand when the first and second oligonucleotide strands are annealed to each other to form a duplex structure, namely a nucleotide region protruding from the duplex structure. The overhang region is adjacent to the complementary region.

“Pharmaceutically acceptable carrier or diluent” means any substance suitable for use in administering to an animal. Certain such carriers enable pharmaceutical compositions to be formulated as, for example, tablets, pills, dragees, capsules, liquids, gels, symps, slurries, suspension and lozenges for the oral ingestion by a subject. In certain embodiments, a pharmaceutically acceptable carrier or diluent is sterile water; sterile saline; or sterile buffer solution.

“Pharmaceutically acceptable salts” means physiologically and pharmaceutically acceptable salts of compounds, such as oligomeric compounds, i.e., salts that retain the desired biological activity of the parent compound and do not impart undesired toxicological effects thereto.

“Pharmaceutical composition” means a mixture of substances suitable for administering to a subject. For example, a pharmaceutical composition may comprise an antisense compound or a duplex, and a sterile aqueous solution.

“Phosphorus moiety” means a group of atoms comprising a phosphorus atom. In certain embodiments, a phosphorus moiety comprises a mono-, di-, or tri-phosphate, or phosphorothioate.

“Single-stranded” in reference to an oligomeric compound means such a compound that is not paired with a second oligomeric compound to form a duplex. “Self-complementary” in reference to an oligonucleotide means an oligonucleotide that at least partially hybridizes to itself. A compound consisting of one oligomeric compound, wherein the oligonucleotide of the oligomeric compound is self-complementary, is a single-stranded compound. A single-stranded antisense or oligomeric compound may be capable of binding to a complementary oligomeric compound to form a duplex, in which case it would no longer be single-stranded.

“Sugar moiety” means an unmodified sugar moiety or a modified sugar moiety. As used herein, “unmodified sugar moiety” means a 2′-OH(H) furanosyl moiety, as found in RNA (an “unmodified RNA sugar moiety”), or a 2′-H(H) moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate. As used herein, modified furanosyl sugar moiety means a furanosyl sugar comprising a non-hydrogen substituent in place of at least one hydrogen of an unmodified sugar moiety. In certain embodiments, a modified furanosyl sugar moiety is a 2′-substituted sugar moiety. Such modified furanosyl sugar moieties include bicyclic sugars and non-bicyclic sugars. As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

“Target nucleic acid” means a naturally occurring, identified nucleic acid. In certain embodiments, target nucleic acids are endogenous cellular nucleic acids, including, but not limited to RNA transcripts, pre-mRNA, mRNA, long non-coding RNA, small RNAs like ribosomal RNA (rRNA), transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). In certain embodiments, target nucleic acids are viral nucleic acids. In certain embodiments, target nucleic acids are nucleic acids that an antisense compound is designed to affect. “Target region” means a portion of a target nucleic acid to which an antisense compound is designed to hybridize.

“Muscle target” means a nucleic acid transcript for which there is some desired therapeutic benefit from modulating the amount or activity of the nucleic acid transcript in muscle tissue. Muscle tissue includes, but is not limited to smooth muscle tissue and skeletal muscle tissue. For example, a given nucleic acid transcript may be expressed in multiple tissues, however one or more therapeutic benefit is achieved when the amount or activity of the target nucleic acid is modulated in muscle tissue.

“Terminal group” means a chemical group or group of atoms that is covalently linked to a terminus of an oligonucleotide.

### Nucleosides

The first oligonucleotide and the second oligonucleotide of the present invention may be unmodified oligonucleotides (RNA or DNA) or may be modified oligonucleotides. Modified oligonucleotides, for example the first modified oligonucleotide or the second modified oligonucleotide, comprise at least one modification relative to unmodified RNA or DNA, i.e. comprise at least one modified nucleoside, and thus comprises a modified sugar moiety and/or a modified nucleobase. Modified nucleosides comprise a modified sugar moiety or a modified nucleobase or both a modified sugar moiety and a modified nucleobase.

In certain embodiments, modified nucleosides comprise a modified sugar moiety. Typical and preferred modified nucleosides are known to the skilled person in the art and have been described by Juliano R L et al (Acc Chem Res. 2012; 45(7):1067-1076, particular reference hereby is made to FIG. 2 thereof) and by Anosova et al (Nucleic Acids Research, 2016, 44(3):1007-1021, particular reference hereby is made to FIG. 2 thereof).

In certain embodiments, modified sugar moieties are non-bicyclic modified sugar moieties comprising a furanosyl ring with one or more acyclic substituent, including but not limited to substituents at the 2′, 4′, and/or 5′ positions. In certain embodiments one or more acyclic substituent of non-bicyclic modified sugar moieties is branched.

In certain embodiments, modified sugar moieties are non-bicyclic modified sugar moieties comprising a furanosyl ring with substituents at the 2′-positions. Typical and preferred examples of 2′-substituent groups suitable for non-bicyclic modified sugar moieties include but are not limited to 2′-F, 2′-OH, 2′-propargyl, 2′-O-propylamino, 2′-NH₂, 2′-OCH₃ (“OMe” or “O-methyl”), and 2′-O(CH₂)₂OCH₃ (“MOE”). Further 2′-substituent groups include allyl, azido, SH, CN, OCN, CF₃, OCF₃, alkynyl. Said 2′-substituent groups are known and have been described, inter alia, in U.S. Pat. Nos. 6,531,584; 5,859,221 and 6,005,087. In an embodiment, said modified nucleoside is 2′-modified-RNA nucleoside, wherein said 2′-modification is selected from the group consisting of 2′-F, 2′-OH, 2′-propargyl, 2′-O-propylamino, 2′-NH₂, 2′-OCH₃ (“OMe” or “O-methyl”), and 2′-O(CH₂)₂OCH₃ (“MOE”). Examples of 4′-substituent groups, 5′-substituent groups and 2′-5′-substituted groups suitable for non-bicyclic modified sugar moieties are known to the skilled person in the art and have been described in WO 2015/106128, WO 2008/101157 and US 2013/0203836. In certain embodiments, a 2′-substituted nucleoside or 2′-non-bicyclic modified nucleoside comprises a sugar moiety comprising a non-bridging 2′-substituent group selected from: F, OH, OCH₃, and OCH₂CH₂OCH₃.

Certain modified sugar moieties comprise a bridging sugar substituent that forms a second ring resulting in a bicyclic sugar moiety.

In certain such embodiments, the bicyclic sugar moiety comprises a bridge between the 4′ and the 3′ furanose ring atoms.

In preferred embodiments, said modified nucleosides comprise a modified sugar moiety, wherein said modified sugar moiety is a bicyclic sugar moiety derived from an abc-DNA nucleoside. In preferred embodiments, said modified nucleoside is an abc-DNA nucleoside. Alpha anomeric bicyclo-DNA nucleoside are known and have been described (Evéquoz D and Leumann C J, Chem Eur J, 2017, 23(33):7953-7968; WO2018/099946, WO2019/215333).

In certain such embodiments, the bicyclic sugar moiety comprises a bridge between the 4′ and the 2′ furanose ring atoms. Typical and preferred examples of such 4′ to 2′ bridging sugar substituents include but are not limited to 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (described, inter alia, in U.S. Pat. Nos. 7,399,845, 7,427,672, 7,569,686, 7,741,457, 8,022,193), U.S. Pat. Nos. 8,278,283, 8,278,425, 7,696,345, 8,124,745, 8,278,426 and by Zhou, et al, J. Org. Chem., 2009)

Further bicyclic sugar moieties are known and have been described by Freier et al (Nucleic Acids Research, 1997, 25(22), 4429-4443), Albaek et al (J. Org. Chem., 2006, 71, 7731-7740), Singh et al. (Chem. Commun., 1998, 4, 455-456; J. Org. Chem., 1998, 63, 10035-10039), Koshkin et al (Tetrahedron, 1998, 54, 3607-3630), Kumar et al (Bioorg. Med. Chem. Lett., 1998, 8, 2219-2222), Srivastava et al. (J. Am. Chem. Soc., 2017, 129, 8362-8379), and in U.S. Pat. Nos. 7,053,207, 6,268,490, 6,770,748, 6,794,499, 6,670,461, 7,034,133, 800,644, 8,034,909, 8,153,365, WO2004/106356, WO1999/014226, WO2007/134181, U.S. Pat. Nos. 7,547,684, 7,666,854, 8,088,746, 7,750,131, 8,030,467, 8,268,980, 8,546,556, 8,530,640, 9,012,421, 8,501,805.

In certain embodiments, bicyclic sugar moieties and nucleosides incorporating such bicyclic sugar moieties are further defined by isomeric configuration. For example, an LNA nucleoside may be in the α-L configuration or in the β-D configuration (Frieden et al, Nucleic Acids Research, 2003, 21, 6365-6372).

In certain embodiments, modified sugar moieties are sugar surrogates. In certain such embodiments, referring to typical examples, the oxygen atom of the sugar moiety is replaced, or certain sugar surrogates comprise a substitution at the 2′-position, or sugar surrogates comprise acyclic moieties, or a sugar surrogate comprises rings having other than 5 atoms such as a six-membered ring systems including tetrahydropyrans, or a sugar surrogate comprises rings having more than 5 atoms and more than one heteroatom, wherein all of said sugar surrogates may be further modified or substituted (U.S. Pat. Nos. 5,698,685, 5,166,315, 5,185,444, 5,034,506, 7,875,733, 7,939,677, 8,088,904, 8,440,803, 8,796,437, 9,005,906, WO2011/133876, Damha M J et al, J. Am. Chem. Soc. 1998, 120:12976-12977; Koshkin A A et al, J. Am. Chem. Soc. 1998, 120:13252-13253; Obika S et al, Tetrahedron Lett. 1998, 39:5401-5404; Schoning K et al, Science, 2000, 290:1347-1351; Leumann C J Bioorg & Med Chem 2002, 10:841-854; Nauwelaerts K et al, Nucleic Acids Res, 2005, 33:2452-2463; Wilds C J et al, Nucleic Acids Res. 2000, 28:3625-3635; Braasch et al, Biochemistry, 2002, 41:4503-4510; Schlegel M K et al, J. Am. Chem. Soc. 2008, 130:8158-8159; Zhang L er al, J. Am. Chem. Soc. 2005, 127:4174-4175; Kumar et al, Org Biomol Chem 2013, 11:5853-5865; Juliano R L et al Acc Chem Res. 2012; 45(7):1067-1076, particular reference hereby is made to FIG. 2 thereof), Anosova et al Nucleic Acids Research, 2016, 44(3):1007-1021, particular reference hereby is made to FIG. 2 thereof; Martin-Pintado N et al Nucleic Acids Research, 2012, 40(18):9329-9339).

In an embodiment, said modified nucleoside comprises a modified sugar moiety and sugar surrogate, respectively, wherein said modified sugar moiety and sugar surrogate is a cyclohexene nucleic acid (“CeNA”), a hexitol nucleic acid (“HNA”), mannitol nucleic acid (“MNA”), arabino nucleic acid (“ANA”) being the C2′-stereoisomer of RNA, and the 2′-fluoro-ANA analogue (“FANA”), a threose nucleic acid (TNA), a peptide nucleic acid (PNA), glycol nucleic acid (GNA), acyclic butyl nucleic acid, or a morpholino.

As used here, the term “morpholino” means a sugar surrogate having the following structure:

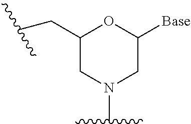

In certain embodiments, morpholinos may be modified, for example, by adding or altering various substituent groups from the above morpholino structure. Such sugar surrogates are herein referred to as “modified morpholinos”. See, by way of example, WO2008/036127, WO2011/150408 and US 2012/0065169.

As used herein, the term “PMO” (phosphorodiamidate morpholino oligomer) means an oligonucleotide consisting of uniformly modified nucleosides, wherein said modified nucleosides are morpholino nucleosides, and wherein each internucleoside linkage of said oligonucleotide is a phosphorodiamidate internucleoside linkage.

Many other bicyclic and tricyclic sugar and sugar surrogate ring systems are known in the art that can be used in modified nucleosides in accordance with the present invention.

In certain embodiments, modified sugar moieties are tricyclic modified sugar moieties comprised in tricyclic nucleosides (“tc-DNA”). Tricyclic nucleosides including their numerous modifications such as C(6′)-functionalized tc-DNA, 6′-fluoro-tc-DNA and 2′-fluoro-tc-DNA are known and have been described including its use as antisense compounds (Renneberg D and Leumann C J, J Am Chem Soc 2002, 124:5993-6002; Lietard J and Leumann C J, J Org Chem 2012, 77:4566-4577; WO2012/170347, WO 2013/135900, WO2014/140348, WO2018/055577, WO2018/193428).

In certain embodiments, the first and/or the second oligonucleotide comprise one or more type of modified sugar and/or unmodified sugar moiety arranged along said oligonucleotides or region thereof in a defined pattern or sugar motif. In certain instances, such sugar motifs include but are not limited to any of the sugar modifications discussed herein.

In certain embodiments, the first oligonucleotide and/or the second oligonucleotide, comprise or consist of a region having a gapmer motif, which comprises two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-5 nucleosides. In certain embodiments, the wings of a gapmer comprise 1-4 nucleosides. In certain embodiments, the wings of a gapmer comprise 1-3 nucleosides. In certain embodiments, the wings of a gapmer comprise 1-2 nucleosides. In certain embodiments, the wings of a gapmer comprise 2-5 nucleosides. In certain embodiments, the wings of a gapmer comprise 2-4 nucleosides. In certain embodiments, the nucleosides of a gapmer are all modified nucleosides. In certain embodiments, the gap of a gapmer comprises 7-12 nucleosides. In certain embodiments, each nucleoside of the gap of a gapmer is an unmodified 2′-deoxy nucleoside. In certain embodiments, the gapmer is a deoxy gapmer. In such embodiments, the nucleosides on the gap side of each wing/gap junction are unmodified 2′-deoxy nucleosides and the nucleosides on the wing sides of each wing/gap junction are modified nucleosides. In certain such embodiments, each nucleoside of the gap is an unmodified 2′-deoxy nucleoside.

In certain embodiments, the first oligonucleotide comprises or consists of a region having a fully modified sugar motif. In such embodiments, each nucleoside of the fully modified region of the first oligonucleotide comprises a modified sugar moiety. In certain such embodiments, each nucleoside to the entire first oligonucleotide comprises a modified sugar moiety. In certain embodiments, the first oligonucleotide comprises or consists of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified first oligonucleotide comprises the same abc-DNA derived sugar modification.

In certain embodiments, the second oligonucleotide comprises at least one 2′-MOE nucleoside. In certain embodiments, the second oligonucleotide comprises 2′-MOE and 2′-deoxy nucleosides. In certain such embodiments, the central region is comprised of 2′-deoxy nucleosides and the wing regions are modified nucleosides. In certain embodiments, the central region is comprised of 2′-deoxy nucleosides and the wing regions are 2′-MOE modified nucleosides. The internucleoside linkages of the second oligonucleotide may be modified or consist of phosphodiester internucleoside linkages. In certain embodiments, the internucleoside linkages of the second oligonucleotide follow a gapmer-like motif—phosphorothioate wings and phosphodiester internucleoside linkages in the center.

In a preferred embodiment, said first oligonucleotide comprises, preferably consists of, uniformly modified nucleosides. In a further preferred embodiment, said first oligonucleotide comprises, preferably consists of, uniformly modified nucleosides, wherein said modified nucleosides are alpha anomeric bicyclo-DNA (abc-DNA) nucleosides. In a further preferred embodiment, said first oligonucleotide comprises, preferably consists of, uniformly modified nucleosides, wherein said modified nucleosides are morpholino nucleosides. In a further preferred embodiment, said first oligonucleotide comprises, preferably consists of, uniformly modified nucleosides, wherein said modified nucleosides are 2′-MOE nucleosides.

In a further preferred embodiment, said second oligonucleotide comprises, preferably consists of, unmodified nucleosides and modified nucleosides, wherein said modified nucleosides are 2′-MOE nucleosides. In a further preferred embodiment, said second oligonucleotide essentially consists of unmodified nucleosides and at most four, preferably at most three, further preferably at most two or one modified nucleosides, wherein said modified nucleosides are 2′-MOE nucleosides. In a further preferred embodiment, said second oligonucleotide essentially consists of unmodified nucleosides and at most four, preferably at most three, further preferably at most two or one modified nucleosides, wherein said modified nucleosides are 2′-MOE nucleosides, and wherein said modified nucleosides are at the 5′-end/region/wing and/or at the 3′-end/region/wing of said second oligonucleotide. In a further preferred embodiment, said second oligonucleotide essentially consists of unmodified nucleosides and at most two or one modified nucleosides, wherein said modified nucleosides are 2′-MOE nucleosides, and wherein said modified nucleosides are the 3′-end/wing of said second oligonucleotide. In a further preferred embodiment, said second oligonucleotide comprises, preferably consists of, uniformly unmodified or uniformly modified nucleosides. In a further preferred embodiment, said second oligonucleotide comprises, preferably consists of, uniformly unmodified or uniformly modified nucleosides, wherein said modified nucleosides are selected from the group consisting of alpha anomeric bicyclo-DNA (abc-DNA) nucleosides, FANA nucleosides, morpholino nucleosides, 2′-MOE nucleosides, and 2′-OMe nucleosides.

In a further preferred embodiment, said second oligonucleotide comprises, preferably consists of, uniformly modified nucleosides, wherein said modified nucleosides are alpha anomeric bicyclo-DNA (abc-DNA) nucleosides. In a further preferred embodiment, said second oligonucleotide comprises, preferably consists of, uniformly modified nucleosides, wherein said modified nucleosides are FANA nucleosides. In a further preferred embodiment, said second oligonucleotide comprises, preferably consists of, uniformly modified nucleosides, wherein said modified nucleosides are morpholino nucleosides. In a further preferred embodiment, said second oligonucleotide comprises, preferably consists of, uniformly modified nucleosides, wherein said modified nucleosides are morpholino nucleosides, and wherein each internucleoside linkage of said second oligonucleotide is a phosphorodiamidate internucleoside linkage. In a further preferred embodiment, said second oligonucleotide is a phosphorodiamidate morpholino oligomer (PMO). In a further preferred embodiment, said second oligonucleotide comprises, preferably consists of, uniformly modified nucleosides, wherein said modified nucleosides are 2′-MOE nucleosides. In a further preferred embodiment, said second oligonucleotide comprises, preferably consists of, uniformly modified nucleosides, wherein said modified nucleosides are 2′-OMe nucleosides. In a further preferred embodiment, said second oligonucleotide comprises at least 6 contiguous linked 2′-deoxy nucleosides. In a further preferred embodiment, said second oligonucleotide comprises at least 7 contiguous linked 2′-deoxy nucleosides. In a further preferred embodiment, said second oligonucleotide comprises at least 8 contiguous linked 2′-deoxy nucleosides. In a further preferred embodiment, said second oligonucleotide comprises at least 9 contiguous linked 2′-deoxy nucleosides. In a further preferred embodiment, said second oligonucleotide comprises at least 10 contiguous linked 2′-deoxy nucleosides.

### Nucleobases

In certain embodiments, the first oligonucleotide comprises one or more nucleoside comprising an unmodified nucleobase. In certain embodiments, the second oligonucleotide comprises one or more nucleoside comprising an unmodified nucleobase.

In certain embodiments, the first oligonucleotide or the second oligonucleotide comprises one or more nucleoside comprising a modified nucleobase. In certain embodiments, the first oligonucleotide or the second oligonucleotide comprises one or more nucleoside that does not comprise a nucleobase, referred to as an abasic nucleoside. In certain embodiments, modified nucleobases are selected from 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from 5-methylcytosine, 5-bromouracil, inosine, 5-substituted pyrimidines, 6-azapyrimidines and N-2, N-6 and O-6 substituted purines, including 2-aminopropyladenine, 5-propynyluracil and 5-propynylcytosine. Base analogs also include, but are not limited to, 5-hydroxymethylcytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine, 2-thiocytosine, 5-halouracil and cytosine, 5-propinyluracil and 5-propinylcytosine (and other alkynyl derivatives of pyrimidine bases), 6-azouracil, 6-azocytosine, 6-azothymine, 5-uracil (pseudouracil), 4-thiouracil, 8-halo, 8-amino, 8-thiol, 8-thioalkyl, 8-hydroxyl and other 8-substituted adenines and guanines, 5-halo and particularly 5-bromo, 5-trifluoromethyl and other 5-substituted uracils and cytosine's, 7-methylguanine and 7-methyladenine, 2-F-adenine, 2-amino-adenine, 8-azaguanine and 8-azaadenine, 7-deazaguanine and 7-deaza-adenine, 3-deazaguanine and 3-deaza-adenine, universal bases, tricyclic pyrimidines such as phenoxazine cytidine (1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), and pyridoindole cytidine (2H-pyrido[3′,2′:4,5]pyrrolo[2,3-d]pyrimidin-2-one). Base analogs may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et ah, U.S. Pat. No. 3,687,808, those disclosed in The Concise Encyclopedia Of Polymer Science And Engineering, Kroschwitz, J. I., Ed., John Wiley & Sons, 1990, 858-859; Englisch et al, Angewandte Chemie, International Edition, 1991, 30:613; Sanghvi, Y S, Chapter 15, Antisense Research and Applications, Crooke, S T and Lebleu, B, Eds., CRC Press, 1993, 273-288; and those disclosed in Chapters 6 and 15, Antisense Drug Technology, Crooke S T, Ed., CRC Press, 2008, 163-166 and 442-443. The preparation of modified nucleobases is known in the art and is described in U.S. Pat. Nos. 3,687,808, 4,845,205, 5,130,302, 5,134,066, 5,175,273, 5,367,066, 5,432,272, 5,457,187, 5,459,255, 5,484,908, 5,502,177, 5,525,711, 5,552,540, 5,587,469, 5,594,121, 5,596,091, 5,614,617, 5,645,985, 5,750,692, 5,830,653, 5,763,588, 6,005,096 and 5,681,941.

### Internucleoside Linkages

In certain embodiments, nucleosides of oligonucleotides may be linked together using any internucleoside linkage. In certain embodiments, nucleosides of the first oligonucleotide may be linked together using any internucleoside linkage. In certain embodiments, nucleosides of the second oligonucleotide may be linked together using any internucleoside linkage.

The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, phosphorodiamidates, phosphorothioates (“P═S”), and phosphorodithioates. Representative non-phosphorus containing internucleoside linking groups include but are not limited to methylenemethylimino, thiodiester, thionocarbamate, siloxane and N,N′-dimethylhydrazine. Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Representative chiral internucleoside linkages include but are not limited to alkylphosphonates and phosphorothioates. Methods of preparation of phosphorous-containing and non-phosphorous-containing internucleoside linkages are well known to those skilled in the art.

In an embodiment, the internucleoside linkage of the oligonucleotides of the invention comprise phosphate moieties independently selected from the group consisting of a phosphorothioate, a phosphorodithioate, a phosphorodiester, a phosphotriester, an aminoalkylphosphotriester, a methyl phosphonate, an alkyl phosphonate, a 5′-alkylene phosphonate, a phosphonate, a phosphinate, a phosphoramidate, a phosphorodiamidate, an 3′-aminophosphoramidate, an aminoalkyl phosphoramidate, a thionophosphoramidate, a thionoalkylphosphonate, a thionoalkylphosphotriester, a selenophosphate, and a boranophosphate.

In certain embodiments, essentially each internucleoside linkage is a phosphate internucleoside linkage (P═O). In certain embodiments, essentially each internucleoside linkage of the first oligonucleotide is a phosphate internucleoside linkage (P═O). In certain embodiments, essentially each internucleoside linkage of the second oligonucleotide is a phosphate internucleoside linkage (P═O). In certain embodiments, each internucleoside linkage of the first oligonucleotide is independently selected from a phosphorothioate and phosphate internucleoside linkage. In certain embodiments, each internucleoside linkage of the second oligonucleotide is independently selected from a phosphorothioate, phosphorodiamidate and phosphate internucleoside linkage.

In certain embodiments, the sugar motif of the second oligonucleotide is a gapmer and the internucleoside linkages within the gap are all phosphate internucleoside linkages. In certain such embodiments, some or all of the internucleoside linkages in the wings are unmodified phosphate linkages. In certain embodiments, the terminal internucleoside linkages are modified preferably phosphorothioate linkages. In certain embodiments, the sugar motif of the second oligonucleotide is gapmer-like and the internucleoside linkages within the gap are all phosphate internucleoside linkages. In certain such embodiments, some or all of the internucleoside linkages in the wings are unmodified phosphate linkages or modified phosphorothioate linkages. In certain embodiments, the terminal internucleoside linkages are modified, preferably phosphorothioate linkages.

General methods of preparation of internucleoside linkages for use with oligonucleotides and oligomeric compounds are known in the art, including the methods described in U.S. Pat. Nos. 3,687,808, 4,469,863, 4,476,301, 5,023,243, 5,177,196, 5,188,897, 5,264,423, 5,276,019, 5,278,302, 5,286,717, 5,321,131, 5,399,676, 5,405,939, 5,453,496, 5,455,233, 5,466,677, 5,476,925, 5,519,126, 5,536,821, 5,541,306, 5,550,111, 5,563,253, 5,571,799, 5,587,361, 5,194,599, 5,565,555, 5,527,899, 5,721,218, 5,672,697 and 5,625,050 as well as in Krotz et al, Org. Proc. R&D 2004, 8:852-858.

In a preferred embodiment, only four of said internucleoside linkages comprised by said second oligonucleotide are modified internucleoside linkages, wherein preferably said modified internucleoside linkages are phosphorothioate internucleoside linkages. In a preferred embodiment, only three of said internucleoside linkages comprised by said second oligonucleotide are modified internucleoside linkages, wherein preferably said modified internucleoside linkages are phosphorothioate internucleoside linkages. In a preferred embodiment, only two of said internucleoside linkages comprised by said second oligonucleotide are modified internucleoside linkages, wherein preferably said modified internucleoside linkages are phosphorothioate internucleoside linkages. In a preferred embodiment, only one of said internucleoside linkages comprised by said second oligonucleotide is a modified internucleoside linkage, wherein preferably said modified internucleoside linkage is a phosphorothioate internucleoside linkage. In a further preferred embodiment, all of said internucleoside linkages comprised by said second oligonucleotide are unmodified phosphodiester internucleoside linkages. In a further preferred embodiment, all of said modified internucleoside linkages are at the 5′-end/wing and/or at the 3′-end/wing of said second oligonucleotide. In a further preferred embodiment, all of said modified internucleoside linkages are at the 3′-end/wing of said second oligonucleotide.

### Oligonucleotide Lengths

In certain embodiments, the first and/or second oligonucleotides can have any of a variety of ranges of lengths. In certain embodiments, the first oligonucleotide can have any of a variety of ranges of lengths. In certain embodiments, the second oligonucleotide can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50. For example, in certain embodiments, oligonucleotides consist of 6 to 7, 6 to 8, 6 to 9, 6 to 10, 6 to 11, 6 to 12, 7 to 8, 7 to 9, 7 to 10, 7 to 11, 7 to 12, 8 to 9, 8 to 10, 8 to 11, 8 to 12, 9 to 10, 9 to 11, 9 to 12, 10 to 11, 10 to 12, 11 to 12, 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

### Nucleobase Sequence

In certain embodiments, the first and the second oligonucleotides (unmodified or modified oligonucleotides) are further described by their nucleobase sequence. In certain embodiments the first oligonucleotide has a nucleobase sequence that is complementary to the second oligonucleotide and/or an identified reference nucleic acid such as a target nucleic acid. In certain such embodiments, a region of the first oligonucleotide has a nucleobase sequence that is complementary to a region of the second oligonucleotide or an identified reference nucleic acid such as a target nucleic acid. In certain embodiments, the nucleobase sequence of a region or entire length of the first oligonucleotide is at least 50%, at least 60%, at least 70%, at least 80%, at least 90%, at least 95%, or 100% complementary to the second oligonucleotide or the target nucleic acid. In certain embodiments, the nucleobase sequence of the entire length of the first oligonucleotide is at least 50%, at least 60%, at least 70%, at least 80%, at least 90%, at least 95%, or 100% complementary to the second oligonucleotide or the target nucleic acid.

### Oligomeric Compounds

In certain embodiments, the invention provides oligomeric compounds, which consist of modified or unmodified oligonucleotides, and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups that are attached to either or both ends of an oligonucleotide are terminal groups. In certain such embodiments, conjugate groups or terminal groups are attached at the 3′- and/or 5′-end of oligonucleotides. In certain such embodiments, conjugate groups (or terminal groups) are attached at the 3′-end of oligonucleotides. In certain embodiments, conjugate groups (or terminal groups) are attached at the 5′-end of oligonucleotides. Examples of terminal groups include but are not limited to conjugate groups, capping groups, phosphate moieties and protecting groups.

### Conjugate Groups

In certain embodiments, the first oligonucleotide or the second oligonucleotide are covalently attached to one or more conjugate groups. In certain embodiments, the first oligonucleotide is covalently attached to one or more conjugate groups. In certain embodiments, the second oligonucleotide is covalently attached to one or more conjugate groups. In certain embodiments, the second oligonucleotide is covalently attached to one or more conjugate groups and the first oligonucleotide is not attached to a conjugate group. In certain embodiments, the first oligonucleotide is covalently attached to one or more conjugate groups and the second oligonucleotide is not attached to a conjugate group. In certain embodiments, the first oligonucleotide is not attached to a conjugate group. In certain embodiments, the first oligonucleotide does not comprise a conjugate group. In certain embodiments, the second oligonucleotide is not attached to a conjugate group. In certain embodiments, the second oligonucleotide does not comprise a conjugate group.

In certain embodiments, conjugate groups modify one or more properties of the attached oligonucleotide, including but not limited to pharmacodynamics, pharmacokinetics, stability, binding, absorption, tissue distribution, tissue targeting, cellular distribution, cell internalization, endosomal escape, target binding specificity, resistance to nucleases, cellular uptake, charge and clearance. In certain embodiments, conjugate groups impart a new property on the attached oligonucleotide, e.g., fluorophores or reporter groups that enable detection of the oligonucleotide. In certain embodiments, conjugate groups and conjugate moieties are selected from a lipid group, intercalators, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, lipophilic moieties, coumarins, peptides, antibodies, nanobodies, and oligosaccharides, for example N-acetylgalactosamine. Thus, conjugate groups and conjugate moieties have been described, for example: cholesterol moiety (Letsinger et al, Proc. Natl. Acad. Sci. USA, 1989, 86:6553-6556), cholic acid (Manoharan et al, Bioorg. Med. Chem. Lett., 1994, 4:1053-1060), a thioether (Manoharan et al, Ann. NY. Acad. Sci., 1992, 660:306-309; Manoharan et al., Bioorg. Med. Chem. Lett., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993:75:49-54), a phospholipid (Manoharan et ak, Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid or a palmityl moiety (Mishra et al., Biochim. Biophys. Acta, 1995:1264, 229-237), an octadecylamine or hexylamino-carbonyl-oxycholesterol moiety (Crooke et al., J. Pharmacol. Exp. Ther., 1996, 277:923-937), a tocopherol group (Nishina et al., Molecular Therapy Nucleic Acids, 2015, 4, e220; and Nishina et al., Molecular Therapy, 2008, 16:734-740), or a GalNAc cluster (e.g., WO2014/179620), cell penetrating peptides (Taylor R E and Zahid M, Pharmaceutics, 2020, 12, 225. Gait M J et al, Nucleic Acid Therapeutics 2019, 29:1-12), antibodies (Cuellar T L et al, Nucleic Acids Research, 2015, 43(2):1189-1203), aptamers, squalene or nucleolipids (Benizri S et al, Bioconjug Chem. 2019, 30(2):366-383 and references cited therein).

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes. In certain embodiments, a conjugate moiety comprises an active drug substance, for example, aspirin, warfarin, phenylbutazone, ibuprofen, suprofen, fenbufen, ketoprofen, (S′)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indomethicin, a barbiturate, a cephalosporin, a sulfa drug, an antidiabetic, an antibacterial or an antibiotic.

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group. In certain embodiments, conjugate linkers, including the conjugate linkers described above, are bifunctional linking moieties, e.g., those known in the art to be useful for attaching conjugate groups to parent compounds, such as the first and/or second oligonucleotides described herein. In general, a bifunctional linking moiety comprises at least two functional groups. One of the functional groups is selected to form a bond with to a particular site on a parent compound and the other is selected to form a bond with to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl. Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₂-C₁₀alkyl, substituted or unsubstituted C₂-C₁₀alkenyl or substituted or unsubstituted C₂-C₁₀alkynyl, wherein a non-limiting list of preferred substituent groups includes hydroxyl, amino, alkoxy, carboxy, benzyl, phenyl, nitro, thiol, thioalkoxy, halogen, alkyl, aryl, alkenyl and alkynyl. In certain embodiments, conjugate linkers comprise 2-5 linker-nucleosides, preferably exactly 3 linker-nucleosides.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases. In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In a preferred embodiment, said conjugate moiety is a lipid group, wherein said conjugate linker is a hydrocarbon or a PEG linker, wherein preferably said hydrocarbon or PEG linker is selected from the group consisting of amino-alkyl-phosphorothioate linkers, alpha-carboxylate-amino-alkyl-phosphorothiate linkers, amino-PEG-phosphorothioate linkers and alpha-carboxylate-amino-PEG-phosphorothioate linkers. A conjugate linker according to the invention typically and preferably does not decrease or prevent the binding of the first oligonucleotide to its nucleic acid target. A conjugate linker can include a cleavable group or at least one cleavable bond.

In certain embodiments, said conjugate moiety is a lipid group, wherein the lipid group is a fatty acid derived group. In certain embodiments, the fatty acid derived group comprises a carboxy group. Fatty acids include any saturated or unsaturated fatty acid having a hydrocarbon chain of 4 to 28 carbon atoms, and can contain one or two carboxylic acid groups. A fatty acid that contains two carboxylic acid groups is a dicarboxylic acid. One or two fatty acid ligands can be attached to the oligonucleotide, preferably to the first oligonucleotide, via a conjugate linker.

In certain embodiments, the lipid group is a fatty acid derived group, wherein the fatty acid is any one of the fatty acids presented in Tables 1 and 2.

TABLE 1
Saturated Fatty Acids

| | | | |
|---|---|---|---|
| Butyric acid | Butanoic acid | CH₃(CH₂)₂COOH | C4:0 |
| Valeric acid | Pentanoic acid | CH₃(CH₂)₃COOH | C5:0 |
| Caproic acid | Hexanoic acid | CH₃(CH₂)₄COOH | C6:0 |
| Enanthic acid | Heptanoic acid | CH₃(CH₂)₅COOH | C7:0 |
| Caprylic acid | Octanoic acid | CH₃(CH₂)₆COOH | C8:0 |
| Pelargonic acid | Nonanoic acid | CH₃(CH₂)₇COOH | C9:0 |
| Capric acid | Decanoic acid | CH₃(CH₂)₈COOH | C10:0 |
| Undecylic acid | Undecanoic acid | CH₃(CH₂)₉COOH | C11:0 |
| Lauric acid | Dodecanoic acid | CH₃(CH₂)₁₀COOH | C12:0 |
| Tridecylic acid | Tridecanoic acid | CH₃(CH₂)₁₁COOH | C13:0 |
| Myristic acid | Tetradecanoic acid | CH₃(CH₂)₁₂COOH | C14:0 |
| Pentadecylic acid | Pentadecanoic acid | CH₃(CH₂)₁₃COOH | C15:0 |
| Palmitic acid | Hexadecanoic acid | CH₃(CH₂)₁₄COOH | C16:0 |
| Margaric acid | Heptadecanoic acid | CH₃(CH₂)₁₅COOH | C17:0 |
| Stearic acid | Octadecanoic acid | CH₃(CH₂)₁₆COOH | C18:0 |
| Nonadecylic acid | Nonadecanoic acid | CH₃(CH₂)₁₇COOH | C19:0 |
| Arachidic acid | Eicosanoic acid | CH₃(CH₂)₁₈COOH | C20:0 |
| Heneicosylic acid | Heneicosanoic acid | CH₃(CH₂)₁₉COOH | C21:0 |
| Behenic acid | Docosanoic acid | CH₃(CH₂)₂₀COOH | C22:0 |
| Tricosylic acid | Tricosanoic acid | CH₃(CH₂)₂₁COOH | C23:0 |
| Lignoceric acid | Tetracosanoic acid | CH₃(CH₂)₂₂COOH | C24:0 |
| Pentacosylic acid | Pentacosanoic acid | CH₃(CH₂)₂₃COOH | C25:0 |
| Cerotic acid | Hexacosanoic acid | CH₃(CH₂)₂₄COOH | C26:0 |
| Heptacosylic acid | Heptacosanoic acid | CH₃(CH₂)₂₅COOH | C27:0 |
| Montanic acid | Octacosanoic acid | CH₃(CH₂)₂₆COOH | C28:0 |

TABLE 2
Unsaturated fatty acids

| | | | | |
|---|---|---|---|---|
| α-Linolenic acid | C18:3 | Δ^{9, 12, 15} | CH₃CH₂CH═CHCH₂CH═CHCH₂CH═CH(CH₂)₇COOH | cis |
| Stearidonic acid | C18:4 | Δ^{6, 9, 12, 15} | CH₃CH₂CH═CHCH₂CH═CHCH₂CH═CHCH₂CH═CH(CH₂)₄COOH | cis |
| Eicosapentaenoic acid | C20:5 | Δ^{5, 8, 11, 14, 17} | CH₃CH₂CH═CHCH₂CH═CHCH₂CH═CHCH₂CH═CHCH₂CH═CH(CH₂)₃COOH | cis |
| Docosahexaenoic acid | C22:6 | Δ^{4, 7, 10, 13, 16, 19} | CH₃CH₂CH═CHCH₂CH═CHCH₂CH═CHCH₂CH═CHCH₂CH═CHCH₂CH═CH(CH₂)₂COOH | cis |
| Linoleic acid | C18:2 | Δ^{9, 12} | CH₃(CH₂)₄CH═CHCH₂CH═CH(CH₂)₇COOH | cis |
| Linolelaidic acid | C18:2 | | CH₃(CH₂)₄CH═CHCH₂CH═CH(CH₂)₇COOH | trans |
| γ-Linolenic acid | C18:3 | Δ^{6, 9, 12} | CH₃(CH₂)₄CH═CHCH₂CH═CHCH₂CH═CH(CH₂)₄COOH | cis |
| Dihomo-γ-linolenic acid | C20:3 | Δ^{8, 11, 14} | CH₃(CH₂)₄CH═CHCH₂CH═CHCH₂CH═CH(CH₂)₆COOH | cis |
| Arachidonic acid | C20:4 | Δ^{5, 8, 11, 14} | CH₃(CH₂)₄CH═CHCH₂CH═CHCH₂CH═CHCH₂CH═CH(CH₂)₃COOH | cis |
| Docosatetraenoic acid | C22:4 | Δ^{7, 10, 13, 16} | CH₃(CH₂)₄CH═CHCH₂CH═CHCH₂CH═CHCH₂CH═CH(CH₂)₅COOH | cis |
| Palmitoleic acid | C16:1 | Δ⁹ | CH₃(CH₂)₅CH═CH(CH₂)₇COOH | cis |
| Vaccenic acid | C18:1 | Δ¹¹ | CH₃(CH₂)₅CH═CH(CH₂)₉COOH | trans |
| Paullinic acid | C20:1 | Δ¹³ | CH₃(CH₂)₅CH═CH(CH₂)₁₁COOH | cis |
| Oleic acid | C18:1 | Δ⁹ | CH₃(CH₂)₇CH═CH(CH₂)₇COOH | cis |
| Elaidic acid | C18:1 | Δ⁹ | CH₃(CH₂)₇CH═CH(CH₂)₇COOH | trans |
| Gondoic acid | C20:1 | Δ¹¹ | CH₃(CH₂)₇CH═CH(CH₂)₉COOH | cis |
| Erucic acid | C22:1 | Δ¹³ | CH₃(CH₂)₇CH═CH(CH₂)₁₁COOH | cis |
| Nervonic acid | C24:1 | Δ¹⁵ | CH₃(CH₂)₇CH═CH(CH₂)₁₃COOH | cis |
| Mead acid | C20:3 | Δ^{5, 8, 11} | CH₃(CH₂)₇CH═CHCH₂CH═CHCH₂CH═CH(CH₂)₃COOH | cis |

In one embodiment, said lipid group is a saturated fatty acid derived group having a hydrocarbon chain of 8 to 24 carbon atoms. In certain embodiments, said lipid group is a saturated fatty acid derived group, wherein said fatty acid is selected from the group consisting of octanoic acid, decanoic acid, dodecanoic acid, tetradecanoic acid, hexadecanoic acid, octadecanoic acid, eicosanoic acid, docosanoic acid and tetracosanoic acid. In one embodiment, said lipid group is a saturated fatty acid derived group, wherein said fatty acid is hexadecanoic acid. In one embodiment, said fatty acid derived group is attached to the oligonucleotide, preferably to the first oligonucleotide, via the conjugate linker, preferably on the 3′ end of the oligonucleotide. In one embodiment, said fatty acid derived group is attached to the oligonucleotide, preferably to the first oligonucleotide, via the conjugate linker, preferably on the 3′ end oligonucleotide.

In one embodiment, said lipid group is an unsaturated fatty acid derived group having a hydrocarbon chain of 8 to 24 carbon atoms. In certain embodiments, said lipid group is an unsaturated fatty acid derived group, wherein said fatty acid is selected from the group consisting of myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid and docosahexaenoic acid.

In some embodiments, the conjugate linker is connected to the lipid group via an amide bond. For hydrocarbon conjugate linkers, the conjugate linker comprises 2-20 carbons, for example, 2, 3, 4, 5, 6, 7, 8 9 or 10 carbons. For polyethylene glycol (PEG) conjugate linkers, the conjugate linker comprises 1-20 ethylene glycol subunits, for example, 1, 2, 3, 4, 5, 6, 7, 8 9 or 10 ethylene glycol repeats. A conjugate linker can be a hydrocarbon linker or a polyethylene glycol (PEG) linker. A conjugate linker according to the invention, wherein the oligonucleotide, preferably said first oligonucleotide, is attached to a phosphorous moiety of the conjugate linker, and the lipid group, for example a fatty acid derived group, is attached to Y, can have, for example, the general structure shown below:

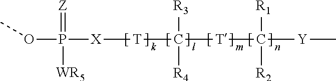

wherein:
If Y=NH then the fatty acid-derived group is connected via an amide bond;
If n=1, R₁ can be, for example, CO₂H and R₂ can be, for example, H;
T′ can be —CH₂—CH₂—O with m being the number of ethylene glycol repeats;
T can be a biocleavable entity such as a disulfide group, and k is equal to 1,
wherein, in certain embodiments,
X can be oxygen or NH;
Z can be O or S; and
WR₅ can be OH or SH.

Conjugate linkers useful according to the invention include but are not limited to the following:
amino-alkyl-phosphorothioate conjugate linker:

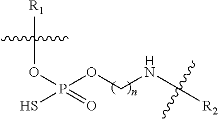

R₁=oligonucleotide
R₂=conjugated lipid group

wherein n is preferably an integer of 2 to 12, preferably of 4 to 10. In one embodiment, n is an integer of 2, 3, 4, 5, 6, 7, 8, 9, 10, 11 or 12. In one embodiment, n is 6;
alpha-carboxylate-amino-alkyl-phosphorothioate conjugate linker:

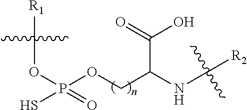

R₁=oligonucleotide
R₂=conjugated lipid group

wherein n is preferably an integer of 2 to 12, preferably of 4 to 10. In one embodiment, n is an integer of 2, 3, 4, 5, 6, 7, 8, 9, 10, 11 or 12. In one embodiment, n is 6;
amino-PEG-phosphorothioate conjugate linker:

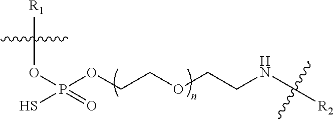

R₁=oligonucleotide
R₂=conjugated lipid group

wherein n is preferably an integer of 1 to 8. In one embodiment, n is an integer of 1, 2, 3, 4, 5, 6, 7 or 8; and
alpha-carboxylate-amino-PEG-phosphorothioate conjugate linker:

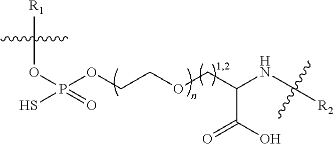

R₁=oligonucleotide
R₂=conjugated lipid group

wherein n is preferably an integer of 1 to 8. In one embodiment, n is an integer of 1, 2, 3, 4, 5, 6, 7 or 8.

Thus, in one embodiment said conjugate linker is selected from the group consisting of
(i) an amino-alkyl-phosphorothioate linker;
(ii) an alpha-carboxylate-amino-alkyl-phosphorothioate linker;
(iii) an amino-PEG-phosphorothioate linker, and
(iv) alpha-carboxylate-amino-PEG-phosphorothioate linker all as defined above in provided formula.

In one embodiment said conjugate linker is an amino-alkyl-phosphorothioate linker, wherein said amino alkyl phosphorothioate linker has the structure presented below.

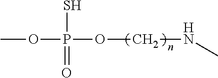

wherein n is an integer of 2 to 12, preferably of 4 to 10. In one embodiment, n is an integer of 2, 3, 4, 5, 6, 7, 8, 9, 10, 11 or 12. In one embodiment, n is 6.

In one embodiment said conjugate linker is an amino-PEG-phosphorothioate linker having the structure provided below.

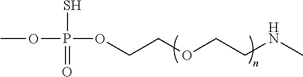

wherein n is preferably an integer of 1 to 8. In one embodiment, n is an integer of 1, 2, 3, 4, 5, 6, 7 or 8.

In one embodiment said conjugate linker is an alpha-carboxylate-amino-alkyl-phosphorothioate linker having the structure provided below.

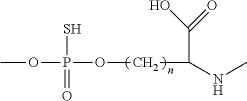

wherein n is preferably an integer of 2 to 12, preferably of 4 to 10. In one embodiment, n is an integer of 2, 3, 4, 5, 6, 7, 8, 9, 10, 11 or 12. In one embodiment, n is 6.

In one embodiment said conjugate linker is an alpha-carboxylate-amino-PEG-phosphorothioate linker having the structure provided below.

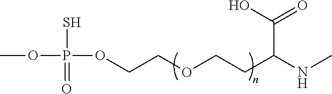

wherein n is preferably an integer of 1 to 8. In one embodiment, n is an integer of 2, 3, 4, 5, 6, 7 or 8.

In one embodiment said conjugate linker is a linker that is conformationally restrained, for example, based on hydroxyproline, for example,

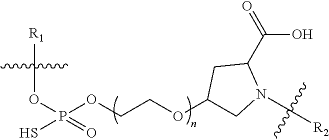

R₁=oligonucleotide
R₂=conjugated lipid group

wherein n is preferably an integer of 1 to 8. In one embodiment, n is an integer of 2, 3, 4, 5, 6, 7 or 8.

The conjugate linker can be attached to the terminal OH group of the oligonucleotide via, for example, a thiophosphate group or phosphate group. In one embodiment, the conjugate linker is attached to the 5′ terminal OH group of the oligonucleotide via, for example, a thiophosphate group. In one embodiment, the linker is attached to the 3′ terminal OH group of the oligonucleotide via, for example, a thiophosphate group.

In one embodiment said conjugate linker is an alpha-carboxylate-amino linker having, for example, the structure:

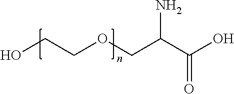

wherein n is preferably an integer of 1 to 8. In one embodiment, n is an integer of 2, 3, 4, 5, 6, 7 or 8.

In one embodiment said conjugate linker is a 2-amino-6-hydroxy-4-oxahexanoic acid linker wherein n=1.

In some embodiments, a fatty acid conjugated solid phase support may be used for the coupling of a fatty acid to the oligonucleotide. An example of a solid phase support which may be used for the coupling of a fatty acid to the oligonucleotide has the structure:

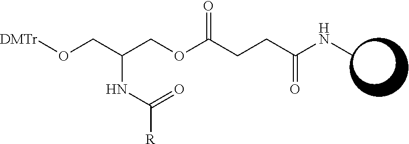

wherein R—CO is a fatty acid moiety and the shaded circle is the solid phase support.

In other embodiments, a solid phase support which may be used for the coupling of a fatty acid to the oligonucleotide has the structure (AM Chemicals, LLC, Oceanside, CA):

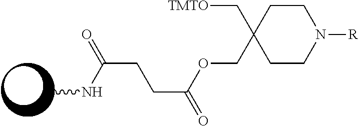

wherein R is a fatty acid moiety and the shaded circle is the solid phase support.

In certain embodiments, the conjugate linker contains a cleavable bond, for example, a disulfide bond, an acid cleavable hydrazone bond, or a protease cleavable moiety.

In certain embodiments, at least one of the first and second oligomeric compounds comprises a conjugate group (as described above). Typically and preferably, the first oligomeric compound comprises a conjugate group. The conjugate group may be attached at either the 3′- or 5′-end of the oligomeric compound. In certain embodiments, a conjugate group is attached to both ends.

### Antisense Activity

In certain embodiments, the present invention provides compounds, which comprise a first oligomeric compound, preferably being an antisense compound, comprising a first antisense oligonucleotide having a nucleobase sequence complementary to that of a target nucleic acid. In certain embodiments, said first oligomeric compound comprises or consists of a first oligonucleotide and optionally a conjugate group. In certain embodiments, the inventive compounds are double-stranded comprising a first oligomeric compound which comprises a first oligonucleotide having a region complementary to a target nucleic acid and a second oligomeric compound which comprises a second oligonucleotide having a region complementary to the first oligonucleotide. The first oligomeric compound of such double stranded antisense compounds typically comprises or consists of a first modified oligonucleotide and optionally a conjugate group. The second oligonucleotide of the second oligomeric compound of such double-stranded inventive compound may be modified or unmodified and optionally comprises a conjugate group. Either or both oligomeric compounds of the inventive double-stranded antisense compound may comprise a conjugate group. The oligomeric compounds of double-stranded antisense compounds may include non-complementary, typically and preferably, overhanging nucleosides. In certain embodiments, oligomeric compounds of antisense compounds are capable of hybridizing to a target nucleic acid, resulting in at least one antisense activity. In certain embodiments, the inventive antisense compounds selectively affect one or more target nucleic acid, typically and preferably one target nucleic acid. Such selective antisense compounds comprise a nucleobase sequence that hybridizes to one or more target nucleic acid, typically and preferably to one target nucleic acid, resulting in one or more, typically and preferably one, desired antisense activity.

In certain embodiments, the first oligonucleotide is capable of modulating splicing of a given target nucleic acid. In certain embodiments, the first oligomeric compound comprises a conjugate group, and thus the first oligomeric compound improves a property of the inventive compound compared to the property in the absence of the conjugate group comprised by said first oligomeric compound. In certain embodiments, the improved property is one or more of distribution to a target tissue, uptake into a target cell, potency, and/or efficacy.

In certain embodiments, the target tissue is muscle tissue. In certain embodiments, the target tissue is other than liver (extra-hepatic).

### Target Nucleic Acids

In certain embodiments, the target nucleic acid is an endogenous RNA molecule. In certain embodiments, the target nucleic acid encodes a protein. In certain such embodiments, the target nucleic acid is selected from: an mRNA and a pre-mRNA, including intronic, exonic and untranslated regions. In certain embodiments, the target RNA is an mRNA. In certain embodiments, the target nucleic acid is a pre-mRNA. In certain such embodiments, the target region is entirely within an intron. In certain embodiments, the target region spans an intron/exon junction. In certain embodiments, the target region is at least 50% within an intron.

In certain embodiments, the target nucleic acid is a non-coding RNA. In certain such embodiments, the target non-coding RNA is selected from: a long-non-coding RNA, a short non-coding RNA, an intronic RNA molecule, a snoRNA, a scaRNA, a microRNA (including pre-microRNA and mature microRNA), a ribosomal RNA, and promoter directed RNA. In certain embodiments, the target nucleic acid is a nucleic acid other than a mature mRNA. In certain embodiments, the target nucleic acid is a nucleic acid other than a mature mRNA or a microRNA. In certain embodiments, the target nucleic acid is a non-coding RNA other than a microRNA. In certain embodiments, the target nucleic acid is a non-coding RNA other than a microRNA or an intronic region of a pre-mRNA. In certain embodiments, the target nucleic acid is a long non-coding RNA. In certain embodiments, the target nucleic acid is a non-coding RNA associated with splicing of other pre-mRNAs. In certain embodiments, the target nucleic acid is a nuclear-retained non-coding RNA.

In certain embodiments, the first oligonucleotide is complementary to the target nucleic acid over the entire length of the oligonucleotide. In certain embodiments, the first oligonucleotide is 99% complementary to the target nucleic acid. In certain embodiments the first oligonucleotide is 95% complementary to the target nucleic acid. In certain embodiments, the first oligonucleotide is 90% complementary to the target nucleic acid. In certain embodiments, the first oligonucleotide is 85% complementary to the target nucleic acid. In certain embodiments, the first oligonucleotide is 80% complementary to the target nucleic acid.

In certain embodiments, the first oligonucleotide is at least 80% complementary to the target nucleic acid over the entire length of the oligonucleotide and comprise a region that is 100% or fully complementary to a target nucleic acid. In certain such embodiments, the region of full complementarity is from 6 to 20 nucleobases in length. In certain such embodiments, the region of full complementarity is from 10 to 18 nucleobases in length. In certain such embodiments, the region of full complementarity is from 18 to 20 nucleobases in length.

In certain embodiments, the first oligonucleotide comprises one or more mismatched nucleobases relative to the target nucleic acid. In certain such embodiments, antisense activity against the target is reduced by such mismatch, but activity against a non-target is reduced by a greater amount. Thus, in certain such embodiments selectivity of the inventive compounds and thus the antisense compound is improved.

In certain embodiments, the inventive compounds comprise or consist of a first oligonucleotide comprising a region that is complementary to a target nucleic acid, wherein the target nucleic acid is expressed in an extra-hepatic tissue. Extra-hepatic tissues include, but are not limited to: skeletal muscle, cardiac muscle, smooth muscle, adipose, white adipose, spleen, bone, intestine, adrenal, testes, ovary, pancreas, pituitary, prostate, skin, uterus, bladder, brain, glomerulus, distal tubular epithelium, breast, lung, heart, kidney, ganglion, frontal cortex, spinal cord, trigeminal ganglia, sciatic nerve, dorsal root ganglion, epididymal fat, diaphragm, pancreas, and colon. Extra-hepatic tissues include, but are not limited to CNS tissues, for example, the brain.

### Pharmaceutical Compositions

In certain embodiments, the present invention provides pharmaceutical compositions comprising the inventive compound or a salt thereof. In certain such embodiments, the pharmaceutical composition comprises a suitable pharmaceutically acceptable diluent or carrier. In certain embodiments, a pharmaceutical composition comprises a sterile saline solution, and the inventive compound. In certain embodiments, such pharmaceutical composition consists of a sterile saline solution and the inventive compound. In certain embodiments, a pharmaceutical composition comprises the inventive compound and sterile water. In certain embodiments, a pharmaceutical composition consists of the inventive and sterile water. In certain embodiments, a pharmaceutical composition comprises the inventive compound and phosphate-buffered saline (PBS). In certain embodiments, a pharmaceutical composition consists of the inventive compound and sterile PBS. In certain embodiments, pharmaceutical compositions comprise the inventive compound and one or more excipients. In certain such embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

Lipid moieties have been used in nucleic acid therapies in a variety of methods. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

Each of the literature and patent publications listed herein is incorporated by reference in its entirety. Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as a or b such as for sugar anomers, or as (D) or (L), such as for amino acids, etc. Included in the compounds provided herein are all such possible isomers, including their racemic and optically pure forms, unless specified otherwise. Likewise, all cis- and trans isomers and tautomeric forms are also included unless otherwise indicated. Unless otherwise indicated, compounds described herein are intended to include corresponding salt forms.

### Exon Skipping Efficacy

The present invention provides for inventive duplexes that can efficiently promote exon skipping in target nucleic acids. As set forth herein, in some embodiment the first oligonucleotide of the inventive duplexes is a gapmer. As set forth in Example 8, gapmers comprising abcDNA nucleosides in the wings, in particular gapmers comprising 4 or 5 abcDNA nucleosides in the wings, showed increased affinity for complementary RNA when compared with natural DNA.

As set forth in Example 9, without wishing to be bound by theory, the data suggest that abcDNA gapmers induce RNA cleavage with comparable activity as natural DNA or as conventional MOE gapmers.

As set forth in Example 10, without wishing to be bound by theory, the data suggest that abcDNA gapmers comprising PS-DNA in the gap (abcDNA-gap5, SEQ ID NO: 67), exhibit high biostability. abcDNA gapmers comprising PO-DNA in the gap (abcDNA-gap4, SEQ ID NO: 66 or abcDNA-gap6, SEQ ID NO: 68), exhibit moderate biostability, better than DNA (DNA-gap2, SEQ ID NO: 74), and slightly better than conventional MOE gapmers (MOE-gap, SEQ ID NO:72). The biostability of abcDNA gapmers comprising PO-DNA in the gap (abcDNA-gap4, SEQ ID NO:66), is substantially increased in duplex with complementary RNA (RNA6, SEQ ID NO:80). The biostability was further improved by using a complementary RNA containing 2 abcDNA nucleotides at each terminal position (abcDNA-pass1, SEQ ID NO: 70). Accordingly, in some preferred embodiments, inventive duplexes of the present disclosure comprise a first oligonucleotide wherein the first oligonucleotide is a gapmer, and a second oligonucleotide wherein the second oligonucleotide is a complementary RNA oligonucleotide.

### PREFERRED EMBODIMENTS

The present disclosure provides the following non-limiting preferred embodiments: Embodiment 1: A compound comprising a first oligomeric compound and a second oligomeric compound, wherein the first oligomeric compound comprises a first oligonucleotide and said second oligomeric compound comprises a second oligonucleotide, wherein
said first oligonucleotide comprises at least one abc-DNA nucleoside, and wherein said first oligonucleotide has a nucleobase sequence that is complementary to a nucleic acid target, and wherein preferably said first oligonucleotide is an antisense oligonucleotide; and wherein
said second oligonucleotide has a nucleobase sequence that is complementary to the nucleobase sequence of the first oligonucleotide.

Embodiment 2: A compound comprising a first oligomeric compound and a second oligomeric compound, wherein the first oligomeric compound comprises a first oligonucleotide and said second oligomeric compound comprises a second oligonucleotide, and wherein
said first oligonucleotide has a nucleobase sequence that is complementary to a nucleic acid target, and wherein preferably said first oligonucleotide is an antisense oligonucleotide; and wherein said second oligonucleotide has a nucleobase sequence that is complementary to the nucleobase sequence of the first oligonucleotide; and wherein
the affinity of said first oligonucleotide to said second oligonucleotide is lower than the affinity of said first oligonucleotide to the fully complementary unmodified RNA oligonucleotide of said first oligonucleotide, wherein preferably said lower affinity corresponds to a lower melting temperature Tm of the duplex of said first and second oligonucleotide as compared to the melting temperature Tm of the duplex of said first oligonucleotide and the fully complementary unmodified RNA oligonucleotide of said first oligonucleotide, and wherein further preferably said melting temperatures Tm are determined as described in Example 2;
or wherein
the biostability of said second oligonucleotide is lower than the biostability of said first oligonucleotide, wherein preferably said lower biostability corresponds to a lower half-life stability in mouse serum, wherein further preferably said lower biostability corresponds to a lower half-life stability in mouse serum as determined by AEX-chromatography after denaturating the duplex of said first and said second oligonucleotide, and again further preferably said lower biostability corresponds to a lower half-life stability in mouse serum as determined by AEX-chromatography after denaturating the duplex of said first and said second oligonucleotide as described in Example 3.

Embodiment 3: A compound comprising a first oligomeric compound and a second oligomeric compound, wherein the first oligomeric compound comprises a first oligonucleotide and said second oligomeric compound comprises a second oligonucleotide, wherein
said first oligonucleotide is a gapmer comprising at least one abc-DNA nucleoside, and wherein said first oligonucleotide has a nucleobase sequence that is complementary to a nucleic acid target, and wherein preferably said first oligonucleotide is an antisense oligonucleotide; and wherein
said second oligonucleotide has a nucleobase sequence that is complementary to the nucleobase sequence of the first oligonucleotide.

Embodiment 4: A compound comprising a first oligomeric compound and a second oligomeric compound, wherein the first oligomeric compound comprises a first oligonucleotide and said second oligomeric compound comprises a second oligonucleotide, wherein
said first oligonucleotide has a nucleobase sequence that is complementary to a nucleic acid target, and wherein preferably said first oligonucleotide is an antisense oligonucleotide; and wherein said second oligonucleotide has a nucleobase sequence that is complementary to the nucleobase sequence of the first oligonucleotide; and wherein
the affinity of said first oligonucleotide to said second oligonucleotide is lower than the affinity of said first oligonucleotide to the fully complementary unmodified RNA oligonucleotide of said first oligonucleotide, wherein preferably said lower affinity corresponds to a lower melting temperature Tm of the duplex of said first and second oligonucleotide as compared to the melting temperature Tm of the duplex of said first oligonucleotide and the fully complementary unmodified RNA oligonucleotide of said first oligonucleotide, and wherein further preferably said melting temperatures Tm are determined as described in Example 2.

Embodiment 5: A compound comprising a first oligomeric compound and a second oligomeric compound, wherein the first oligomeric compound comprises a first oligonucleotide and said second oligomeric compound comprises a second oligonucleotide, wherein
said first oligonucleotide has a nucleobase sequence that is complementary to a nucleic acid target, and wherein preferably said first oligonucleotide is an antisense oligonucleotide; and wherein said second oligonucleotide has a nucleobase sequence that is complementary to the nucleobase sequence of the first oligonucleotide; and wherein
the biostability of said second oligonucleotide is lower than the biostability of said first oligonucleotide, wherein preferably said lower biostability corresponds to a lower half-life stability in mouse serum, wherein further preferably said lower biostability corresponds to a lower half-life stability in mouse serum as determined by AEX-chromatography after denaturating the duplex of said first and said second oligonucleotide, and again further preferably said lower biostability corresponds to a lower half-life stability in mouse serum as determined by AEX-chromatography after denaturating the duplex of said first and said second oligonucleotide as described in Example 3.

Embodiment 6: A compound comprising a first oligomeric compound and a second oligomeric compound, wherein the first oligomeric compound comprises a first oligonucleotide and said second oligomeric compound comprises a second oligonucleotide, wherein
said first oligonucleotide has a nucleobase sequence that is complementary to a nucleic acid target, and wherein preferably said first oligonucleotide is an antisense oligonucleotide; and wherein said second oligonucleotide has a nucleobase sequence that is complementary to the nucleobase sequence of the first oligonucleotide.

Embodiment 7: The compound of any one of the preceding embodiments, wherein said first oligonucleotide is an antisense oligonucleotide.

Embodiment 7: The compound of any one of the preceding embodiments, wherein said first oligonucleotide is a modified oligonucleotide.

Embodiment 9: The compound of any one of the preceding embodiments, wherein said first oligonucleotide is able to modify the activity of said nucleic acid target.

Embodiment 10: The compound of any one of the preceding embodiments, wherein said first oligonucleotide has an antisense effect on said nucleic acid target.

Embodiment 11: The compound of any one of the preceding embodiments, wherein said first oligonucleotide comprises at least one modified nucleoside, wherein said modified nucleoside comprises a modified sugar moiety selected from the group consisting of (i) a non-bicyclic modified sugar moiety comprising a furanosyl ring with one or more acyclic substituent at the 2′, 4′, and/or 5′ position, (ii) a bicyclic sugar moiety, (iii) a tricyclic sugar moiety and (iv) a sugar surrogate.

Embodiment 12: The compound of any one of the preceding embodiments, wherein said first oligonucleotide comprises at least one modified nucleoside, wherein said modified nucleoside comprises a non-bicyclic modified sugar moiety comprising a furanosyl ring with an acyclic substituent at the 2′ position. In a preferred embodiment, said acyclic substituent at the 2′ position is selected from 2′-F, 2′-OH, 2′-propargyl, 2′-O-propylamino, 2′-NH₂, 2′-OCH₃ (“OMe” or “O-methyl”), and 2′-O(CH₂)₂OCH₃ (“MOE”).

Embodiment 13: The compound of any one of the preceding embodiments, wherein said first oligonucleotide comprises at least one modified nucleoside, and wherein said modified nucleoside comprises a non-bicyclic modified sugar moiety comprising a furanosyl ring with an acyclic substituent at the 2′ position, wherein said acyclic substituent at the 2′ position is 2′-O(CH₂)₂OCH₃ (“MOE”).

Embodiment 14: The compound of any one of the preceding embodiments, wherein said first oligonucleotide comprises at least one modified nucleoside, wherein said modified nucleoside comprises a bicyclic sugar moiety, wherein said bicyclic sugar moiety comprises a bridge between the 4′ and the 3′ furanose ring atoms or a bridge between the 4′ and the 2′ furanose ring atoms.

Embodiment 15: The compound of any one of the preceding embodiments, wherein said first oligonucleotide comprises at least one modified nucleoside, wherein said modified nucleoside comprises a tricyclic sugar moiety.

Embodiment 16: The compound of any one of the preceding embodiments, wherein said first oligonucleotide comprises at least one modified nucleoside, wherein said modified nucleoside comprises a sugar surrogate.

Embodiment 17: The compound of any one of the preceding embodiments, wherein said first oligonucleotide comprises at least one modified nucleoside, wherein said modified nucleoside comprises a sugar surrogate, and wherein said sugar surrogate is a morpholino.

Embodiment 18: The compound of any one of the preceding embodiments, wherein each nucleoside of said first oligonucleotide comprises a modified nucleoside, wherein said modified nucleoside comprises a modified sugar moiety selected from the group consisting of (i) a non-bicyclic modified sugar moiety comprising a furanosyl ring with one or more acyclic substituent at the 2′, 4′, and/or 5′ position, (ii) a bicyclic sugar moiety, (iii) a tricyclic sugar moiety and (iv) a sugar surrogate.

Embodiment 19: The compound of any one of the preceding embodiments, wherein each nucleoside of said first oligonucleotide comprises at least one modified nucleoside, wherein said modified nucleoside comprises a non-bicyclic modified sugar moiety comprising a furanosyl ring with an acyclic substituent at the 2′ position. In a preferred embodiment, said acyclic substituent at the 2′ position is selected from 2′-F, 2′-OH, 2′-propargyl, 2′-O-propylamino, 2′-NH₂, 2′-OCH₃ (“OMe” or “O-methyl”), and 2′-O(CH₂)₂OCH₃ (“MOE”).

Embodiment 20: The compound of any one of the preceding embodiments, wherein each nucleoside of said first oligonucleotide comprises a non-bicyclic modified sugar moiety comprising a furanosyl ring with an acyclic substituent at the 2′ position, wherein said acyclic substituent at the 2′ position is 2′-O(CH₂)₂OCH₃ (“MOE”).

Embodiment 21: The compound of any one of the preceding embodiments, wherein each nucleoside of said first oligonucleotide comprises a bicyclic sugar moiety, wherein said bicyclic sugar moiety comprises a bridge between the 4′ and the 3′ furanose ring atoms or a bridge between the 4′ and the 2′ furanose ring atoms.

Embodiment 22: The compound of any one of the preceding embodiments, wherein each nucleoside of said first oligonucleotide comprises a tricyclic sugar moiety.

Embodiment 23: The compound of any one of the preceding embodiments, wherein each nucleoside of said first oligonucleotide comprises a sugar surrogate.

Embodiment 24: The compound of any one of the preceding embodiments, wherein each nucleoside of said first oligonucleotide comprises a sugar surrogate, and wherein said sugar surrogate is a morpholino.

Embodiment 25: The compound of any one of the preceding embodiments, wherein said first oligonucleotide is a modified oligonucleotide, and wherein said modified first oligonucleotide comprises at least one modified nucleoside, wherein said modified nucleoside is selected from the group consisting of a 2′-MOE nucleoside, 2′-OMe nucleoside, an abc-DNA nucleoside, a LNA, a cET, cMOE, a CeNA, a HNA, a ANA, a FANA, a TNA, a tcDNA, a PNA, a GNA, a morpholino or a modified morpholino.

Embodiment 26: The compound of any one of the preceding embodiments, wherein said first oligonucleotide comprises at least one modified nucleoside, wherein said modified nucleoside is selected from the group consisting of a 2′-MOE nucleoside, an abc-DNA nucleoside and a morpholino.

Embodiment 27: The compound of any one of the preceding embodiments, wherein said first oligonucleotide comprises at least one modified nucleoside, wherein said modified nucleoside comprises a 2′-MOE sugar moiety

Embodiment 28: The compound of any one of the preceding embodiments, wherein said first oligonucleotide comprises at least one modified nucleoside, wherein said modified nucleoside is an abc-DNA nucleoside.

Embodiment 29: The compound of any one of the preceding embodiments, wherein said first oligonucleotide comprises at least one modified nucleoside, wherein said modified nucleoside is a morpholino.

Embodiment 30: The compound of any one of the preceding embodiments, wherein each nucleoside of said first oligonucleotide is selected from the group consisting of a 2′-MOE nucleoside, 2′-OMe nucleoside, an abc-DNA nucleoside, a LNA, a cET, cMOE, a CeNA, a HNA, a ANA, a FANA, a TNA, a tcDNA, a PNA, a GNA, a morpholino or a modified morpholino.

Embodiment 31: The compound of any one of the preceding embodiments, wherein each nucleoside of said first oligonucleotide is selected from the group consisting of a 2′-MOE nucleoside, an abc-DNA nucleoside and a morpholino.

Embodiment 32: The compound of any one of the preceding embodiments, wherein each nucleoside of said first oligonucleotide comprises a 2′-MOE sugar moiety.

Embodiment 33: The compound of any one of the preceding embodiments, wherein each nucleoside of said first oligonucleotide is a 2′-MOE nucleoside.

Embodiment 34: The compound of any one of the preceding embodiments, wherein each nucleoside of said first oligonucleotide is an abc-DNA nucleoside.

Embodiment 35: The compound of any one of the preceding embodiments, wherein each nucleoside of said first oligonucleotide is a morpholino.

Embodiment 36: The compound of any one of the preceding embodiments, wherein said first oligonucleotide is a modified oligonucleotide, and wherein said modified first oligonucleotide is a uniformly modified oligonucleotide.

Embodiment 37: The compound of any one of the preceding embodiments, wherein said first oligonucleotide is a modified oligonucleotide, and wherein each nucleobase of said modified first oligonucleotide is an unmodified nucleobase.

Embodiment 38: The compound of any one of the preceding embodiments, wherein each sugar moiety of said second oligonucleotide is an unmodified DNA sugar moiety.

Embodiment 39: The compound of any one of the preceding embodiments, wherein each sugar moiety of said second oligonucleotide is an unmodified DNA sugar moiety, wherein at most five, preferably at most four of all internucleoside linkages of said second oligonucleotide are phosphorothioate internucleoside linkages, wherein preferably said phosphorothioate internucleoside linkages are positioned at the 5′-end and/or at the 3′-end of said second oligonucleotide.

Embodiment 40: The compound of any one of the preceding embodiments, wherein each sugar moiety of said second oligonucleotide is an unmodified DNA sugar moiety, wherein at most five, preferably at most four of all internucleoside linkages of said second oligonucleotide are modified internucleoside linkages, wherein preferably said modified internucleoside linkages are positioned at the 5′-end and/or at the 3′-end of said second oligonucleotide.

Embodiment 41: The compound of any one of the preceding embodiments, wherein each sugar moiety of said second oligonucleotide is an unmodified DNA sugar moiety, wherein at most three of all internucleoside linkages of said second oligonucleotide are phosphorothioate internucleoside linkages, wherein preferably said phosphorothioate internucleoside linkages are positioned at the 5′-end and/or at the 3′-end of said second oligonucleotide.

Embodiment 42: The compound of any one of the preceding embodiments, wherein each sugar moiety of said second oligonucleotide is an unmodified DNA sugar moiety, wherein at most three of all internucleoside linkages of said second oligonucleotide are modified internucleoside linkages, wherein preferably said modified internucleoside linkages are positioned at the 5′-end and/or at the 3′-end of said second oligonucleotide.

Embodiment 43: The compound of any one of the preceding embodiments, wherein each sugar moiety of said second oligonucleotide is an unmodified DNA sugar moiety, wherein at most two of all internucleoside linkages of said second oligonucleotide are phosphorothioate internucleoside linkages, and wherein preferably said one or two phosphorothioate internucleoside linkages are at the 3′end of said second oligonucleotide.

Embodiment 44: The compound of any one of the preceding embodiments, wherein each sugar moiety of said second oligonucleotide is an unmodified DNA sugar moiety, wherein at most two of all internucleoside linkages of said second oligonucleotide are modified internucleoside linkages, and wherein preferably said one or two modified internucleoside linkages are at the 3′end of said second oligonucleotide.

Embodiment 45: The compound of any one of the preceding embodiments, wherein each sugar moiety of said second oligonucleotide is an unmodified DNA sugar moiety, wherein at most one of all internucleoside linkages of said second oligonucleotide is a phosphorothioate internucleoside linkage, and wherein preferably said one phosphorothioate internucleoside linkages is at the 3′end of said second oligonucleotide.

Embodiment 46: The compound of any one of the preceding embodiments, wherein each sugar moiety of said second oligonucleotide is an unmodified DNA sugar moiety, wherein at most one of all internucleoside linkages of said second oligonucleotide is a modified internucleoside linkage, and wherein preferably said one modified internucleoside linkage is at the 3′end of said second oligonucleotide.

Embodiment 47: The compound of any one of the preceding embodiments, wherein each nucleobase of said second oligonucleotide is an unmodified nucleobase.

Embodiment 48: The compound of any one of the preceding embodiments, wherein said second oligonucleotide is a modified oligonucleotide.

Embodiment 49: The compound of any one of the preceding embodiments, wherein said second oligonucleotide comprises at least one modified nucleoside, wherein said modified nucleoside comprises a modified sugar moiety selected from the group consisting of (i) a non-bicyclic modified sugar moiety comprising a furanosyl ring with one or more acyclic substituent at the 2′, 4′, and/or 5′ position, (ii) a bicyclic sugar moiety, (iii) a tricyclic sugar moiety and (iv) a sugar surrogate.

Embodiment 50: The compound of any one of the preceding embodiments, wherein said second oligonucleotide comprises at least one modified nucleoside, wherein said modified nucleoside comprises a non-bicyclic modified sugar moiety comprising a furanosyl ring with an acyclic substituent at the 2′ position. In a preferred embodiment, said acyclic substituent at the 2′ position is selected from 2′-F, 2′-OH, 2′-propargyl, 2′-O-propylamino, 2′-NH₂, 2′-OCH₃ (“OMe” or “O-methyl”), and 2′-O(CH₂)₂OCH₃ (“MOE”).

Embodiment 51: The compound of any one of the preceding embodiments, wherein said second oligonucleotide comprises at least one modified nucleoside, and wherein said modified nucleoside comprises a non-bicyclic modified sugar moiety comprising a furanosyl ring with an acyclic substituent at the 2′ position, wherein said acyclic substituent at the 2′ position is 2′-O(CH₂)₂OCH₃ (“MOE”).

Embodiment 52: The compound of any one of the preceding embodiments, wherein said second oligonucleotide comprises at least one modified nucleoside, and wherein said modified nucleoside comprises a non-bicyclic modified sugar moiety comprising a furanosyl ring with an acyclic substituent at the 2′ position, wherein said acyclic substituent at the 2′ position is 2′-OCH₃ (“OMe” or “O-methyl”).

Embodiment 53: The compound of any one of the preceding embodiments, wherein said second oligonucleotide comprises at least one modified nucleoside, wherein said modified nucleoside comprises a bicyclic sugar moiety, wherein said bicyclic sugar moiety comprises a bridge between the 4′ and the 3′ furanose ring atoms or a bridge between the 4′ and the 2′ furanose ring atoms.

Embodiment 54: The compound of any one of the preceding embodiments, wherein said second oligonucleotide comprises at least one modified nucleoside, wherein said modified nucleoside comprises a tricyclic sugar moiety.

Embodiment 55: The compound of any one of the preceding embodiments, wherein said second oligonucleotide comprises at least one modified nucleoside, wherein said modified nucleoside comprises a sugar surrogate.

Embodiment 56: The compound of any one of the preceding embodiments, wherein said second oligonucleotide comprises at least one modified nucleoside, wherein said modified nucleoside comprises a sugar surrogate, and wherein said sugar surrogate is a morpholino.

Embodiment 57: The compound of any one of the preceding embodiments, wherein said second oligonucleotide comprises at least one unmodified nucleoside and at least one modified nucleoside, wherein said at least one unmodified nucleoside is an unmodified DNA nucleoside, and wherein said modified nucleoside comprises a modified sugar moiety selected from the group consisting of (i) a non-bicyclic modified sugar moiety comprising a furanosyl ring with one or more acyclic substituent at the 2′, 4′, and/or 5′ position, (ii) a bicyclic sugar moiety, (iii) a tricyclic sugar moiety and (iv) a sugar surrogate.

Embodiment 58: The compound of any one of the preceding embodiments, wherein said second oligonucleotide comprises at least one unmodified nucleoside and at least one modified nucleoside, wherein said at least one unmodified nucleoside is selected from the group consisting of a 2′-MOE nucleoside, 2′-OMe nucleoside, an abc-DNA nucleoside, a LNA, a cET, cMOE, a CeNA, a HNA, a ANA, a FANA, a TNA, a tcDNA, a PNA, a GNA, a morpholino or a modified morpholino.

Embodiment 59: The compound of any one of the preceding embodiments, wherein said second oligonucleotide comprises at least one unmodified nucleoside and at least one modified nucleoside, wherein said at least one unmodified nucleoside is an unmodified DNA nucleoside, and wherein said modified nucleoside comprises a non-bicyclic modified sugar moiety comprising a furanosyl ring with an acyclic substituent at the 2′ position.

Embodiment 60: The compound of any one of the preceding embodiments, wherein said acyclic substituent at the 2′ position is selected from 2′-F, 2′-OH, 2′-propargyl, 2′-O-propylamino, 2′-NH₂, 2′-OCH₃ (“OMe” or “O-methyl”), and 2′-O(CH₂)₂OCH₃ (“MOE”).

Embodiment 61: The compound of any one of the preceding embodiments, wherein said second oligonucleotide comprises at least one unmodified nucleoside and at least one modified nucleoside, wherein said at least one unmodified nucleoside is an unmodified DNA nucleoside, and wherein said modified nucleoside comprises a non-bicyclic modified sugar moiety comprising a furanosyl ring with an acyclic substituent at the 2′ position, wherein said acyclic substituent at the 2′ position is 2′-O(CH₂)₂OCH₃ (“MOE”).

Embodiment 62: The compound of any one of the preceding embodiments, wherein said second oligonucleotide comprises at least one unmodified nucleoside and at least one modified nucleoside, wherein said at least one unmodified nucleoside is an unmodified DNA nucleoside, and wherein said modified nucleoside comprises a non-bicyclic modified sugar moiety comprising a furanosyl ring with an acyclic substituent at the 2′ position, wherein said acyclic substituent at the 2′ position is 2′-OCH₃ (“OMe” or “O-methyl”).

Embodiment 63: The compound of any one of the preceding embodiments, wherein said second oligonucleotide comprises at least one unmodified nucleoside and at least one modified nucleoside, wherein said at least one unmodified nucleoside is an unmodified DNA nucleoside, and wherein said modified nucleoside a bicyclic sugar moiety, wherein said bicyclic sugar moiety comprises a bridge between the 4′ and the 3′ furanose ring atoms or a bridge between the 4′ and the 2′ furanose ring atoms.

Embodiment 64: The compound of any one of the preceding embodiments, wherein said second oligonucleotide comprises at least one unmodified nucleoside and at least one modified nucleoside, wherein said at least one unmodified nucleoside is an unmodified DNA nucleoside, and wherein said modified nucleoside is selected from the group consisting of a 2′-MOE nucleoside, 2′-OMe nucleoside, an abc-DNA nucleoside, a LNA, a cET, cMOE, a CeNA, a HNA, a ANA, a FANA, a TNA, a tcDNA, a PNA, a GNA, a morpholino or a modified morpholino.

Embodiment 65: The compound of any one of the preceding embodiments, wherein said second oligonucleotide comprises at least one unmodified nucleoside and at least one modified nucleoside, wherein said at least one unmodified nucleoside is an unmodified DNA nucleoside, and wherein said modified nucleoside a tricyclic sugar moiety.

Embodiment 66: The compound of any one of the preceding embodiments, wherein said second oligonucleotide comprises at least one unmodified nucleoside and at least one modified nucleoside, wherein said at least one unmodified nucleoside is an unmodified DNA nucleoside, and wherein said modified nucleoside is a sugar surrogate.

Embodiment 67: The compound of any one of the preceding embodiments, wherein said second oligonucleotide comprises at least one unmodified nucleoside and at least one modified nucleoside, wherein said at least one unmodified nucleoside is an unmodified DNA nucleoside, and wherein said modified nucleoside is a sugar surrogate, and wherein said sugar surrogate is a morpholino.

Embodiment 68: The compound of any one of the preceding embodiments, wherein said second oligonucleotide is a modified second oligonucleotide comprising unmodified nucleosides and at least one but at most four modified nucleosides, wherein said unmodified nucleosides are unmodified DNA nucleosides, and wherein said modified nucleoside comprises a modified sugar moiety selected from the group consisting of (i) a non-bicyclic modified sugar moiety comprising a furanosyl ring with one or more acyclic substituent at the 2′, 4′, and/or 5′ position, (ii) a bicyclic sugar moiety, (iii) a tricyclic sugar moiety and (iv) a sugar surrogate.

Embodiment 69: The compound of any one of the preceding embodiments, wherein said second oligonucleotide is a modified second oligonucleotide comprising unmodified nucleosides and at least one but at most four modified nucleosides, wherein said unmodified nucleosides are unmodified DNA nucleosides, and wherein said modified nucleoside comprises a non-bicyclic modified sugar moiety comprising a furanosyl ring with an acyclic substituent at the 2′ position. In a preferred embodiment, said acyclic substituent at the 2′ position is selected from 2′-F, 2′-OH, 2′-propargyl, 2′-O-propylamino, 2′-NH₂, 2′-OCH₃ (“OMe” or “O-methyl”), and 2′-O(CH₂)₂OCH₃ (“MOE”).

Embodiment 70: The compound of any one of the preceding embodiments, wherein said second oligonucleotide is a modified second oligonucleotide comprising unmodified nucleosides and at least one but at most four modified nucleosides, wherein said unmodified nucleosides are unmodified DNA nucleosides, and wherein said modified nucleoside comprises a non-bicyclic modified sugar moiety comprising a furanosyl ring with an acyclic substituent at the 2′ position, wherein said acyclic substituent at the 2′ position is 2′-O(CH₂)₂OCH₃ (“MOE”).

Embodiment 71: The compound of any one of the preceding embodiments, wherein said second oligonucleotide is a modified second oligonucleotide comprising unmodified nucleosides and at least one but at most four modified nucleosides, wherein said unmodified nucleosides are unmodified DNA nucleosides, and wherein said modified nucleoside comprises a non-bicyclic modified sugar moiety comprising a furanosyl ring with an acyclic substituent at the 2′ position, wherein said acyclic substituent at the 2′ position is 2′-OCH₃ (“OMe” or “O-methyl”).

Embodiment 72: The compound of any one of the preceding embodiments, wherein said second oligonucleotide is a modified second oligonucleotide comprising unmodified nucleosides and at least one but at most four modified nucleosides, wherein said unmodified nucleosides are unmodified DNA nucleosides, and wherein said modified nucleoside a bicyclic sugar moiety, wherein said bicyclic sugar moiety comprises a bridge between the 4′ and the 3′ furanose ring atoms or a bridge between the 4′ and the 2′ furanose ring atoms.

Embodiment 73: The compound of any one of the preceding embodiments, wherein said second oligonucleotide is a modified second oligonucleotide comprising unmodified nucleosides and at least one but at most four modified nucleosides, wherein said unmodified nucleosides are unmodified DNA nucleosides, and wherein said modified nucleoside is selected from the group consisting of a 2′-MOE nucleoside, 2′-OMe nucleoside, an abc-DNA nucleoside, a LNA, a cET, cMOE, a CeNA, a HNA, a ANA, a FANA, a TNA, a tcDNA, a PNA, a GNA, a morpholino or a modified morpholino.

Embodiment 74: The compound of any one of the preceding embodiments, wherein said second oligonucleotide is a modified second oligonucleotide comprising unmodified nucleosides and at least one but at most three modified nucleosides, wherein said unmodified nucleosides are unmodified DNA nucleosides, and wherein said modified nucleoside is selected from the group consisting of a 2′-MOE nucleoside, 2′-OMe nucleoside, an abc-DNA nucleoside, a LNA, a cET, a HNA, a ANA, a FANA, a tcDNA, a PNA, a morpholino or a modified morpholino.

Embodiment 75: The compound of any one of the preceding embodiments, wherein said second oligonucleotide is a modified second oligonucleotide comprising unmodified nucleosides and at least one but at most two modified nucleosides, wherein said unmodified nucleosides are unmodified DNA nucleosides, and wherein said modified nucleoside is selected from the group consisting of a 2′-MOE nucleoside, 2′-OMe nucleoside, an abc-DNA nucleoside, a LNA, a cET, a HNA, a ANA, a FANA, a tcDNA and a morpholino.

Embodiment 76: The compound of any one of the preceding embodiments, wherein said second oligonucleotide is a modified second oligonucleotide comprising unmodified nucleosides and at least one but at most one modified nucleosides, wherein said unmodified nucleosides are unmodified DNA nucleosides, and wherein said modified nucleoside is selected from the group consisting of a 2′-MOE nucleoside, 2′-OMe nucleoside, an abc-DNA nucleoside, a LNA, a cET, a ANA, a FANA, and a morpholino.

Embodiment 77: The compound of any one of the preceding embodiments, wherein said second oligonucleotide is a modified second oligonucleotide comprising unmodified nucleosides and at least one but at most four modified nucleosides, wherein said unmodified nucleosides are unmodified DNA nucleosides, and wherein said modified nucleoside a tricyclic sugar moiety.

Embodiment 78: The compound of any one of the preceding embodiments, wherein said second oligonucleotide is a modified second oligonucleotide comprising unmodified nucleosides and at least one but at most four modified nucleosides, wherein said unmodified nucleosides are unmodified DNA nucleosides, and wherein said modified nucleoside is a sugar surrogate.

Embodiment 79: The compound of any one of the preceding embodiments, wherein said second oligonucleotide is a modified second oligonucleotide comprising unmodified nucleosides and at least one but at most four modified nucleosides, wherein said unmodified nucleosides are unmodified DNA nucleosides, and wherein said modified nucleoside is a sugar surrogate, and wherein said sugar surrogate is a morpholino.

Embodiment 80: The compound of any one of the preceding embodiments, wherein each nucleoside of said second oligonucleotide comprises a modified nucleoside, wherein said modified nucleoside comprises a modified sugar moiety selected from the group consisting of (i) a non-bicyclic modified sugar moiety comprising a furanosyl ring with one or more acyclic substituent at the 2′, 4′, and/or 5′ position, (ii) a bicyclic sugar moiety, (iii) a tricyclic sugar moiety and (iv) a sugar surrogate.

Embodiment 81: The compound of any one of the preceding embodiments, wherein each nucleoside of said second oligonucleotide comprises at least one modified nucleoside, wherein said modified nucleoside comprises a non-bicyclic modified sugar moiety comprising a furanosyl ring with an acyclic substituent at the 2′ position. In a preferred embodiment, said acyclic substituent at the 2′ position is selected from 2′-F, 2′-OH, 2′-propargyl, 2′-O-propylamino, 2′-NH₂, 2′-OCH₃ (“OMe” or “O-methyl”), and 2′-O(CH₂)₂OCH₃ (“MOE”).

Embodiment 82: The compound of any one of the preceding embodiments, wherein each nucleoside of said second oligonucleotide comprises a non-bicyclic modified sugar moiety comprising a furanosyl ring with an acyclic substituent at the 2′ position, wherein said acyclic substituent at the 2′ position is 2′-O(CH₂)₂OCH₃ (“MOE”).

Embodiment 83: The compound of any one of the preceding embodiments, wherein each nucleoside of said second oligonucleotide comprises a non-bicyclic modified sugar moiety comprising a furanosyl ring with an acyclic substituent at the 2′ position, wherein said acyclic substituent at the 2′ position is 2′-OCH₃ (“OMe” or “O-methyl”).

Embodiment 84: The compound of any one of the preceding embodiments, wherein each nucleoside of said second oligonucleotide comprises a bicyclic sugar moiety, wherein said bicyclic sugar moiety comprises a bridge between the 4′ and the 3′ furanose ring atoms or a bridge between the 4′ and the 2′ furanose ring atoms.

Embodiment 85: The compound of any one of the preceding embodiments, wherein each nucleoside of said second oligonucleotide comprises a tricyclic sugar moiety.

Embodiment 86: The compound of any one of the preceding embodiments, wherein each nucleoside of said second oligonucleotide comprises a sugar surrogate.

Embodiment 87: The compound of any one of the preceding embodiments, wherein each nucleoside of said second oligonucleotide comprises a sugar surrogate, and wherein said sugar surrogate is a morpholino.

Embodiment 88: The compound of any one of the preceding embodiments, wherein said second oligonucleotide is a modified oligonucleotide, and wherein said modified second oligonucleotide comprises at least one modified nucleoside, wherein said modified nucleoside is selected from the group consisting of a 2′-MOE nucleoside, 2′-OMe nucleoside, an abc-DNA nucleoside, a LNA, a cET, cMOE, a CeNA, a HNA, a ANA, a FANA, a TNA, a tcDNA, a PNA, a GNA, a morpholino or a modified morpholino.

Embodiment 89: The compound of any one of the preceding embodiments, wherein said second oligonucleotide comprises at least one modified nucleoside, wherein said modified nucleoside is selected from the group consisting of a 2′-MOE nucleoside, 2′-OMe nucleoside, an abc-DNA nucleoside, a LNA, a CeNA, a HNA, a ANA, a FANA, a TNA, a tcDNA and a morpholino.

Embodiment 90: The compound of any one of the preceding embodiments, wherein said second oligonucleotide comprises at least one modified nucleoside, wherein said modified nucleoside comprises a 2′-OMe sugar moiety.

Embodiment 91: The compound of any one of the preceding embodiments, wherein said second oligonucleotide comprises at least one modified nucleoside, wherein said modified nucleoside comprises a 2′-MOE sugar moiety.

Embodiment 92: The compound of any one of the preceding embodiments, wherein said second oligonucleotide comprises at least one modified nucleoside, wherein said modified nucleoside is a 2′-MOE nucleoside,

Embodiment 93: The compound of any one of the preceding embodiments, wherein said second oligonucleotide comprises at least one modified nucleoside, wherein said modified nucleoside is an abc-DNA nucleoside.

Embodiment 94: The compound of any one of the preceding embodiments, wherein said second oligonucleotide comprises at least one modified nucleoside, wherein said modified nucleoside is a LNA.

Embodiment 95: The compound of any one of the preceding embodiments, wherein said second oligonucleotide comprises at least one modified nucleoside, wherein said modified nucleoside is a CeNA.

Embodiment 96: The compound of any one of the preceding embodiments, wherein said second oligonucleotide comprises at least one modified nucleoside, wherein said modified nucleoside is a HNA.

Embodiment 97: The compound of any one of the preceding embodiments, wherein said second oligonucleotide comprises at least one modified nucleoside, wherein said modified nucleoside is an ANA.

Embodiment 98: The compound of any one of the preceding embodiments, wherein said second oligonucleotide comprises at least one modified nucleoside, wherein said modified nucleoside is a FANA.

Embodiment 99: The compound of any one of the preceding embodiments, wherein said second oligonucleotide comprises at least one modified nucleoside, wherein said modified nucleoside is a TNA.

Embodiment 100: The compound of any one of the preceding embodiments, wherein said second oligonucleotide comprises at least one modified nucleoside, wherein said modified nucleoside is a tcDNA.

Embodiment 101: The compound of any one of the preceding embodiments, wherein said second oligonucleotide comprises at least one modified nucleoside, wherein said modified nucleoside is a morpholino.

Embodiment 102: The compound of any one of the preceding embodiments, wherein said second oligonucleotide comprises at least one modified nucleoside, wherein said modified nucleoside is a modified morpholino.

Embodiment 103: The compound of any one of the preceding embodiments, wherein said second oligonucleotide is a modified oligonucleotide, and wherein said modified second oligonucleotide comprises at least one unmodified nucleoside and at least one modified nucleoside, wherein said at least one unmodified nucleoside is unmodified DNA sugar moiety, and wherein said modified nucleoside is selected from the group consisting of a 2′-MOE nucleoside, 2′-OMe nucleoside, an abc-DNA nucleoside, a LNA, a cET, cMOE, a CeNA, a HNA, a ANA, a FANA, a TNA, a tcDNA, a PNA, a GNA, a morpholino or a modified morpholino.

Embodiment 104: The compound of any one of the preceding embodiments, wherein said second oligonucleotide comprises at least one unmodified nucleoside and at least one modified nucleoside, wherein said at least one unmodified nucleoside is unmodified DNA sugar moiety, and wherein said modified nucleoside is selected from the group consisting of an a 2′-MOE nucleoside, abc-DNA nucleoside, a LNA, a CeNA, a HNA, a ANA, a FANA, a TNA, a tcDNA and a morpholino.

Embodiment 105: The compound of any one of the preceding embodiments, wherein said second oligonucleotide comprises at least one unmodified nucleoside and at least one modified nucleoside, wherein said at least one unmodified nucleoside is unmodified DNA sugar moiety, and wherein said modified nucleoside comprises a 2′-OMe sugar moiety.

Embodiment 106: The compound of any one of the preceding embodiments, wherein said second oligonucleotide comprises at least one unmodified nucleoside and at least one modified nucleoside, wherein said at least one unmodified nucleoside is unmodified DNA sugar moiety, and wherein said modified nucleoside comprises a 2′-MOE sugar moiety.

Embodiment 107: The compound of any one of the preceding embodiments, wherein said second oligonucleotide comprises at least one unmodified nucleoside and at least one modified nucleoside, wherein said at least one unmodified nucleoside is unmodified DNA sugar moiety, and wherein said modified nucleoside is a 2′-MOE nucleoside.

Embodiment 108: The compound of any one of the preceding embodiments, wherein said second oligonucleotide comprises at least one unmodified nucleoside and at least one modified nucleoside, wherein said at least one unmodified nucleoside is unmodified DNA sugar moiety, and wherein said modified nucleoside is an abc-DNA nucleoside.

Embodiment 109: The compound of any one of the preceding embodiments, wherein said second oligonucleotide comprises at least one unmodified nucleoside and at least one modified nucleoside, wherein said at least one unmodified nucleoside is unmodified DNA sugar moiety, and wherein said modified nucleoside is a LNA.

Embodiment 110: The compound of any one of the preceding embodiments, wherein said second oligonucleotide comprises at least one unmodified nucleoside and at least one modified nucleoside, wherein said at least one unmodified nucleoside is unmodified DNA sugar moiety, and wherein said modified nucleoside is a CeNA.

Embodiment 111: The compound of any one of the preceding embodiments, wherein said second oligonucleotide comprises at least one unmodified nucleoside and at least one modified nucleoside, wherein said at least one unmodified nucleoside is unmodified DNA sugar moiety, and wherein said modified nucleoside is a HNA.

Embodiment 112: The compound of any one of the preceding embodiments, wherein said second oligonucleotide comprises at least one unmodified nucleoside and at least one modified nucleoside, wherein said at least one unmodified nucleoside is unmodified DNA sugar moiety, and wherein said modified nucleoside is an ANA.

Embodiment 113: The compound of any one of the preceding embodiments, wherein said second oligonucleotide comprises at least one unmodified nucleoside and at least one modified nucleoside, wherein said at least one unmodified nucleoside is unmodified DNA sugar moiety, and wherein said modified nucleoside is a FANA.

Embodiment 114: The compound of any one of the preceding embodiments, wherein said second oligonucleotide comprises at least one unmodified nucleoside and at least one modified nucleoside, wherein said at least one unmodified nucleoside is unmodified DNA sugar moiety, and wherein said modified nucleoside is a TNA.

Embodiment 115: The compound of any one of the preceding embodiments, wherein said second oligonucleotide comprises at least one unmodified nucleoside and at least one modified nucleoside, wherein said at least one unmodified nucleoside is unmodified DNA sugar moiety, and wherein said modified nucleoside is a tcDNA.

Embodiment 116: The compound of any one of the preceding embodiments, wherein said second oligonucleotide comprises at least one unmodified nucleoside and at least one modified nucleoside, wherein said at least one unmodified nucleoside is unmodified DNA sugar moiety, and wherein said modified nucleoside is a morpholino.

Embodiment 117: The compound of any one of the preceding embodiments, wherein said second oligonucleotide comprises at least one unmodified nucleoside and at least one modified nucleoside, wherein said at least one unmodified nucleoside is unmodified DNA sugar moiety, and wherein said modified nucleoside is a modified morpholino.

Embodiment 118: The compound of any one of the preceding embodiments, wherein said second oligonucleotide is a modified second oligonucleotide comprising unmodified nucleosides and at least one but at most four modified nucleosides, wherein said unmodified nucleosides are unmodified DNA nucleosides, and wherein said modified nucleoside is selected from the group consisting of a 2′-MOE nucleoside, 2′-OMe nucleoside, an abc-DNA nucleoside, a LNA, a cET, cMOE, a CeNA, a HNA, a ANA, a FANA, a TNA, a tcDNA, a PNA, a GNA, a morpholino or a modified morpholino, wherein preferably said modified nucleosides are positioned at the 5′-end and/or at the 3′-end of said second oligonucleotide.

Embodiment 119: The compound of any one of the preceding embodiments, wherein said second oligonucleotide is a modified second oligonucleotide comprising unmodified nucleosides and at least one but at most four modified nucleosides, wherein said unmodified nucleosides are unmodified DNA nucleosides, and wherein said modified nucleoside is selected from the group consisting of a 2′-MOE nucleoside, 2′-OMe nucleoside, an abc-DNA nucleoside, a LNA, a CeNA, a HNA, a ANA, a FANA, a TNA, a tcDNA and a morpholino, wherein preferably said modified nucleosides are positioned at the 5′-end and/or at the 3′-end of said second oligonucleotide.

Embodiment 120: The compound of any one of the preceding embodiments, wherein said second oligonucleotide is a modified second oligonucleotide comprising unmodified nucleosides and at least one but at most four modified nucleosides, wherein said unmodified nucleosides are unmodified DNA nucleosides, and wherein said modified nucleoside comprises a 2′-OMe sugar moiety, wherein preferably said modified nucleosides are positioned at the 5′-end and/or at the 3′-end of said second oligonucleotide.

Embodiment 121: The compound of any one of the preceding embodiments, wherein said second oligonucleotide is a modified second oligonucleotide comprising unmodified nucleosides and at least one but at most four modified nucleosides, wherein said unmodified nucleosides are unmodified DNA nucleosides, and wherein said modified nucleoside comprises a 2′-MOE sugar moiety, wherein preferably said modified nucleosides are positioned at the 5′-end and/or at the 3′-end of said second oligonucleotide.

Embodiment 122: The compound of any one of the preceding embodiments, wherein said second oligonucleotide is a modified second oligonucleotide comprising unmodified nucleosides and at least one but at most four modified nucleosides, wherein said unmodified nucleosides are unmodified DNA nucleosides, and wherein said modified nucleoside is a 2′-MOE nucleoside, wherein preferably said modified nucleosides are positioned at the 5′-end and/or at the 3′-end of said second oligonucleotide.

Embodiment 123: The compound of any one of the preceding embodiments, wherein said second oligonucleotide is a modified second oligonucleotide comprising unmodified nucleosides and at least one but at most four modified nucleosides, wherein said unmodified nucleosides are unmodified DNA nucleosides, and wherein said modified nucleoside is an abc-DNA nucleoside, wherein preferably said modified nucleosides are positioned at the 5′-end and/or at the 3′-end of said second oligonucleotide.

Embodiment 124: The compound of any one of the preceding embodiments, wherein said second oligonucleotide is a modified second oligonucleotide comprising unmodified nucleosides and at least one but at most four modified nucleosides, wherein said unmodified nucleosides are unmodified DNA nucleosides, and wherein said modified nucleoside is a LNA, wherein preferably said modified nucleosides are positioned at the 5′-end and/or at the 3′-end of said second oligonucleotide.

Embodiment 125: The compound of any one of the preceding embodiments, wherein said second oligonucleotide is a modified second oligonucleotide comprising unmodified nucleosides and at least one but at most four modified nucleosides, wherein said unmodified nucleosides are unmodified DNA nucleosides, and wherein said modified nucleoside is a CeNA, wherein preferably said modified nucleosides are positioned at the 5′-end and/or at the 3′-end of said second oligonucleotide.

Embodiment 126: The compound of any one of the preceding embodiments, wherein said second oligonucleotide is a modified second oligonucleotide comprising unmodified nucleosides and at least one but at most four modified nucleosides, wherein said unmodified nucleosides are unmodified DNA nucleosides, and wherein said modified nucleoside is a HNA, wherein preferably said modified nucleosides are positioned at the 5′-end and/or at the 3′-end of said second oligonucleotide.

Embodiment 127: The compound of any one of the preceding embodiments, wherein said second oligonucleotide is a modified second oligonucleotide comprising unmodified nucleosides and at least one but at most four modified nucleosides, wherein said unmodified nucleosides are unmodified DNA nucleosides, and wherein said modified nucleoside is an ANA, wherein preferably said modified nucleosides are positioned at the 5′-end and/or at the 3′-end of said second oligonucleotide.

Embodiment 128: The compound of any one of the preceding embodiments, wherein said second oligonucleotide is a modified second oligonucleotide comprising unmodified nucleosides and at least one but at most four modified nucleosides, wherein said unmodified nucleosides are unmodified DNA nucleosides, and wherein said modified nucleoside is a FANA, wherein preferably said modified nucleosides are positioned at the 5′-end and/or at the 3′-end of said second oligonucleotide.

Embodiment 129: The compound of any one of the preceding embodiments, wherein said second oligonucleotide is a modified second oligonucleotide comprising unmodified nucleosides and at least one but at most four modified nucleosides, wherein said unmodified nucleosides are unmodified DNA nucleosides, and wherein said modified nucleoside is a TNA, wherein preferably said modified nucleosides are positioned at the 5′-end and/or at the 3′-end of said second oligonucleotide.

Embodiment 130: The compound of any one of the preceding embodiments, wherein said second oligonucleotide is a modified second oligonucleotide comprising unmodified nucleosides and at least one but at most four modified nucleosides, wherein said unmodified nucleosides are unmodified DNA nucleosides, and wherein said modified nucleoside is a tcDNA, wherein preferably said modified nucleosides are positioned at the 5′-end and/or at the 3′-end of said second oligonucleotide.

Embodiment 131: The compound of any one of the preceding embodiments, wherein said second oligonucleotide is a modified second oligonucleotide comprising unmodified nucleosides and at least one but at most four modified nucleosides, wherein said unmodified nucleosides are unmodified DNA nucleosides, and wherein said modified nucleoside is a morpholino, wherein preferably said modified nucleosides are positioned at the 5′-end and/or at the 3′-end of said second oligonucleotide.

Embodiment 132: The compound of any one of the preceding embodiments, wherein said second oligonucleotide is a modified second oligonucleotide comprising unmodified nucleosides and at least one but at most four modified nucleosides, wherein said unmodified nucleosides are unmodified DNA nucleosides, and wherein said modified nucleoside is a modified morpholino, wherein preferably said modified nucleosides are positioned at the 5′-end and/or at the 3′-end of said second oligonucleotide.

Embodiment 133: The compound of any one of the preceding embodiments, wherein said second oligonucleotide is a modified second oligonucleotide comprising unmodified nucleosides and one or two modified nucleosides, wherein said unmodified nucleosides are unmodified DNA nucleosides, and wherein said modified nucleoside is selected from the group consisting of a 2′-MOE nucleoside, 2′-OMe nucleoside, an abc-DNA nucleoside, a LNA, a cET, cMOE, a CeNA, a HNA, a ANA, a FANA, a TNA, a tcDNA, a PNA, a GNA, a morpholino or a modified morpholino, and wherein preferably said one or two modified nucleosides are the nucleosides at the 3′end of said second oligonucleotide.

Embodiment 134: The compound of any one of the preceding embodiments, wherein said second oligonucleotide is a modified second oligonucleotide comprising unmodified nucleosides and one or two modified nucleosides, wherein said unmodified nucleosides are unmodified DNA nucleosides, and wherein said modified nucleoside is selected from the group consisting of a 2′-MOE nucleoside, 2′-OMe nucleoside, an abc-DNA nucleoside, a LNA, a CeNA, a HNA, a ANA, a FANA, a TNA, a tcDNA and a morpholino, and wherein preferably said one or two modified nucleosides are the nucleosides at the 3′end of said second oligonucleotide.

Embodiment 135: The compound of any one of the preceding embodiments, wherein said second oligonucleotide is a modified second oligonucleotide comprising unmodified nucleosides and one or two modified nucleosides, wherein said unmodified nucleosides are unmodified DNA nucleosides, and wherein said modified nucleoside comprises a 2′-OMe sugar moiety, and wherein preferably said one or two modified nucleosides are the nucleosides at the 3′end of said second oligonucleotide.

Embodiment 136: The compound of any one of the preceding embodiments, wherein said second oligonucleotide is a modified second oligonucleotide comprising unmodified nucleosides and one or two modified nucleosides, wherein said unmodified nucleosides are unmodified DNA nucleosides, and wherein said modified nucleoside comprises a 2′-MOE sugar moiety, and wherein preferably said one or two modified nucleosides are the nucleosides at the 3′end of said second oligonucleotide.

Embodiment 137: The compound of any one of the preceding embodiments, wherein said second oligonucleotide is a modified second oligonucleotide comprising unmodified nucleosides and one or two modified nucleosides, wherein said unmodified nucleosides are unmodified DNA nucleosides, and wherein said modified nucleoside is a 2′-MOE nucleoside, and wherein preferably said one or two modified nucleosides are the nucleosides at the 3′end of said second oligonucleotide.

Embodiment 138: The compound of any one of the preceding embodiments, wherein said second oligonucleotide is a modified second oligonucleotide comprising unmodified nucleosides and one or two modified nucleosides, wherein said unmodified nucleosides are unmodified DNA nucleosides, and wherein said modified nucleoside is an abc-DNA nucleoside, and wherein preferably said one or two modified nucleosides are the nucleosides at the 3′end of said second oligonucleotide.

Embodiment 139: The compound of any one of the preceding embodiments, wherein said second oligonucleotide is a modified second oligonucleotide comprising unmodified nucleosides and one or two modified nucleosides, wherein said unmodified nucleosides are unmodified DNA nucleosides, and wherein said modified nucleoside is a LNA, and wherein preferably said one or two modified nucleosides are the nucleosides at the 3′end of said second oligonucleotide.

Embodiment 140: The compound of any one of the preceding embodiments, wherein said second oligonucleotide is a modified second oligonucleotide comprising unmodified nucleosides and one or two modified nucleosides, wherein said unmodified nucleosides are unmodified DNA nucleosides, and wherein said modified nucleoside is a CeNA, and wherein preferably said one or two modified nucleosides are the nucleosides at the 3′end of said second oligonucleotide.

Embodiment 141: The compound of any one of the preceding embodiments, wherein said second oligonucleotide is a modified second oligonucleotide comprising unmodified nucleosides and one or two modified nucleosides, wherein said unmodified nucleosides are unmodified DNA nucleosides, and wherein said modified nucleoside is a HNA, and wherein preferably said one or two modified nucleosides are the nucleosides at the 3′end of said second oligonucleotide.

Embodiment 142: The compound of any one of the preceding embodiments, wherein said second oligonucleotide is a modified second oligonucleotide comprising unmodified nucleosides and one or two modified nucleosides, wherein said unmodified nucleosides are unmodified DNA nucleosides, and wherein said modified nucleoside is an ANA, and wherein preferably said one or two modified nucleosides are the nucleosides at the 3′end of said second oligonucleotide.

Embodiment 143: The compound of any one of the preceding embodiments, wherein said second oligonucleotide is a modified second oligonucleotide comprising unmodified nucleosides and one or two modified nucleosides, wherein said unmodified nucleosides are unmodified DNA nucleosides, and wherein said modified nucleoside is a FANA, and wherein preferably said one or two modified nucleosides are the nucleosides at the 3′end of said second oligonucleotide.

Embodiment 144: The compound of any one of the preceding embodiments, wherein said second oligonucleotide is a modified second oligonucleotide comprising unmodified nucleosides and one or two modified nucleosides, wherein said unmodified nucleosides are unmodified DNA nucleosides, and wherein said modified nucleoside is a TNA, and wherein preferably said one or two modified nucleosides are the nucleosides at the 3′end of said second oligonucleotide.

Embodiment 145: The compound of any one of the preceding embodiments, wherein said second oligonucleotide is a modified second oligonucleotide comprising unmodified nucleosides and one or two modified nucleosides, wherein said unmodified nucleosides are unmodified DNA nucleosides, and wherein said modified nucleoside is a tcDNA, and wherein preferably said one or two modified nucleosides are the nucleosides at the 3′end of said second oligonucleotide.

Embodiment 146: The compound of any one of the preceding embodiments, wherein said second oligonucleotide is a modified second oligonucleotide comprising unmodified nucleosides and one or two modified nucleosides, wherein said unmodified nucleosides are unmodified DNA nucleosides, and wherein said modified nucleoside is a morpholino, and wherein preferably said one or two modified nucleosides are the nucleosides at the 3′end of said second oligonucleotide.

Embodiment 147: The compound of any one of the preceding embodiments, wherein said second oligonucleotide is a modified second oligonucleotide comprising unmodified nucleosides and one or two modified nucleosides, wherein said unmodified nucleosides are unmodified DNA nucleosides, and wherein said modified nucleoside is a modified morpholino, and wherein preferably said one or two modified nucleosides are the nucleosides at the 3′end of said second oligonucleotide.

Embodiment 148: The compound of any one of the preceding embodiments, wherein each nucleoside of said second oligonucleotide is selected from the group consisting of a 2′-MOE nucleoside, 2′-OMe nucleoside, an abc-DNA nucleoside, a LNA, a cET, cMOE, a CeNA, a HNA, a ANA, a FANA, a TNA, a tcDNA, a PNA, a GNA, a morpholino or a modified morpholino.

Embodiment 149: The compound of any one of the preceding embodiments, wherein each nucleoside of said second oligonucleotide is selected from the group consisting of a 2′-MOE nucleoside, 2′-OMe nucleoside, an abc-DNA nucleoside, a LNA, a cET, cMOE, a CeNA, a HNA, a ANA, a FANA, a TNA, a tcDNA, a PNA, a GNA, a morpholino or a modified morpholino.

Embodiment 150: The compound of any one of the preceding embodiments, wherein each nucleoside of said second oligonucleotide is selected from the group consisting of a 2′-MOE nucleoside, 2′-OMe nucleoside, an abc-DNA nucleoside, a LNA, a CeNA, a HNA, a ANA, a FANA, a TNA, a tcDNA and a morpholino.

Embodiment 151: The compound of any one of the preceding embodiments, wherein each nucleoside of said second oligonucleotide comprises a 2′-OMe sugar moiety.

Embodiment 152: The compound of any one of the preceding embodiments, wherein each nucleoside of said second oligonucleotide comprises a 2′-MOE sugar moiety.

Embodiment 153: The compound of any one of the preceding embodiments, wherein each nucleoside of said second oligonucleotide is a 2′-MOE nucleoside.

Embodiment 154: The compound of any one of the preceding embodiments, wherein each nucleoside of said second oligonucleotide is an abc-DNA nucleoside.

Embodiment 155: The compound of any one of the preceding embodiments, wherein each nucleoside of said second oligonucleotide is a LNA.

Embodiment 156: The compound of any one of the preceding embodiments, wherein each nucleoside of said second oligonucleotide is a CeNA.

Embodiment 157: The compound of any one of the preceding embodiments, wherein each nucleoside of said second oligonucleotide is a HNA.

Embodiment 158: The compound of any one of the preceding embodiments, wherein each nucleoside of said second oligonucleotide is an ANA.

Embodiment 159: The compound of any one of the preceding embodiments, wherein each nucleoside of said second oligonucleotide is a FANA.

Embodiment 160: The compound of any one of the preceding embodiments, wherein each nucleoside of said second oligonucleotide is a TNA.

Embodiment 161: The compound of any one of the preceding embodiments, wherein each nucleoside of said second oligonucleotide is a tcDNA.

Embodiment 162: The compound of any one of the preceding embodiments, wherein each nucleoside of said second oligonucleotide is a morpholino.

Embodiment 163: The compound of any one of the preceding embodiments, wherein each nucleoside of said second oligonucleotide is a modified morpholino.

Embodiment 164: The compound of any one of the preceding embodiments, wherein said second oligonucleotide is a modified oligonucleotide, and wherein said modified second oligonucleotide is a uniformly modified oligonucleotide.

Embodiment 165: The compound of any one of the preceding embodiments, wherein said second oligonucleotide is a modified oligonucleotide, and wherein each nucleobase of said modified second oligonucleotide is an unmodified nucleobase.

Embodiment 166: The compound of any one of the preceding embodiments, wherein the internucleoside linkages of said first oligonucleotide are essentially free of phosphorothioate internucleoside linkages.

Embodiment 167: The compound of any one of the preceding embodiments, wherein the internucleoside linkages of said first oligonucleotide are essentially free of modified internucleoside linkages.

Embodiment 168: The compound of any one of the preceding embodiments, wherein essentially all internucleoside linkages of said first oligonucleotide are unmodified phosphodiester internucleoside linkages.

Embodiment 169: The compound of any one of the preceding embodiments, wherein the percentage of modified internucleoside linkages relative to the total number of internucleoside linkages of said first oligonucleotide is no more than no more than 40%, preferably no more than 35%, further preferably no more no more than 30%.

Embodiment 170: The compound of any one of the preceding embodiments, wherein the percentage of phosphorothioate internucleoside linkages relative to the total number of internucleoside linkages of said first oligonucleotide is no more than no more than 40%, preferably no more than 35%, further preferably no more no more than 30%.

Embodiment 171: The compound of any one of the preceding embodiments, wherein the percentage of modified internucleoside linkages relative to the total number of internucleoside linkages of said first oligonucleotide is no more than no more than 25%, preferably no more than 20%, further preferably no more no more than 15%.

Embodiment 172: The compound of any one of the preceding embodiments, wherein the percentage of phosphorothioate internucleoside linkages relative to the total number of internucleoside linkages of said first oligonucleotide is no more than no more than 25%, preferably no more than 20%, further preferably no more no more than 15%.

Embodiment 173: The compound of any one of the preceding embodiments, wherein the percentage of modified internucleoside linkages relative to the total number of internucleoside linkages of said first oligonucleotide is no more than no more than 12%, preferably no more than 10%, further preferably no more no more than 8%.

Embodiment 174: The compound of any one of the preceding embodiments, wherein the percentage of phosphorothioate internucleoside linkages relative to the total number of internucleoside linkages of said first oligonucleotide is no more than no more than 12%, preferably no more than 10%, further preferably no more no more than 8%.

Embodiment 175: The compound of any one of the preceding embodiments, wherein at most five, preferably at most four of all internucleoside linkages of said first oligonucleotide are phosphorothioate internucleoside linkages.

Embodiment 176: The compound of any one of the preceding embodiments, wherein at most five, preferably at most four of all internucleoside linkages of said first oligonucleotide are modified internucleoside linkages.

Embodiment 177: The compound of any one of the preceding embodiments, wherein at most three of all internucleoside linkages of said first oligonucleotide are phosphorothioate internucleoside linkages.

Embodiment 178: The compound of any one of the preceding embodiments, wherein at most three of all internucleoside linkages of said first oligonucleotide are modified internucleoside linkages.

Embodiment 179: The compound of any one of the preceding embodiments, wherein at most two of all internucleoside linkages of said first oligonucleotide are phosphorothioate internucleoside linkages.

Embodiment 180: The compound of any one of the preceding embodiments, wherein at most one of all internucleoside linkages of said first oligonucleotide are modified internucleoside linkages.

Embodiment 181: The compound of any one of the preceding embodiments, wherein at none of all internucleoside linkages of said first oligonucleotide is a phosphorothioate internucleoside linkage.

Embodiment 182: The compound of any one of the preceding embodiments, wherein at most one of all internucleoside linkages of said first oligonucleotide is a modified internucleoside linkage.

Embodiment 183: The compound of any one of the preceding embodiments, wherein the percentage of unmodified phosphodiester internucleoside linkages relative to the total number of internucleoside linkages of said first oligonucleotide is at least 60%, preferably at least 65%, further preferably at least 70%.

Embodiment 184: The compound of any one of the preceding embodiments, wherein the percentage of unmodified phosphodiester internucleoside linkages relative to the total number of internucleoside linkages of said first oligonucleotide is at least 75%, preferably at least 80%, further preferably at least 85%.

Embodiment 185: The compound of any one of the preceding embodiments, wherein the percentage of unmodified phosphodiester internucleoside linkages relative to the total number of internucleoside linkages of said first oligonucleotide is at least 88%, preferably at least 90%, further preferably at least 92%.

Embodiment 186: The compound of any one of the preceding embodiments, wherein all internucleoside linkages of said first oligonucleotide are unmodified phosphodiester internucleoside linkages.

Embodiment 187: The compound of any one of the preceding embodiments, wherein essentially all internucleoside linkages of said first oligonucleotide are selected from the group consisting of unmodified phosphodiester internucleoside linkages and phosphorodiamidate internucleoside linkages.

Embodiment 188: The compound of any one of the preceding embodiments, wherein the percentage of internucleoside linkages which are unmodified phosphodiester and phosphorodiamidate internucleoside linkages relative to the total number of internucleoside linkages of said first oligonucleotide is at least 60%, preferably at least 65%, further preferably at least 70%.

Embodiment 189: The compound of any one of the preceding embodiments, wherein the percentage of internucleoside linkages which are unmodified phosphodiester and phosphorodiamidate internucleoside linkages relative to the total number of internucleoside linkages of said first oligonucleotide is at least 75%, preferably at least 80%, further preferably at least 85%.

Embodiment 190: The compound of any one of the preceding embodiments, wherein the percentage of internucleoside linkages which are unmodified phosphodiester and phosphorodiamidate internucleoside linkages relative to the total number of internucleoside linkages of said first oligonucleotide is at least 88%, preferably at least 90%, further preferably at least 92%.

Embodiment 191: The compound of any one of the preceding embodiments, wherein the percentage of internucleoside linkages which are unmodified phosphodiester and phosphorodiamidate internucleoside linkages relative to the total number of internucleoside linkages of said first oligonucleotide is at least 95%, preferably at least 100%.

Embodiment 192: The compound of any one of the preceding embodiments, wherein all internucleoside linkages of said first oligonucleotide are phosphorodiamidate internucleoside linkages.

Embodiment 193: The compound of any one of the preceding embodiments, wherein said modified internucleoside linkages are selected from the group consisting of phosphotriester, phosphonates, methylphosphonates, phosphoramidates, phosphorodiamidates, phosphorthioamidates, phosphinates, phosphorothioates (“P═S”), and phosphorodithioates.

Embodiment 194: The compound of any one of the preceding embodiments, wherein said modified internucleoside linkages are selected from the group consisting of methylphosphonates, phosphoramidates, phosphorodiamidates, phosphorothioates (“P═S”), and phosphorodithioates.

Embodiment 195: The compound of any one of the preceding embodiments, wherein the first oligonucleotide consists of 10-40 linked nucleosides.

Embodiment 196: The compound of any one of the preceding embodiments, wherein the first oligonucleotide consists of 12-30 linked nucleosides.

Embodiment 197: The compound of any one of the preceding embodiments, wherein the first oligonucleotide consists of 12-28 linked nucleosides.

Embodiment 198: The compound of any one of the preceding embodiments, wherein the first oligonucleotide consists of 12-25 linked nucleosides.

Embodiment 199: The compound of any one of the preceding embodiments, wherein the first oligonucleotide consists of 12-20 linked nucleosides.

Embodiment 200: The compound of any one of the preceding embodiments, wherein the Embodiment 184: The compound of any one of the preceding embodiments, wherein the second oligonucleotide consists of 12-30 linked nucleosides.

Embodiment 201: The compound of any one of the preceding embodiments, wherein the second oligonucleotide consists of 12-28 linked nucleosides.

Embodiment 202: The compound of any one of the preceding embodiments, wherein the second oligonucleotide consists of 12-25 linked nucleosides.

Embodiment 203: The compound of any one of the preceding embodiments, wherein the second oligonucleotide consists of 12-20 linked nucleosides.

Embodiment 204: The compound of any one of the preceding embodiments, wherein the first oligonucleotide and second oligonucleotide are equal in length, and thus comprise the same number of nucleosides and nucleotides.

Embodiment 205: The compound of any one of the preceding embodiments, wherein the second oligonucleotide 5, 6, 7, 8, 9, 10, 11, 12, 13, or 14 nucleobases shorter in length than the first oligonucleotide.

Embodiment 206: The compound of any one of the preceding embodiments, wherein the first oligonucleotide is at least 70% complementary to the nucleobase sequence of the target nucleic acid, when measured across the entire nucleobase sequence of the first modified oligonucleotide.

Embodiment 207: The compound of any one of the preceding embodiments, wherein the first oligonucleotide is at least 80% complementary to the nucleobase sequence of the target nucleic acid, when measured across the entire nucleobase sequence of the first modified oligonucleotide.

Embodiment 208: The compound of any one of the preceding embodiments, wherein the first oligonucleotide is at least 90% complementary to the nucleobase sequence of the target nucleic acid, when measured across the entire nucleobase sequence of the first modified oligonucleotide.

Embodiment 209: The compound of any one of the preceding embodiments, wherein the first oligonucleotide is 100% complementary to the nucleobase sequence of the target nucleic acid, when measured across the entire nucleobase sequence of the first modified oligonucleotide.

Embodiment 210: The compound of any one of the preceding embodiments, wherein the second oligonucleotide is at least 70% complementary to the nucleobase sequence of the first oligonucleotide, when measured across the entire nucleobase sequence of the second modified oligonucleotide.

Embodiment 211: The compound of any one of the preceding embodiments, wherein the second oligonucleotide is at least 80% complementary to the nucleobase sequence of the first oligonucleotide, when measured across the entire nucleobase sequence of the second modified oligonucleotide.

Embodiment 212: The compound of any one of the preceding embodiments, wherein the second oligonucleotide is at least 90% complementary to the nucleobase sequence of the first oligonucleotide, when measured across the entire nucleobase sequence of the second modified oligonucleotide.

Embodiment 213: The compound of any one of the preceding embodiments, wherein the second oligonucleotide is at least 70%, preferably at least 80% and further preferably at least 90% complementary to the nucleobase sequence of the first oligonucleotide, when measured across the entire nucleobase sequence of the second modified oligonucleotide, wherein each nucleoside of said first oligonucleotide is an abc-DNA nucleoside, and wherein the 3′-most nucleobase of the second modified oligonucleotide is complementary to the 5′-most nucleobase of the first modified oligonucleotide.

Embodiment 214: The compound of any one of the preceding embodiments, wherein the second oligonucleotide is at least 70%, preferably at least 80% and further preferably at least 90% complementary to the nucleobase sequence of the first oligonucleotide, when measured across the entire nucleobase sequence of the second modified oligonucleotide, wherein each nucleoside of said first oligonucleotide comprises a 2′-MOE sugar moiety, and wherein the 5′-most nucleobase of the second modified oligonucleotide is complementary to the 3′-most nucleobase of the first modified oligonucleotide.

Embodiment 215: The compound of any one of the preceding embodiments, wherein the second oligonucleotide is at least 70%, preferably at least 80% and further preferably at least 90% complementary to the nucleobase sequence of the first oligonucleotide, when measured across the entire nucleobase sequence of the second modified oligonucleotide, wherein each nucleoside of said first oligonucleotide is a 2′-MOE nucleobase, and wherein the 5′-most nucleobase of the second modified oligonucleotide is complementary to the 3′-most nucleobase of the first modified oligonucleotide.

Embodiment 216: The compound of any one of the preceding embodiments, wherein the second oligonucleotide is 100% complementary to the nucleobase sequence of the first oligonucleotide, when measured across the entire nucleobase sequence of the second modified oligonucleotide.

Embodiment 217 The compound of any one of the preceding embodiments, wherein the first oligonucleotide is at least 70% complementary to the nucleobase sequence of the second oligonucleotide, when measured across the entire nucleobase sequence of the first modified oligonucleotide.

Embodiment 218: The compound of any one of the preceding embodiments, wherein the first oligonucleotide is at least 80% complementary to the nucleobase sequence of the second oligonucleotide, when measured across the entire nucleobase sequence of the first modified oligonucleotide.

Embodiment 219: The compound of any one of the preceding embodiments, wherein the first oligonucleotide is at least 90% complementary to the nucleobase sequence of the second oligonucleotide, when measured across the entire nucleobase sequence of the first modified oligonucleotide.

Embodiment 220: The compound of any one of the preceding embodiments, wherein the first oligonucleotide is 100% complementary to the nucleobase sequence of the second oligonucleotide, when measured across the entire nucleobase sequence of the first modified oligonucleotide.

Embodiment 221: The compound of any one of the preceding embodiments, wherein the first oligonucleotide or the second modified oligonucleotide comprises at least one modified nucleobase.

Embodiment 222: The compound of any one of the preceding embodiments, wherein the first oligonucleotide or the second modified oligonucleotide comprises at least one modified nucleobase, wherein said modified nucleobase is a 5′-Me cytosine.

Embodiment 223: The compound of any one of the preceding embodiments, wherein each nucleobase of the first oligonucleotide and the second modified oligonucleotide is an unmodified nucleobase.

Embodiment 224: The compound of any one of the preceding embodiments, wherein each nucleobase of the first oligonucleotide and the second modified oligonucleotide is either an unmodified nucleobase or is 5′-Me cytosine.

Embodiment 225: The compound of any one of the preceding embodiments, wherein said first oligomeric compound does comprise a conjugate group.

Embodiment 226: The compound of any one of the preceding embodiments, wherein said first oligomeric compound does comprise a conjugate group, wherein the conjugate group is covalently attached to the 3′-end of the first oligonucleotide.

Embodiment 227: The compound of any one of the preceding embodiments, wherein said second oligomeric compound does comprise a conjugate group.

Embodiment 228: The compound of any one of the preceding embodiments, wherein said second oligomeric compound does comprise a conjugate group, wherein the conjugate group is covalently attached to the 3′-end of the first oligonucleotide.

Embodiment 229: The compound of any one of the preceding embodiments, wherein said second oligomeric compound does not comprise a conjugate group.

Embodiment 230: The compound of any one of the preceding embodiments, wherein said first oligomeric compound does not comprise a conjugate group.

Embodiment 231: The compound of any one of the preceding embodiments, wherein said first oligomeric compound does comprise a conjugate group, and wherein said conjugate group comprises a conjugate moiety, and wherein said conjugate moiety is a lipid group.

Embodiment 232: The compound of any one of the preceding embodiments, wherein said first oligomeric compound does comprise a conjugate group, and wherein said conjugate group comprises a conjugate moiety, and wherein said conjugate moiety is a lipid group, and wherein said wherein said conjugate group comprises a conjugate linker, and wherein said conjugate linker is a hydrocarbon linker or a polyethylene glycol (PEG) linker.

Embodiment 233: The compound of any one of the preceding embodiments, wherein said first oligomeric compound does comprise a conjugate group, and wherein said conjugate group comprises a conjugate moiety, and wherein said conjugate moiety is a lipid group, and wherein said wherein said conjugate group comprises a conjugate linker, and wherein said conjugate linker comprises a cleavable group.

Embodiment 234: The compound of any one of the preceding embodiments, wherein said first oligomeric compound does comprise a conjugate group, and wherein said conjugate group comprises a conjugate moiety, and wherein said conjugate moiety is a lipid group, and wherein said lipid group is a fatty acid, wherein preferably the fatty acid has a length from 4 to 28 carbon atoms.

Embodiment 235: The compound of any one of the preceding embodiments, wherein said first oligomeric compound does comprise a conjugate group, and wherein said conjugate group comprises a conjugate moiety, and wherein said conjugate moiety is a lipid group, and wherein said lipid group is a saturated or unsaturated fatty acid, wherein preferably the fatty acid has a length from 4 to 28 carbon atoms.

Embodiment 236: The compound of any one of the preceding embodiments, wherein said first oligomeric compound does comprise a conjugate group, and wherein said conjugate group comprises a conjugate moiety, and wherein said conjugate moiety is a lipid group, and wherein said lipid group is a saturated fatty acid, wherein preferably the fatty acid has a length from 4 to 28 carbon atoms.

Embodiment 237: The compound of any one of the preceding embodiments, wherein said first oligomeric compound does comprise a conjugate group, and wherein said conjugate group comprises a conjugate moiety, and wherein said conjugate moiety is a lipid group, and wherein said lipid group is an unsaturated fatty acid, wherein preferably the fatty acid has a length from 4 to 28 carbon atoms.

Embodiment 238: The compound of any one of the preceding embodiments, wherein said first oligomeric compound does comprise a conjugate group, and wherein said conjugate group comprises a conjugate moiety, and wherein said conjugate moiety is a lipid group, and wherein said lipid group is a fatty acid, wherein the fatty acid is hexadecanoic acid.

Embodiment 239: The compound of any one of the preceding embodiments, wherein said nucleic acid target is an extra-hepatic nucleic acid target.

Embodiment 240: The compound of any one of the preceding embodiments, wherein the affinity of said first oligonucleotide to said second oligonucleotide is lower than the affinity of said first oligonucleotide to the fully complementary unmodified RNA oligonucleotide of said first oligonucleotide; and wherein the biostability of said second oligonucleotide is lower than the biostability of said first oligonucleotide.

Embodiment 241: The compound of any one of the preceding embodiments, wherein said affinity of said first oligonucleotide to said second oligonucleotide is lower than the affinity of said first oligonucleotide to the fully complementary unmodified RNA oligonucleotide of said first oligonucleotide, and wherein said lower affinity corresponds to a lower melting temperature Tm of the duplex of said first and second oligonucleotide as compared to the melting temperature Tm of the duplex of said first oligonucleotide and the fully complementary unmodified RNA oligonucleotide of said first oligonucleotide.

Embodiment 242: The compound of any one of the preceding embodiments, wherein said affinity of said first oligonucleotide to said second oligonucleotide is lower than the affinity of said first oligonucleotide to the fully complementary unmodified RNA oligonucleotide of said first oligonucleotide, and wherein said lower affinity corresponds to a melting temperature Tm of the duplex of said first and second oligonucleotide which is at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40° C. lower than the melting temperature Tm of the duplex of said first oligonucleotide and the fully complementary unmodified RNA oligonucleotide of said first oligonucleotide.

Embodiment 243: The compound of any one of the preceding embodiments, wherein said affinity of said first oligonucleotide to said second oligonucleotide is lower than the affinity of said first oligonucleotide to the fully complementary unmodified RNA oligonucleotide of said first oligonucleotide, and wherein said lower affinity corresponds to a melting temperature Tm of the duplex of said first and second oligonucleotide which is at least 3° C., preferably 5° C., but at most 40° C., preferably 35° C., lower than the melting temperature Tm of the duplex of said first oligonucleotide and the fully complementary unmodified RNA oligonucleotide of said first oligonucleotide.

Embodiment 244: The compound of any one of the preceding embodiments, wherein said affinity of said first oligonucleotide to said second oligonucleotide is lower than the affinity of said first oligonucleotide to the fully complementary unmodified RNA oligonucleotide of said first oligonucleotide, and wherein said lower affinity corresponds to a melting temperature Tm of the duplex of said first and second oligonucleotide which is at least 8° C., preferably 10° C., but at most 40° C., preferably 35° C., lower than the melting temperature Tm of the duplex of said first oligonucleotide and the fully complementary unmodified RNA oligonucleotide of said first oligonucleotide.

Embodiment 245: The compound of any one of the preceding embodiments, wherein said affinity of said first oligonucleotide to said second oligonucleotide is lower than the affinity of said first oligonucleotide to the fully complementary unmodified RNA oligonucleotide of said first oligonucleotide, and wherein said lower affinity corresponds to a melting temperature Tm of the duplex of said first and second oligonucleotide which is at least 13° C., preferably 15° C., but at most 40° C., preferably 35° C., lower than the melting temperature Tm of the duplex of said first oligonucleotide and the fully complementary unmodified RNA oligonucleotide of said first oligonucleotide.

Embodiment 246: The compound of any one of the preceding embodiments, wherein said affinity of said first oligonucleotide to said second oligonucleotide is lower than the affinity of said first oligonucleotide to the fully complementary unmodified RNA oligonucleotide of said first oligonucleotide, and wherein said lower affinity corresponds to a melting temperature Tm of the duplex of said first and second oligonucleotide which is at least 18° C., preferably 20° C., but at most 40° C., preferably 35° C., lower than the melting temperature Tm of the duplex of said first oligonucleotide and the fully complementary unmodified RNA oligonucleotide of said first oligonucleotide.

Embodiment 247: The compound of any one of the preceding embodiments, wherein said affinity of said first oligonucleotide to said second oligonucleotide is lower than the affinity of said first oligonucleotide to the fully complementary unmodified RNA oligonucleotide of said first oligonucleotide, and wherein said lower affinity corresponds to a melting temperature Tm of the duplex of said first and second oligonucleotide which is at least 23° C., preferably 25° C., but at most 40° C., preferably 35° C., lower than the melting temperature Tm of the duplex of said first oligonucleotide and the fully complementary unmodified RNA oligonucleotide of said first oligonucleotide.

Embodiment 248: The compound of any one of the preceding embodiments, wherein said affinity of said first oligonucleotide to said second oligonucleotide is lower than the affinity of said first oligonucleotide to the fully complementary unmodified RNA oligonucleotide of said first oligonucleotide, and wherein said lower affinity corresponds to a melting temperature Tm of the duplex of said first and second oligonucleotide which is at least 25° C. but at most 40° C., preferably 35° C., lower than the melting temperature Tm of the duplex of said first oligonucleotide and the fully complementary unmodified RNA oligonucleotide of said first oligonucleotide.

Embodiment 249: The compound of any one of the preceding embodiments, wherein said melting temperatures Tm are determined as described in Example 2.

Embodiment 250: The compound of any one of the preceding embodiments, wherein the biostability of said second oligonucleotide is lower than the biostability of said first oligonucleotide, wherein said lower biostability corresponds to a lower half-life stability in serum.

Embodiment 251: The compound of any one of the preceding embodiments, wherein the biostability of said second oligonucleotide is lower than the biostability of said first oligonucleotide, wherein said lower biostability corresponds to a lower half-life stability in mouse serum.

Embodiment 252: The compound of any one of the preceding embodiments, wherein the biostability of said second oligonucleotide is lower than the biostability of said first oligonucleotide, wherein said lower biostability corresponds to a lower half-life stability in human serum.

Embodiment 253: The compound of any one of the preceding embodiments, wherein the biostability of said second oligonucleotide is lower than the biostability of said first oligonucleotide, wherein said lower biostability corresponds to a lower half-life stability in serum, preferably mice or human serum, as determined by AEX-chromatography.

Embodiment 254: The compound of any one of the preceding embodiments, wherein the biostability of said second oligonucleotide is lower than the biostability of said first oligonucleotide, wherein said lower biostability corresponds to a lower half-life stability in serum, preferably mice or human serum, as determined by AEX-chromatography after denaturating the duplex of said first and said second oligonucleotide.

Embodiment 255: The compound of any one of the preceding embodiments, wherein the half-life stability of said second oligonucleotide is at least 30, 45, 60 minutes and at most 100 hours, preferably at most 72 hours.

Embodiment 256: The compound of any one of the preceding embodiments, wherein the half-life stability of said second oligonucleotide is at least 1 hour, preferably at least 2 hours, and at most 100 hours, preferably at most 72 hours.

Embodiment 257: The compound of any one of the preceding embodiments, wherein the biostability corresponds to a lower half-life stability in serum, preferably in mouse or human serum, further preferably in mouse serum as determined by AEX-chromatography after denaturating the duplex of said first and said second oligonucleotide as described in Example 3.

Embodiment 258: The compound of any one of the preceding embodiments, wherein the melting temperature Tm of the duplex of said first oligonucleotide and said second oligonucleotide is at least 40° C., preferably at least 45° C., typically and preferably wherein said melting temperature Tm is determined as described in Example 2.

Embodiment 259: The compound of any of the preceding embodiments, wherein at least 10% of the nucleosides in the first oligonucleotide are abc-DNA nucleosides.

Embodiment 260: The compound of any of the preceding embodiments, wherein at least 20% of the nucleosides in the first oligonucleotide are abc-DNA nucleosides.

Embodiment 261: The compound of any of the preceding embodiments, wherein at least 30% of the nucleosides in the first oligonucleotide are abc-DNA nucleosides.

Embodiment 262: The compound of any of the preceding embodiments, wherein at least 40% of the nucleosides in the first oligonucleotide are abc-DNA nucleosides.

Embodiment 263: The compound of any of the preceding embodiments, wherein at least 50% of the nucleosides in the first oligonucleotide are abc-DNA nucleosides.

Embodiment 264: The compound of any of the preceding embodiments, wherein at least 60% of the nucleosides in the first oligonucleotide are abc-DNA nucleosides.

Embodiment 265: The compound of any of the preceding embodiments, wherein at least 70% of the nucleosides in the first oligonucleotide are abc-DNA nucleosides.

Embodiment 266: The compound of any of the preceding embodiments, wherein at least 80% of the nucleosides in the first oligonucleotide are abc-DNA nucleosides.

Embodiment 267: The compound of any of the preceding embodiments, wherein at least 90% of the nucleosides in the first oligonucleotide are abc-DNA nucleosides.

Embodiment 268: The compound of any of the preceding embodiments, wherein at least 95% of the nucleosides in the first oligonucleotide are abc-DNA nucleosides.

Embodiment 269: The compound of any of the preceding embodiments, wherein 100% of the nucleosides in the first oligonucleotide are abc-DNA nucleosides.

Embodiment 270: The compound of any one of the preceding embodiments, wherein the first oligonucleotide is a gapmer comprising at least two abc-DNA nucleosides, wherein at least one non-abc-DNA nucleoside is present between said two abc-DNA nucleosides.

Embodiment 271: The compound of any one of the preceding embodiments, wherein the first oligonucleotide is a gapmer comprising at least two abc-DNA nucleosides, wherein at least one unmodified DNA nucleoside is present between said two abc-DNA nucleosides.

Embodiment 272: The compound of any of the preceding embodiments, wherein the first oligonucleotide is a gapmer wherein the 5′ wing of the gapmer comprises 1-10 abc-DNA nucleosides.

Embodiment 273: The compound of any of the preceding embodiments, wherein the first oligonucleotide is a gapmer wherein the 5′ wing of the gapmer comprises 1-6 abc-DNA nucleosides.

Embodiment 274: The compound of any of the preceding embodiments, wherein the first oligonucleotide is a gapmer wherein the 5′ wing of the gapmer comprises 1-5 abc-DNA nucleosides.

Embodiment 275: The compound of any of the preceding embodiments, wherein the first oligonucleotide is a gapmer wherein the 5′ wing of the gapmer comprises 1-4 abc-DNA nucleosides.

Embodiment 276: The compound of any of the preceding embodiments, wherein the first oligonucleotide is a gapmer wherein the 5′ wing of the gapmer comprises 1-3 abc-DNA nucleosides.

Embodiment 277: The compound of any of the preceding embodiments, wherein the first oligonucleotide is a gapmer wherein the 5′ wing of the gapmer comprises 1-2 abc-DNA nucleosides.

Embodiment 278: The compound of any of the preceding embodiments, wherein the first oligonucleotide is a gapmer wherein the 5′ wing of the gapmer comprises 2-5 abc-DNA nucleosides.

Embodiment 279: The compound of any of the preceding embodiments, wherein the first oligonucleotide is a gapmer wherein the 5′ wing of the gapmer comprises 2-4 abc-DNA nucleosides.

Embodiment 280: The compound of any of the preceding embodiments, wherein the first oligonucleotide is a gapmer wherein the 3′ wing of the gapmer comprises 1-10 abc-DNA nucleosides.

Embodiment 281: The compound of any of the preceding embodiments, wherein the first oligonucleotide is a gapmer wherein the 3′ wing of the gapmer comprises 1-6 abc-DNA nucleosides.

Embodiment 282: The compound of any of the preceding embodiments, wherein the first oligonucleotide is a gapmer wherein the 3′ wing of the gapmer comprises 1-5 abc-DNA nucleosides.

Embodiment 283: The compound of any of the preceding embodiments, wherein the first oligonucleotide is a gapmer wherein the 3′ wing of the gapmer comprises 1-4 abc-DNA nucleosides.

Embodiment 284: The compound of any of the preceding embodiments, wherein first oligonucleotide is a gapmer wherein the 3′ wing of the gapmer comprises 1-3 abc-DNA nucleosides.

Embodiment 285: The compound of any of the preceding embodiments, wherein the first oligonucleotide is a gapmer wherein the 3′ wing of the gapmer comprises 1-2 abc-DNA nucleosides.

Embodiment 286: The compound of any of the preceding embodiments, wherein the first oligonucleotide is a gapmer wherein the 3′ wing of the gapmer comprises 2-5 abc-DNA nucleosides.

Embodiment 287: The compound of any of the preceding embodiments, wherein the first oligonucleotide is a gapmer wherein the 3′ wing of the gapmer comprises 2-4 abc-DNA nucleosides.

Embodiment 288: The compound of any of the preceding embodiments, wherein the first oligonucleotide is a gapmer wherein the 5′ wing and the 3′ wing of the gapmer both comprise 1-5 abc-DNA nucleosides.

Embodiment 289: The compound of any of the preceding embodiments, wherein the first oligonucleotide is a gapmer wherein the 5′ wing and the 3′ wing of the gapmer both comprise 1-4 abc-DNA nucleosides.

Embodiment 290: The compound of any of the preceding embodiments, wherein the first oligonucleotide is a gapmer wherein the 5′ wing and the 3′ wing of the gapmer both comprise 1-3 abc-DNA nucleosides.

Embodiment 291: The compound of any of the preceding embodiments, wherein the first oligonucleotide is a gapmer wherein the 5′ wing and the 3′ wing of the gapmer both comprise 1-2 abc-DNA nucleosides.

Embodiment 292: The compound of any of the preceding embodiments, wherein the first oligonucleotide is a gapmer wherein the 5′ wing and the 3′ wing of the gapmer both comprise 2-5 abc-DNA nucleosides.

Embodiment 293: The compound of any of the preceding embodiments, wherein the first oligonucleotide is a gapmer wherein the 5′ wing and the 3′ wing of the gapmer both comprise 2-4 abc-DNA nucleosides.

Embodiment 294: The compound of any of the preceding embodiments, wherein the first oligonucleotide is a gapmer wherein the gap of said gapmer comprises 7-12 unmodified DNA nucleosides.

Embodiment 295: The compound of any of the preceding embodiments, wherein the first oligonucleotide is a gapmer and wherein at least 10% of the nucleosides in the first oligonucleotide are abc-DNA nucleosides.

Embodiment 296: The compound of any of the preceding embodiments, wherein the first oligonucleotide is a gapmer and wherein at least 20% of the nucleosides in the first oligonucleotide are abc-DNA nucleosides.

Embodiment 297: The compound of any of the preceding embodiments, wherein the first oligonucleotide is a gapmer and wherein at least 25% of the nucleosides in the first oligonucleotide are abc-DNA nucleosides.

Embodiment 298: The compound of any of the preceding embodiments, wherein the first oligonucleotide is a gapmer and wherein at least 30% of the nucleosides in the first oligonucleotide are abc-DNA nucleosides.

Embodiment 299: The compound of any of the preceding embodiments, wherein the first oligonucleotide is a gapmer and wherein at least 35% of the nucleosides in the first oligonucleotide are abc-DNA nucleosides.

Embodiment 300: The compound of any of the preceding embodiments, wherein the first oligonucleotide is a gapmer and wherein at least 40% of the nucleosides in the first oligonucleotide are abc-DNA nucleosides.

Embodiment 301: The compound of any of the preceding embodiments, wherein the first oligonucleotide is a gapmer and wherein at least 45% of the nucleosides in the first oligonucleotide are abc-DNA nucleosides.

Embodiment 302: The compound of any of the preceding embodiments, wherein the first oligonucleotide is a gapmer and wherein at least 50% of the nucleosides in the first oligonucleotide are abc-DNA nucleosides.

Embodiment 303: The compound of any of the preceding embodiments, wherein the first oligonucleotide is a gapmer wherein the gap of said gapmer comprises at least one unmodified DNA nucleoside.

Embodiment 304: The compound of any of the preceding embodiments, wherein the first oligonucleotide is a gapmer wherein the gap of said gapmer comprises at least 2 unmodified DNA nucleosides, preferably at least 3 unmodified DNA nucleosides, more preferably at least 4 unmodified DNA nucleosides, yet more preferably at least 5 unmodified DNA nucleosides.

Embodiment 305: The compound of any of the preceding embodiments, wherein the first oligonucleotide is a gapmer wherein at least 50% of the nucleosides in the gap of said gapmer are unmodified DNA nucleosides; preferably at least 60%, more preferably at least 70%, yet more preferably at least 80%, yet more preferably at least 90% of the nucleosides in the gap are unmodified DNA nucleosides.

Embodiment 306: The compound of any of the preceding embodiments, wherein the first oligonucleotide is a gapmer wherein each nucleoside of the gap of said gapmer is an unmodified DNA nucleoside.

Embodiment 307: The compound of any of the preceding embodiments, wherein the first oligonucleotide is a gapmer wherein each nucleoside of the gap of said gapmer is an unmodified DNA nucleoside, and wherein at most three of all internucleoside linkages of said first oligonucleotide are modified internucleoside linkages.

Embodiment 308: The compound of any of the preceding embodiments, wherein the first oligonucleotide is a gapmer, wherein the gap of said gapmer comprises at least one modified nucleoside, wherein said modified nucleoside comprises a modified sugar moiety selected from the group consisting of (i) a non-bicyclic modified sugar moiety comprising a furanosyl ring with one or more acyclic substituents at the 2′, 4′, and/or 5′ position, (ii) a bicyclic sugar moiety, (iii) a tricyclic sugar moiety and (iv) a sugar surrogate.

Embodiment 309: The compound of any of the preceding embodiments, wherein the first oligonucleotide is a gapmer, wherein the gap of said gapmer comprises at least one modified nucleoside, wherein said modified nucleoside is selected from the group consisting of a 2′-MOE nucleoside, 2′-OMe nucleoside, a LNA, a cET, cMOE, a CeNA, a HNA, a ANA, a FANA, a TNA, a tcDNA, a PNA, a GNA, a morpholino or a modified morpholino.

Embodiment 310: The compound of any of the preceding embodiments, wherein said first oligonucleotide is a gapmer, wherein the gap of said gapmer of said first oligonucleotide has an antisense effect on said nucleic acid target.

Embodiment 311: The compound of any of the preceding embodiments, wherein said first oligonucleotide is a gapmer, wherein the gap of said gapmer of said first oligonucleotide comprises at least one modified nucleoside, wherein said modified nucleoside comprises a non-bicyclic modified sugar moiety comprising a furanosyl ring with an acyclic substituent at the 2′ position. In a preferred embodiment, said acyclic substituent at the 2′ position is selected from 2′-F, 2′-OH, 2′-propargyl, 2′-O-propylamino, 2′-NH₂, 2′-OCH₃ (“OMe” or “O-methyl”), and 2′-O(CH₂)₂OCH₃ (“MOE”).

Embodiment 312: The compound of any of the preceding embodiments, wherein said first oligonucleotide is a gapmer, wherein the gap of said gapmer of said first oligonucleotide comprises at least one modified nucleoside, and wherein said modified nucleoside comprises a non-bicyclic modified sugar moiety comprising a furanosyl ring with an acyclic substituent at the 2′ position, wherein said acyclic substituent at the 2′ position is 2′-O(CH₂)₂OCH₃ (“MOE”).

Embodiment 313: The compound of any of the preceding embodiments, wherein said first oligonucleotide is a gapmer, wherein the gap of said gapmer of said first oligonucleotide comprises at least one modified nucleoside, wherein said modified nucleoside comprises a bicyclic sugar moiety, wherein said bicyclic sugar moiety comprises a bridge between the 4′ and the 3′ furanose ring atoms or a bridge between the 4′ and the 2′ furanose ring atoms.

Embodiment 314: The compound of any of the preceding embodiments, wherein said first oligonucleotide is a gapmer, wherein the gap of said gapmer of said first oligonucleotide comprises at least one modified nucleoside, wherein said modified nucleoside comprises a tricyclic sugar moiety.

Embodiment 315: The compound of any of the preceding embodiments, wherein said first oligonucleotide is a gapmer, wherein the gap of said gapmer of said first oligonucleotide comprises at least one modified nucleoside, wherein said modified nucleoside comprises a sugar surrogate.

Embodiment 316: The compound of any of the preceding embodiments, wherein said first oligonucleotide is a gapmer, wherein the gap of said gapmer of said first oligonucleotide comprises at least one modified nucleoside, wherein said modified nucleoside comprises a sugar surrogate, and wherein said sugar surrogate is a morpholino.

Embodiment 317: The compound of any of the preceding embodiments, wherein said first oligonucleotide is a gapmer, wherein each nucleoside of the gap of said gapmer of said first oligonucleotide is a modified nucleoside, wherein said modified nucleoside comprises a modified sugar moiety selected from the group consisting of (i) a non-bicyclic modified sugar moiety comprising a furanosyl ring with one or more acyclic substituent at the 2′, 4′, and/or 5′ position, (ii) a bicyclic sugar moiety, (iii) a tricyclic sugar moiety and (iv) a sugar surrogate.

Embodiment 318: The compound of any of the preceding embodiments, wherein said first oligonucleotide is a gapmer, wherein each nucleoside of the gap of said gapmer of said first oligonucleotide is a modified nucleoside, wherein said modified nucleoside comprises a non-bicyclic modified sugar moiety comprising a furanosyl ring with an acyclic substituent at the 2′ position. In a preferred embodiment, said acyclic substituent at the 2′ position is selected from 2′-F, 2′-OH, 2′-propargyl, 2′-O-propylamino, 2′-NH₂, 2′-OCH₃ (“OMe” or “O-methyl”), and 2′-O(CH₂)₂OCH₃ (“MOE”).

Embodiment 319: The compound of any of the preceding embodiments, wherein said first oligonucleotide is a gapmer, wherein each nucleoside of the gap of said gapmer of said first oligonucleotide is a non-bicyclic modified sugar moiety comprising a furanosyl ring with an acyclic substituent at the 2′ position, wherein said acyclic substituent at the 2′ position is 2′-O(CH₂)₂OCH₃ (“MOE”).

Embodiment 320: The compound of any of the preceding embodiments, wherein said first oligonucleotide is a gapmer, wherein each nucleoside of the gap of said gapmer of said first oligonucleotide is a bicyclic sugar moiety, wherein said bicyclic sugar moiety comprises a bridge between the 4′ and the 3′ furanose ring atoms or a bridge between the 4′ and the 2′ furanose ring atoms.

Embodiment 321: The compound of any of the preceding embodiments, wherein said first oligonucleotide is a gapmer, wherein each nucleoside of the gap of said gapmer of said first oligonucleotide is a tricyclic sugar moiety.

Embodiment 322: The compound of any of the preceding embodiments, wherein said first oligonucleotide is a gapmer, wherein each nucleoside of the gap of said gapmer of said first oligonucleotide is a sugar surrogate.

Embodiment 323: The compound of any of the preceding embodiments, wherein said first oligonucleotide is a gapmer, wherein each nucleoside of the gap of said gapmer of said first oligonucleotide is a sugar surrogate, and wherein said sugar surrogate is a morpholino.

Embodiment 324: The compound of any of the preceding embodiments, wherein said first oligonucleotide is a gapmer, wherein the gap of said gapmer of said first oligonucleotide comprises at least one modified nucleoside, wherein said modified nucleoside is selected from the group consisting of a 2′-MOE nucleoside and a morpholino.

Embodiment 325: The compound of any of the preceding embodiments, wherein said first oligonucleotide is a gapmer, wherein the gap of said gapmer of said first oligonucleotide comprises at least one modified nucleoside, wherein said modified nucleoside comprises a 2′-MOE sugar moiety.

Embodiment 326: The compound of any of the preceding embodiments, wherein said first oligonucleotide is a gapmer, wherein the gap of said gapmer of said first oligonucleotide comprises at least one modified nucleoside, wherein said modified nucleoside is a morpholino.

Embodiment 327: The compound of any of the preceding embodiments, wherein said first oligonucleotide is a gapmer, wherein each nucleoside of the gap of said gapmer of said first oligonucleotide is selected from the group consisting of a 2′-MOE nucleoside, 2′-OMe nucleoside, an abc-DNA nucleoside, a LNA, a cET, cMOE, a CeNA, a HNA, a ANA, a FANA, a TNA, a tcDNA, a PNA, a GNA, a morpholino or a modified morpholino.

Embodiment 328: The compound of any of the preceding embodiments, wherein said first oligonucleotide is a gapmer, wherein each nucleoside of the gap of said gapmer of said first oligonucleotide is selected from the group consisting of a 2′-MOE nucleoside, an abc-DNA nucleoside and a morpholino.

Embodiment 329: The compound of any of the preceding embodiments, wherein said first oligonucleotide is a gapmer, wherein each nucleoside of the gap of said gapmer of said first oligonucleotide comprises a 2′-MOE sugar moiety.

Embodiment 330: The compound of any of the preceding embodiments, wherein said first oligonucleotide is a gapmer, wherein each nucleoside of the gap of said gapmer of said first oligonucleotide is a 2′-MOE nucleoside.

Embodiment 331: The compound of any of the preceding embodiments, wherein said first oligonucleotide is a gapmer, wherein each nucleoside of the gap of said gapmer of said first oligonucleotide is a morpholino.

Embodiment 332: The compound of any of the preceding embodiments, wherein said first oligonucleotide is a gapmer, wherein the gap of said gapmer of said first oligonucleotide is a modified oligonucleotide, and wherein said modified first oligonucleotide is a uniformly modified oligonucleotide.

Embodiment 333: The compound of any of the preceding embodiments, wherein said first oligonucleotide is a gapmer, wherein the gap of said gapmer of said first oligonucleotide is a modified oligonucleotide, and wherein each nucleobase of said modified first oligonucleotide is an unmodified nucleobase.

Embodiment 334: The compound of any of the preceding embodiments, wherein said first oligonucleotide is a gapmer, and wherein each sugar moiety of said second oligonucleotide is an unmodified DNA sugar moiety or unmodified RNA sugar moiety.

Embodiment 335: The compound of any of the preceding embodiments, wherein said first oligonucleotide is a gapmer, and wherein each sugar moiety of said second oligonucleotide is an unmodified DNA sugar moiety.

Embodiment 336: The compound of any of the preceding embodiments, wherein said first oligonucleotide is a gapmer, and wherein each sugar moiety of said second oligonucleotide is an unmodified RNA sugar moiety.

Embodiment 337: The compound of any one of the preceding embodiments, wherein said first oligonucleotide is a gapmer, and wherein each sugar moiety of said second oligonucleotide is an unmodified RNA sugar moiety, wherein at most five, preferably at most four of all internucleoside linkages of said second oligonucleotide are phosphorothioate internucleoside linkages, wherein preferably said phosphorothioate internucleoside linkages are positioned at the 5′-end and/or at the 3′-end of said second oligonucleotide.

Embodiment 338: The compound of any one of the preceding embodiments, wherein said first oligonucleotide is a gapmer, and wherein each sugar moiety of said second oligonucleotide is an unmodified RNA sugar moiety, wherein at most five, preferably at most four of all internucleoside linkages of said second oligonucleotide are modified internucleoside linkages, wherein preferably said modified internucleoside linkages are positioned at the 5′-end and/or at the 3′-end of said second oligonucleotide.

Embodiment 339: The compound of any one of the preceding embodiments, wherein said first oligonucleotide is a gapmer, and wherein each sugar moiety of said second oligonucleotide is an unmodified RNA sugar moiety, wherein at most three of all internucleoside linkages of said second oligonucleotide are phosphorothioate internucleoside linkages, wherein preferably said phosphorothioate internucleoside linkages are positioned at the 5′-end and/or at the 3′-end of said second oligonucleotide.

Embodiment 340: The compound of any one of the preceding embodiments, wherein said first oligonucleotide is a gapmer, and wherein each sugar moiety of said second oligonucleotide is an unmodified RNA sugar moiety, wherein at most three of all internucleoside linkages of said second oligonucleotide are modified internucleoside linkages, wherein preferably said modified internucleoside linkages are positioned at the 5′-end and/or at the 3′-end of said second oligonucleotide.

Embodiment 341: The compound of any one of the preceding embodiments, wherein said first oligonucleotide is a gapmer, and wherein each sugar moiety of said second oligonucleotide is an unmodified RNA sugar moiety, wherein at most two of all internucleoside linkages of said second oligonucleotide are phosphorothioate internucleoside linkages, and wherein preferably said one or two phosphorothioate internucleoside linkages are at the 3′end of said second oligonucleotide.

Embodiment 342: The compound of any one of the preceding embodiments, wherein said first oligonucleotide is a gapmer, and wherein each sugar moiety of said second oligonucleotide is an unmodified RNA sugar moiety, wherein at most two of all internucleoside linkages of said second oligonucleotide are modified internucleoside linkages, and wherein preferably said one or two modified internucleoside linkages are at the 3′end of said second oligonucleotide.

Embodiment 343: The compound of any one of the preceding embodiments, wherein said first oligonucleotide is a gapmer, and wherein each sugar moiety of said second oligonucleotide is an unmodified RNA sugar moiety, wherein at most one of all internucleoside linkages of said second oligonucleotide is a phosphorothioate internucleoside linkage, and wherein preferably said one phosphorothioate internucleoside linkages is at the 3′end of said second oligonucleotide.

Embodiment 344: The compound of any one of the preceding embodiments, wherein said first oligonucleotide is a gapmer, and wherein each sugar moiety of said second oligonucleotide is an unmodified RNA sugar moiety, wherein at most one of all internucleoside linkages of said second oligonucleotide is a modified internucleoside linkage, and wherein preferably said one modified internucleoside linkage is at the 3′end of said second oligonucleotide.

Embodiment 345: The compound of any one of the preceding embodiments, wherein said first oligonucleotide is a gapmer, and wherein each nucleobase of said second oligonucleotide is an unmodified nucleobase.

Embodiment 346: The compound of any one of the preceding embodiments, wherein said first oligonucleotide is a gapmer, and wherein said second oligonucleotide comprises at least one unmodified nucleoside and at least one modified nucleoside, wherein said at least one unmodified nucleoside is an unmodified RNA nucleoside, and wherein said modified nucleoside comprises a modified sugar moiety selected from the group consisting of (i) a non-bicyclic modified sugar moiety comprising a furanosyl ring with one or more acyclic substituent at the 2′, 4′, and/or 5′ position, (ii) a bicyclic sugar moiety, (iii) a tricyclic sugar moiety and (iv) a sugar surrogate.

Embodiment 347: The compound of any one of the preceding embodiments, wherein said first oligonucleotide is a gapmer, and wherein said second oligonucleotide comprises at least one unmodified nucleoside and at least one modified nucleoside, wherein said at least one unmodified nucleoside is an unmodified RNA nucleoside, and wherein said at least one modified nucleoside is selected from the group consisting of a 2′-MOE nucleoside, 2′-OMe nucleoside, an abc-DNA nucleoside, a LNA, a cET, cMOE, a CeNA, a HNA, a ANA, a FANA, a TNA, a tcDNA, a PNA, a GNA, a morpholino or a modified morpholino.

Embodiment 348: The compound of any one of the preceding embodiments, wherein said first oligonucleotide is a gapmer, and wherein said second oligonucleotide comprises at least one unmodified nucleoside and at least one modified nucleoside, wherein said at least one unmodified nucleoside is an unmodified RNA nucleoside, and wherein said modified nucleoside comprises a non-bicyclic modified sugar moiety comprising a furanosyl ring with an acyclic substituent at the 2′ position.

Embodiment 349: The compound of any one of the preceding embodiments, wherein said acyclic substituent at the 2′ position is selected from 2′-F, 2′-OH, 2′-propargyl, 2′-O-propylamino, 2′-NH₂, 2′-OCH₃ (“OMe” or “O-methyl”), and 2′-O(CH₂)₂OCH₃ (“MOE”).

Embodiment 350: The compound of any one of the preceding embodiments, wherein said first oligonucleotide is a gapmer, and wherein said second oligonucleotide comprises at least one unmodified nucleoside and at least one modified nucleoside, wherein said at least one unmodified nucleoside is an unmodified RNA nucleoside, and wherein said modified nucleoside comprises a non-bicyclic modified sugar moiety comprising a furanosyl ring with an acyclic substituent at the 2′ position, wherein said acyclic substituent at the 2′ position is 2′-O(CH₂)₂OCH₃ (“MOE”).

Embodiment 351: The compound of any one of the preceding embodiments, wherein said first oligonucleotide is a gapmer, and wherein said second oligonucleotide comprises at least one unmodified nucleoside and at least one modified nucleoside, wherein said at least one unmodified nucleoside is an unmodified RNA nucleoside, and wherein said modified nucleoside comprises a non-bicyclic modified sugar moiety comprising a furanosyl ring with an acyclic substituent at the 2′ position, wherein said acyclic substituent at the 2′ position is 2′-OCH₃ (“OMe” or “O-methyl”).

Embodiment 352: The compound of any one of the preceding embodiments, wherein said first oligonucleotide is a gapmer, and wherein said second oligonucleotide comprises at least one unmodified nucleoside and at least one modified nucleoside, wherein said at least one unmodified nucleoside is an unmodified RNA nucleoside, and wherein said modified nucleoside a bicyclic sugar moiety, wherein said bicyclic sugar moiety comprises a bridge between the 4′ and the 3′ furanose ring atoms or a bridge between the 4′ and the 2′ furanose ring atoms.

Embodiment 353: The compound of any one of the preceding embodiments, wherein said first oligonucleotide is a gapmer, and wherein said second oligonucleotide comprises at least one unmodified nucleoside and at least one modified nucleoside, wherein said at least one unmodified nucleoside is an unmodified RNA nucleoside, and wherein said modified nucleoside is selected from the group consisting of a 2′-MOE nucleoside, 2′-OMe nucleoside, an abc-RNA nucleoside, a LNA, a cET, cMOE, a CeNA, a HNA, a ANA, a FANA, a TNA, a tcDNA, a PNA, a GNA, a morpholino or a modified morpholino.

Embodiment 354: The compound of any one of the preceding embodiments, wherein said first oligonucleotide is a gapmer, and wherein said second oligonucleotide comprises at least one unmodified nucleoside and at least one modified nucleoside, wherein said at least one unmodified nucleoside is an unmodified RNA nucleoside, and wherein said modified nucleoside a tricyclic sugar moiety.

Embodiment 355: The compound of any one of the preceding embodiments, wherein said first oligonucleotide is a gapmer, and wherein said second oligonucleotide comprises at least one unmodified nucleoside and at least one modified nucleoside, wherein said at least one unmodified nucleoside is an unmodified RNA nucleoside, and wherein said modified nucleoside is a sugar surrogate.

Embodiment 356: The compound of any one of the preceding embodiments, wherein said first oligonucleotide is a gapmer, and wherein said second oligonucleotide comprises at least one unmodified nucleoside and at least one modified nucleoside, wherein said at least one unmodified nucleoside is an unmodified RNA nucleoside, and wherein said modified nucleoside is a sugar surrogate, and wherein said sugar surrogate is a morpholino.

Embodiment 357: The compound of any one of the preceding embodiments, wherein said first oligonucleotide is a gapmer, and wherein said second oligonucleotide is a modified second oligonucleotide comprising unmodified nucleosides and at least one but at most four modified nucleosides, wherein said unmodified nucleosides are unmodified RNA nucleosides, and wherein said modified nucleoside comprises a modified sugar moiety selected from the group consisting of (i) a non-bicyclic modified sugar moiety comprising a furanosyl ring with one or more acyclic substituent at the 2′, 4′, and/or 5′ position, (ii) a bicyclic sugar moiety, (iii) a tricyclic sugar moiety and (iv) a sugar surrogate.

Embodiment 358: The compound of any one of the preceding embodiments, wherein said first oligonucleotide is a gapmer, and wherein said second oligonucleotide is a modified second oligonucleotide comprising unmodified nucleosides and at least one but at most four modified nucleosides, wherein said unmodified nucleosides are unmodified RNA nucleosides, and wherein said modified nucleoside comprises a non-bicyclic modified sugar moiety comprising a furanosyl ring with an acyclic substituent at the 2′ position. In a preferred embodiment, said acyclic substituent at the 2′ position is selected from 2′-F, 2′-OH, 2′-propargyl, 2′-O-propylamino, 2′-NH₂, 2′-OCH₃ (“OMe” or “O-methyl”), and 2′-O(CH₂)₂OCH₃ (“MOE”).

Embodiment 359: The compound of any one of the preceding embodiments, wherein said first oligonucleotide is a gapmer, and wherein said second oligonucleotide is a modified second oligonucleotide comprising unmodified nucleosides and at least one but at most four modified nucleosides, wherein said unmodified nucleosides are unmodified RNA nucleosides, and wherein said modified nucleoside comprises a non-bicyclic modified sugar moiety comprising a furanosyl ring with an acyclic substituent at the 2′ position, wherein said acyclic substituent at the 2′ position is 2′-O(CH₂)₂OCH₃ (“MOE”).

Embodiment 360: The compound of any one of the preceding embodiments, wherein said first oligonucleotide is a gapmer, and wherein said second oligonucleotide is a modified second oligonucleotide comprising unmodified nucleosides and at least one but at most four modified nucleosides, wherein said unmodified nucleosides are unmodified RNA nucleosides, and wherein said modified nucleoside comprises a non-bicyclic modified sugar moiety comprising a furanosyl ring with an acyclic substituent at the 2′ position, wherein said acyclic substituent at the 2′ position is 2′-OCH₃ (“OMe” or “O-methyl”).

Embodiment 361: The compound of any one of the preceding embodiments, wherein said first oligonucleotide is a gapmer, and wherein said second oligonucleotide is a modified second oligonucleotide comprising unmodified nucleosides and at least one but at most four modified nucleosides, wherein said unmodified nucleosides are unmodified RNA nucleosides, and wherein said modified nucleoside a bicyclic sugar moiety, wherein said bicyclic sugar moiety comprises a bridge between the 4′ and the 3′ furanose ring atoms or a bridge between the 4′ and the 2′ furanose ring atoms.

Embodiment 362: The compound of any one of the preceding embodiments, wherein said first oligonucleotide is a gapmer, and wherein said second oligonucleotide is a modified second oligonucleotide comprising unmodified nucleosides and at least one but at most four modified nucleosides, wherein said unmodified nucleosides are unmodified RNA nucleosides, and wherein said modified nucleoside is selected from the group consisting of a 2′-MOE nucleoside, 2′-OMe nucleoside, an abc-DNA nucleoside, a LNA, a cET, cMOE, a CeNA, a HNA, a ANA, a FANA, a TNA, a tcDNA, a PNA, a GNA, a morpholino or a modified morpholino.

Embodiment 363: The compound of any one of the preceding embodiments, wherein said first oligonucleotide is a gapmer, and wherein said second oligonucleotide is a modified second oligonucleotide comprising unmodified nucleosides and at least one but at most three modified nucleosides, wherein said unmodified nucleosides are unmodified RNA nucleosides, and wherein said modified nucleoside is selected from the group consisting of a 2′-MOE nucleoside, 2′-OMe nucleoside, an abc-DNA nucleoside, a LNA, a cET, a HNA, a ANA, a FANA, a tcDNA, a PNA, a morpholino or a modified morpholino.

Embodiment 364: The compound of any one of the preceding embodiments, wherein said first oligonucleotide is a gapmer, and wherein said second oligonucleotide is a modified second oligonucleotide comprising unmodified nucleosides and at least one but at most two modified nucleosides, wherein said unmodified nucleosides are unmodified RNA nucleosides, and wherein said modified nucleoside is selected from the group consisting of a 2′-MOE nucleoside, 2′-OMe nucleoside, an abc-DNA nucleoside, a LNA, a cET, a HNA, a ANA, a FANA, a tcDNA and a morpholino.

Embodiment 365: The compound of any one of the preceding embodiments, wherein said first oligonucleotide is a gapmer, and wherein said second oligonucleotide is a modified second oligonucleotide comprising unmodified nucleosides and at least one but at most one modified nucleosides, wherein said unmodified nucleosides are unmodified RNA nucleosides, and wherein said modified nucleoside is selected from the group consisting of a 2′-MOE nucleoside, 2′-OMe nucleoside, an abc-DNA nucleoside, a LNA, a cET, a ANA, a FANA, and a morpholino.

Embodiment 366: The compound of any one of the preceding embodiments, wherein said first oligonucleotide is a gapmer, and wherein said second oligonucleotide is a modified second oligonucleotide comprising unmodified nucleosides and at least one but at most four modified nucleosides, wherein said unmodified nucleosides are unmodified RNA nucleosides, and wherein said modified nucleoside a tricyclic sugar moiety.

Embodiment 377: The compound of any one of the preceding embodiments, wherein said first oligonucleotide is a gapmer, and wherein said second oligonucleotide is a modified second oligonucleotide comprising unmodified nucleosides and at least one but at most four modified nucleosides, wherein said unmodified nucleosides are unmodified RNA nucleosides, and wherein said modified nucleoside is a sugar surrogate.

Embodiment 378: The compound of any one of the preceding embodiments, wherein said first oligonucleotide is a gapmer, and wherein said second oligonucleotide is a modified second oligonucleotide comprising unmodified nucleosides and at least one but at most four modified nucleosides, wherein said unmodified nucleosides are unmodified RNA nucleosides, and wherein said modified nucleoside is a sugar surrogate, and wherein said sugar surrogate is a morpholino.

Embodiment 379: The compound of any one of the preceding embodiments, wherein said first oligonucleotide is a gapmer, and wherein said second oligonucleotide is a modified oligonucleotide, and wherein said modified second oligonucleotide comprises at least one unmodified nucleoside and at least one modified nucleoside, wherein said at least one unmodified nucleoside is unmodified RNA sugar moiety, and wherein said modified nucleoside is selected from the group consisting of a 2′-MOE nucleoside, 2′-OMe nucleoside, an abc-DNA nucleoside, a LNA, a cET, cMOE, a CeNA, a HNA, a ANA, a FANA, a TNA, a tcDNA, a PNA, a GNA, a morpholino or a modified morpholino.

Embodiment 380: The compound of any one of the preceding embodiments, wherein said first oligonucleotide is a gapmer, and wherein said second oligonucleotide comprises at least one unmodified nucleoside and at least one modified nucleoside, wherein said at least one unmodified nucleoside is unmodified RNA sugar moiety, and wherein said modified nucleoside is selected from the group consisting of an a 2′-MOE nucleoside, abc-DNA nucleoside, a LNA, a CeNA, a HNA, a ANA, a FANA, a TNA, a tcDNA and a morpholino.

Embodiment 381: The compound of any one of the preceding embodiments, wherein said first oligonucleotide is a gapmer, and wherein said second oligonucleotide comprises at least one unmodified nucleoside and at least one modified nucleoside, wherein said at least one unmodified nucleoside is unmodified RNA sugar moiety, and wherein said modified nucleoside comprises a 2′-OMe sugar moiety.

Embodiment 382: The compound of any one of the preceding embodiments, wherein said first oligonucleotide is a gapmer, and wherein said second oligonucleotide comprises at least one unmodified nucleoside and at least one modified nucleoside, wherein said at least one unmodified nucleoside is unmodified RNA sugar moiety, and wherein said modified nucleoside comprises a 2′-MOE sugar moiety.

Embodiment 383: The compound of any one of the preceding embodiments, wherein said first oligonucleotide is a gapmer, and wherein said second oligonucleotide comprises at least one unmodified nucleoside and at least one modified nucleoside, wherein said at least one unmodified nucleoside is unmodified RNA sugar moiety, and wherein said modified nucleoside is a 2′-MOE nucleoside.

Embodiment 384: The compound of any one of the preceding embodiments, wherein said first oligonucleotide is a gapmer, and wherein said second oligonucleotide comprises at least one unmodified nucleoside and at least one modified nucleoside, wherein said at least one unmodified nucleoside is unmodified RNA sugar moiety, and wherein said modified nucleoside is an abc-DNA nucleoside.

Embodiment 385: The compound of any one of the preceding embodiments, wherein said first oligonucleotide is a gapmer, and wherein said second oligonucleotide comprises at least one unmodified nucleoside and at least one modified nucleoside, wherein said at least one unmodified nucleoside is unmodified RNA sugar moiety, and wherein said modified nucleoside is a LNA.

Embodiment 386: The compound of any one of the preceding embodiments, wherein said first oligonucleotide is a gapmer, and wherein said second oligonucleotide comprises at least one unmodified nucleoside and at least one modified nucleoside, wherein said at least one unmodified nucleoside is unmodified RNA sugar moiety, and wherein said modified nucleoside is a CeNA.

Embodiment 387: The compound of any one of the preceding embodiments, wherein said first oligonucleotide is a gapmer, and wherein said second oligonucleotide comprises at least one unmodified nucleoside and at least one modified nucleoside, wherein said at least one unmodified nucleoside is unmodified RNA sugar moiety, and wherein said modified nucleoside is a HNA.

Embodiment 388: The compound of any one of the preceding embodiments, wherein said first oligonucleotide is a gapmer, and wherein said second oligonucleotide comprises at least one unmodified nucleoside and at least one modified nucleoside, wherein said at least one unmodified nucleoside is unmodified RNA sugar moiety, and wherein said modified nucleoside is an ANA.

Embodiment 389: The compound of any one of the preceding embodiments, wherein said first oligonucleotide is a gapmer, and wherein said second oligonucleotide comprises at least one unmodified nucleoside and at least one modified nucleoside, wherein said at least one unmodified nucleoside is unmodified RNA sugar moiety, and wherein said modified nucleoside is a FANA.

Embodiment 390: The compound of any one of the preceding embodiments, wherein said first oligonucleotide is a gapmer, and wherein said second oligonucleotide comprises at least one unmodified nucleoside and at least one modified nucleoside, wherein said at least one unmodified nucleoside is unmodified RNA sugar moiety, and wherein said modified nucleoside is a TNA.

Embodiment 391: The compound of any one of the preceding embodiments, wherein said first oligonucleotide is a gapmer, and wherein said second oligonucleotide comprises at least one unmodified nucleoside and at least one modified nucleoside, wherein said at least one unmodified nucleoside is unmodified RNA sugar moiety, and wherein said modified nucleoside is a tcDNA.

Embodiment 392: The compound of any one of the preceding embodiments, wherein said first oligonucleotide is a gapmer, and wherein said second oligonucleotide comprises at least one unmodified nucleoside and at least one modified nucleoside, wherein said at least one unmodified nucleoside is unmodified RNA sugar moiety, and wherein said modified nucleoside is a morpholino.

Embodiment 393: The compound of any one of the preceding embodiments, wherein said first oligonucleotide is a gapmer, and wherein said second oligonucleotide comprises at least one unmodified nucleoside and at least one modified nucleoside, wherein said at least one unmodified nucleoside is unmodified RNA sugar moiety, and wherein said modified nucleoside is a modified morpholino.

Embodiment 394: The compound of any one of the preceding embodiments, wherein said first oligonucleotide is a gapmer, and wherein said second oligonucleotide is a modified second oligonucleotide comprising unmodified nucleosides and at least one but at most four modified nucleosides, wherein said unmodified nucleosides are unmodified RNA nucleosides, and wherein said modified nucleoside is selected from the group consisting of a 2′-MOE nucleoside, 2′-OMe nucleoside, an abc-DNA nucleoside, a LNA, a cET, cMOE, a CeNA, a HNA, a ANA, a FANA, a TNA, a tcDNA, a PNA, a GNA, a morpholino or a modified morpholino, wherein preferably said modified nucleosides are positioned at the 5′-end and/or at the 3′-end of said second oligonucleotide.

Embodiment 395: The compound of any one of the preceding embodiments, wherein said first oligonucleotide is a gapmer, and wherein said second oligonucleotide is a modified second oligonucleotide comprising unmodified nucleosides and at least one but at most four modified nucleosides, wherein said unmodified nucleosides are unmodified RNA nucleosides, and wherein said modified nucleoside is selected from the group consisting of a 2′-MOE nucleoside, 2′-OMe nucleoside, an abc-DNA nucleoside, a LNA, a CeNA, a HNA, a ANA, a FANA, a TNA, a tcDNA and a morpholino, wherein preferably said modified nucleosides are positioned at the 5′-end and/or at the 3′-end of said second oligonucleotide.

Embodiment 396: The compound of any one of the preceding embodiments, wherein said first oligonucleotide is a gapmer, and wherein said second oligonucleotide is a modified second oligonucleotide comprising unmodified nucleosides and at least one but at most four modified nucleosides, wherein said unmodified nucleosides are unmodified RNA nucleosides, and wherein said modified nucleoside comprises a 2′-OMe sugar moiety, wherein preferably said modified nucleosides are positioned at the 5′-end and/or at the 3′-end of said second oligonucleotide.

Embodiment 397: The compound of any one of the preceding embodiments, wherein said first oligonucleotide is a gapmer, and wherein said second oligonucleotide is a modified second oligonucleotide comprising unmodified nucleosides and at least one but at most four modified nucleosides, wherein said unmodified nucleosides are unmodified RNA nucleosides, and wherein said modified nucleoside comprises a 2′-MOE sugar moiety, wherein preferably said modified nucleosides are positioned at the 5′-end and/or at the 3′-end of said second oligonucleotide.

Embodiment 398: The compound of any one of the preceding embodiments, wherein said first oligonucleotide is a gapmer, and wherein said second oligonucleotide is a modified second oligonucleotide comprising unmodified nucleosides and at least one but at most four modified nucleosides, wherein said unmodified nucleosides are unmodified RNA nucleosides, and wherein said modified nucleoside is a 2′-MOE nucleoside, wherein preferably said modified nucleosides are positioned at the 5′-end and/or at the 3′-end of said second oligonucleotide.

Embodiment 399: The compound of any one of the preceding embodiments, wherein said first oligonucleotide is a gapmer, and wherein said second oligonucleotide is a modified second oligonucleotide comprising unmodified nucleosides and at least one but at most four modified nucleosides, wherein said unmodified nucleosides are unmodified RNA nucleosides, and wherein said modified nucleoside is an abc-DNA nucleoside, wherein preferably said modified nucleosides are positioned at the 5′-end and/or at the 3′-end of said second oligonucleotide.

Embodiment 400: The compound of any one of the preceding embodiments, wherein said first oligonucleotide is a gapmer, and wherein said second oligonucleotide is a modified second oligonucleotide comprising unmodified nucleosides and at least one but at most four modified nucleosides, wherein said unmodified nucleosides are unmodified RNA nucleosides, and wherein said modified nucleoside is a LNA, wherein preferably said modified nucleosides are positioned at the 5′-end and/or at the 3′-end of said second oligonucleotide.

Embodiment 401: The compound of any one of the preceding embodiments, wherein said first oligonucleotide is a gapmer, and wherein said second oligonucleotide is a modified second oligonucleotide comprising unmodified nucleosides and at least one but at most four modified nucleosides, wherein said unmodified nucleosides are unmodified RNA nucleosides, and wherein said modified nucleoside is a CeNA, wherein preferably said modified nucleosides are positioned at the 5′-end and/or at the 3′-end of said second oligonucleotide.

Embodiment 402: The compound of any one of the preceding embodiments, wherein said first oligonucleotide is a gapmer, and wherein said second oligonucleotide is a modified second oligonucleotide comprising unmodified nucleosides and at least one but at most four modified nucleosides, wherein said unmodified nucleosides are unmodified RNA nucleosides, and wherein said modified nucleoside is a HNA, wherein preferably said modified nucleosides are positioned at the 5′-end and/or at the 3′-end of said second oligonucleotide.

Embodiment 403: The compound of any one of the preceding embodiments, wherein said first oligonucleotide is a gapmer, and wherein said second oligonucleotide is a modified second oligonucleotide comprising unmodified nucleosides and at least one but at most four modified nucleosides, wherein said unmodified nucleosides are unmodified RNA nucleosides, and wherein said modified nucleoside is an ANA, wherein preferably said modified nucleosides are positioned at the 5′-end and/or at the 3′-end of said second oligonucleotide.

Embodiment 404: The compound of any one of the preceding embodiments, wherein said first oligonucleotide is a gapmer, and wherein said second oligonucleotide is a modified second oligonucleotide comprising unmodified nucleosides and at least one but at most four modified nucleosides, wherein said unmodified nucleosides are unmodified RNA nucleosides, and wherein said modified nucleoside is a FANA, wherein preferably said modified nucleosides are positioned at the 5′-end and/or at the 3′-end of said second oligonucleotide.

Embodiment 405: The compound of any one of the preceding embodiments, wherein said first oligonucleotide is a gapmer, and wherein said second oligonucleotide is a modified second oligonucleotide comprising unmodified nucleosides and at least one but at most four modified nucleosides, wherein said unmodified nucleosides are unmodified RNA nucleosides, and wherein said modified nucleoside is a TNA, wherein preferably said modified nucleosides are positioned at the 5′-end and/or at the 3′-end of said second oligonucleotide.

Embodiment 406: The compound of any one of the preceding embodiments, wherein said first oligonucleotide is a gapmer, and wherein said second oligonucleotide is a modified second oligonucleotide comprising unmodified nucleosides and at least one but at most four modified nucleosides, wherein said unmodified nucleosides are unmodified RNA nucleosides, and wherein said modified nucleoside is a tcDNA, wherein preferably said modified nucleosides are positioned at the 5′-end and/or at the 3′-end of said second oligonucleotide.

Embodiment 407: The compound of any one of the preceding embodiments, wherein said first oligonucleotide is a gapmer, and wherein said second oligonucleotide is a modified second oligonucleotide comprising unmodified nucleosides and at least one but at most four modified nucleosides, wherein said unmodified nucleosides are unmodified RNA nucleosides, and wherein said modified nucleoside is a morpholino, wherein preferably said modified nucleosides are positioned at the 5′-end and/or at the 3′-end of said second oligonucleotide.

Embodiment 408: The compound of any one of the preceding embodiments, wherein said first oligonucleotide is a gapmer, and wherein said second oligonucleotide is a modified second oligonucleotide comprising unmodified nucleosides and at least one but at most four modified nucleosides, wherein said unmodified nucleosides are unmodified RNA nucleosides, and wherein said modified nucleoside is a modified morpholino, wherein preferably said modified nucleosides are positioned at the 5′-end and/or at the 3′-end of said second oligonucleotide.

Embodiment 409: The compound of any one of the preceding embodiments, wherein said first oligonucleotide is a gapmer, and wherein said second oligonucleotide is a modified second oligonucleotide comprising unmodified nucleosides and one or two modified nucleosides, wherein said unmodified nucleosides are unmodified RNA nucleosides, and wherein said modified nucleoside is selected from the group consisting of a 2′-MOE nucleoside, 2′-OMe nucleoside, an abc-DNA nucleoside, a LNA, a cET, cMOE, a CeNA, a HNA, a ANA, a FANA, a TNA, a tcDNA, a PNA, a GNA, a morpholino or a modified morpholino, and wherein preferably said one or two modified nucleosides are the nucleosides at the 3′end of said second oligonucleotide.

Embodiment 410: The compound of any one of the preceding embodiments, wherein said first oligonucleotide is a gapmer, and wherein said second oligonucleotide is a modified second oligonucleotide comprising unmodified nucleosides and one or two modified nucleosides, wherein said unmodified nucleosides are unmodified RNA nucleosides, and wherein said modified nucleoside is selected from the group consisting of a 2′-MOE nucleoside, 2′-OMe nucleoside, an abc-DNA nucleoside, a LNA, a CeNA, a HNA, a ANA, a FANA, a TNA, a tcDNA and a morpholino, and wherein preferably said one or two modified nucleosides are the nucleosides at the 3′end of said second oligonucleotide.

Embodiment 411: The compound of any one of the preceding embodiments, wherein said first oligonucleotide is a gapmer, and wherein said second oligonucleotide is a modified second oligonucleotide comprising unmodified nucleosides and one or two modified nucleosides, wherein said unmodified nucleosides are unmodified RNA nucleosides, and wherein said modified nucleoside comprises a 2′-OMe sugar moiety, and wherein preferably said one or two modified nucleosides are the nucleosides at the 3′end of said second oligonucleotide.

Embodiment 412: The compound of any one of the preceding embodiments, wherein said first oligonucleotide is a gapmer, and wherein said second oligonucleotide is a modified second oligonucleotide comprising unmodified nucleosides and one or two modified nucleosides, wherein said unmodified nucleosides are unmodified RNA nucleosides, and wherein said modified nucleoside comprises a 2′-MOE sugar moiety, and wherein preferably said one or two modified nucleosides are the nucleosides at the 3′end of said second oligonucleotide.

Embodiment 413: The compound of any one of the preceding embodiments, wherein said first oligonucleotide is a gapmer, and wherein said second oligonucleotide is a modified second oligonucleotide comprising unmodified nucleosides and one or two modified nucleosides, wherein said unmodified nucleosides are unmodified RNA nucleosides, and wherein said modified nucleoside is a 2′-MOE nucleoside, and wherein preferably said one or two modified nucleosides are the nucleosides at the 3′end of said second oligonucleotide.

Embodiment 414: The compound of any one of the preceding embodiments, wherein said first oligonucleotide is a gapmer, and wherein said second oligonucleotide is a modified second oligonucleotide comprising unmodified nucleosides and one or two modified nucleosides, wherein said unmodified nucleosides are unmodified RNA nucleosides, and wherein said modified nucleoside is an abc-DNA nucleoside, and wherein preferably said one or two modified nucleosides are the nucleosides at the 3′end of said second oligonucleotide.

Embodiment 415: The compound of any one of the preceding embodiments, wherein said first oligonucleotide is a gapmer, and wherein said second oligonucleotide is a modified second oligonucleotide comprising unmodified nucleosides and one or two modified nucleosides, wherein said unmodified nucleosides are unmodified RNA nucleosides, and wherein said modified nucleoside is a LNA, and wherein preferably said one or two modified nucleosides are the nucleosides at the 3′end of said second oligonucleotide.

Embodiment 416: The compound of any one of the preceding embodiments, wherein said first oligonucleotide is a gapmer, and wherein said second oligonucleotide is a modified second oligonucleotide comprising unmodified nucleosides and one or two modified nucleosides, wherein said unmodified nucleosides are unmodified RNA nucleosides, and wherein said modified nucleoside is a CeNA, and wherein preferably said one or two modified nucleosides are the nucleosides at the 3′end of said second oligonucleotide.

Embodiment 417: The compound of any one of the preceding embodiments, wherein said first oligonucleotide is a gapmer, and wherein said second oligonucleotide is a modified second oligonucleotide comprising unmodified nucleosides and one or two modified nucleosides, wherein said unmodified nucleosides are unmodified RNA nucleosides, and wherein said modified nucleoside is a HNA, and wherein preferably said one or two modified nucleosides are the nucleosides at the 3′end of said second oligonucleotide.

Embodiment 418: The compound of any one of the preceding embodiments, wherein said first oligonucleotide is a gapmer, and wherein said second oligonucleotide is a modified second oligonucleotide comprising unmodified nucleosides and one or two modified nucleosides, wherein said unmodified nucleosides are unmodified RNA nucleosides, and wherein said modified nucleoside is an ANA, and wherein preferably said one or two modified nucleosides are the nucleosides at the 3′end of said second oligonucleotide.

Embodiment 419: The compound of any one of the preceding embodiments, wherein said first oligonucleotide is a gapmer, and wherein said second oligonucleotide is a modified second oligonucleotide comprising unmodified nucleosides and one or two modified nucleosides, wherein said unmodified nucleosides are unmodified RNA nucleosides, and wherein said modified nucleoside is a FANA, and wherein preferably said one or two modified nucleosides are the nucleosides at the 3′end of said second oligonucleotide.

Embodiment 420: The compound of any one of the preceding embodiments, wherein said first oligonucleotide is a gapmer, and wherein said second oligonucleotide is a modified second oligonucleotide comprising unmodified nucleosides and one or two modified nucleosides, wherein said unmodified nucleosides are unmodified RNA nucleosides, and wherein said modified nucleoside is a TNA, and wherein preferably said one or two modified nucleosides are the nucleosides at the 3′end of said second oligonucleotide.

Embodiment 421: The compound of any one of the preceding embodiments, wherein said first oligonucleotide is a gapmer, and wherein said second oligonucleotide is a modified second oligonucleotide comprising unmodified nucleosides and one or two modified nucleosides, wherein said unmodified nucleosides are unmodified RNA nucleosides, and wherein said modified nucleoside is a tcDNA, and wherein preferably said one or two modified nucleosides are the nucleosides at the 3′end of said second oligonucleotide.

Embodiment 422: The compound of any one of the preceding embodiments, wherein said first oligonucleotide is a gapmer, and wherein said second oligonucleotide is a modified second oligonucleotide comprising unmodified nucleosides and one or two modified nucleosides, wherein said unmodified nucleosides are unmodified RNA nucleosides, and wherein said modified nucleoside is a morpholino, and wherein preferably said one or two modified nucleosides are the nucleosides at the 3′end of said second oligonucleotide.

Embodiment 423: The compound of any one of the preceding embodiments, wherein said first oligonucleotide is a gapmer, and wherein said second oligonucleotide is a modified second oligonucleotide comprising unmodified nucleosides and one or two modified nucleosides, wherein said unmodified nucleosides are unmodified RNA nucleosides, and wherein said modified nucleoside is a modified morpholino, and wherein preferably said one or two modified nucleosides are the nucleosides at the 3′end of said second oligonucleotide.

Embodiment 424: The compound of any one of the preceding embodiments, wherein at least 10% of the nucleosides of the first oligonucleotide are abc-DNA nucleosides.

Embodiment 425: The compound of any one of the preceding embodiments, wherein at least 20% of the nucleosides of the first oligonucleotide are abc-DNA nucleosides.

Embodiment 426: The compound of any one of the preceding embodiments, wherein at least 30% of the nucleosides of the first oligonucleotide are abc-DNA nucleosides.

Embodiment 427: The compound of any one of the preceding embodiments, wherein at least 40% of the nucleosides of the first oligonucleotide are abc-DNA nucleosides.

Embodiment 428: The compound of any one of the preceding embodiments, wherein at least 50% of the nucleosides of the first oligonucleotide are abc-DNA nucleosides.

Embodiment 429: The compound of any one of the preceding embodiments, wherein at least 60% of the nucleosides of the first oligonucleotide are abc-DNA nucleosides.

Embodiment 430: The compound of any one of the preceding embodiments, wherein at least 70% of the nucleosides of the first oligonucleotide are abc-DNA nucleosides.

Embodiment 431: The compound of any one of the preceding embodiments, wherein at least 80% of the nucleosides of the first oligonucleotide are abc-DNA nucleosides.

Embodiment 432: The compound of any one of the preceding embodiments, wherein at least 90% of the nucleosides of the first oligonucleotide are abc-DNA nucleosides.

Embodiment 433: The compound of any one of the preceding embodiments, wherein 100% of the nucleosides of the first oligonucleotide are abc-DNA nucleosides.

Embodiment 434: The compound of any one of the preceding embodiments, wherein said first oligonucleotide comprises at least 8 and at most 50 nucleotides.

Embodiment 435: The compound of any one of the preceding embodiments, wherein the first oligonucleotide comprises at least 10 and at most 40 nucleotides.

Embodiment 436: The compound of any one of the preceding embodiments, wherein the first oligonucleotide comprises at least 12 and at most 30 nucleotides.

Embodiment 437: The compound of any one of the preceding embodiments, wherein the first oligonucleotide comprises at least 12 and at most 25 nucleotides.

Embodiment 438: The compound of any one of the preceding embodiments, wherein the first oligonucleotide comprises at least 15 and at most 20 nucleotides.

Embodiment 439: The compound of any one of the preceding embodiments, wherein at most five, preferably at most four, preferably at most three of all internucleoside linkages of said first oligonucleotide are phosphorothioate internucleoside linkages, and the remaining internucleoside linkages of said first oligonucleotide are phosphodiester linkages.

Embodiment 440: The compound of any one of the preceding embodiments, wherein at most two, preferably one or none of all internucleoside linkages of said first oligonucleotide are phosphorothioate internucleoside linkages, and the remaining internucleoside linkages of said first oligonucleotide are phosphodiester linkages.

Embodiment 441: The compound of any one of the preceding embodiments, wherein all internucleoside linkages of said first oligonucleotide are phosphodiester linkages.

Embodiment 442: The compound of any one of the preceding embodiments, wherein at least 70% of the sugar moieties of said second oligonucleotide are unmodified DNA sugar moieties, preferably wherein at least 80%, more preferably at least 90%, more preferably at least 95%, and yet more preferably 100% of the sugar moieties of said second oligonucleotide are unmodified DNA sugar moieties, and wherein preferably at most three of all internucleoside linkages of said second oligonucleotide are phosphorothioate internucleoside linkages, wherein preferably said phosphorothioate internucleoside linkages are positioned at the 5′-end and/or at the 3′-end of said second oligonucleotide.

Embodiment 443: The compound of any one of the preceding embodiments, wherein at least 10%, preferably 20%, further preferably 30% of the sugar moieties of the second oligonucleotide are unmodified DNA sugar moieties.

Embodiment 444: The compound of any one of the preceding embodiments, wherein at least 40% of the sugar moieties of the second oligonucleotide are unmodified DNA sugar moieties.

Embodiment 445: The compound of any one of the preceding embodiments, wherein at least 50% of the sugar moieties of the second oligonucleotide are unmodified DNA sugar moieties.

Embodiment 446: The compound of any one of the preceding embodiments, wherein at least 60% of the sugar moieties of the second oligonucleotide are unmodified DNA sugar moieties.

Embodiment 447: The compound of any one of the preceding embodiments, wherein at least at least 70% of the nucleosides of the first oligonucleotide are abc-DNA nucleosides and at least 70% of the sugar moieties of the second oligonucleotide are unmodified DNA sugar moieties.

Embodiment 448: The compound of any one of the preceding embodiments, wherein at least at least 80% of the nucleosides of the first oligonucleotide are abc-DNA nucleosides and at least 80% of the sugar moieties of the second oligonucleotide are unmodified DNA sugar moieties.

Embodiment 449: The compound of any one of the preceding embodiments, wherein at least at least 90% of the nucleosides of the first oligonucleotide are abc-DNA nucleosides and at least 90% of the sugar moieties of the second oligonucleotide are unmodified DNA sugar moieties.

Embodiment 450: The compound of any one of the preceding embodiments, wherein all of the nucleosides of the first oligonucleotide are abc-DNA nucleosides and all the sugar moieties of the second oligonucleotide are unmodified DNA sugar moieties.

Embodiment 451: The compound of any one of the preceding embodiments, wherein at least at least 70% of the nucleosides of the first oligonucleotide are abc-DNA nucleosides and at least 70% of the nucleosides of the second oligonucleotide are unmodified DNA nucleosides.

Embodiment 452: The compound of any one of the preceding embodiments, wherein at least at least 80% of the nucleosides of the first oligonucleotide are abc-DNA nucleosides and at least 80% of nucleosides of the second oligonucleotide are unmodified DNA nucleosides.

Embodiment 453: The compound of any one of the preceding embodiments, wherein at least at least 90% of the nucleosides of the first oligonucleotide are abc-DNA nucleosides and at least 90% of the nucleosides of the second oligonucleotide are unmodified DNA nucleosides.

Embodiment 454: The compound of any one of the preceding embodiments, wherein all of the nucleosides of the first oligonucleotide are abc-DNA nucleosides and all the nucleosides of the second oligonucleotide are unmodified DNA nucleosides.

Embodiment 455: The compound of any one of the preceding embodiments, wherein each sugar moiety of said second oligonucleotide is an unmodified DNA sugar moiety, wherein at most four of all internucleoside linkages of said second oligonucleotide are phosphorothioate internucleoside linkages, wherein preferably said phosphorothioate internucleoside linkages are positioned at the 5′-end and/or at the 3′-end of said second oligonucleotide.

Embodiment 456: The compound of any one of the preceding embodiments, wherein each sugar moiety of said second oligonucleotide is an unmodified DNA sugar moiety, wherein at most three of all internucleoside linkages of said second oligonucleotide are phosphorothioate internucleoside linkages and the remaining internucleoside linkages of said first oligonucleotide are phosphodiester linkages, wherein preferably said phosphorothioate internucleoside linkages are positioned at the 5′-end and/or at the 3′-end of said second oligonucleotide.

Embodiment 457: The compound of any one of the preceding embodiments, wherein each sugar moiety of said second oligonucleotide is an unmodified DNA sugar moiety, wherein at most two of all internucleoside linkages of said second oligonucleotide are phosphorothioate internucleoside linkages and the remaining internucleoside linkages of said first oligonucleotide are phosphodiester linkages, and wherein preferably said one or two phosphorothioate internucleoside linkages are at the 3′end of said second oligonucleotide.

Embodiment 458: The compound of any one of the preceding embodiments, wherein each sugar moiety of said second oligonucleotide is an unmodified DNA sugar moiety, wherein each internucleoside linkage of said second oligonucleotide is a phosphodiester linkage.

Embodiment 459: The compound of any one of the preceding embodiments, wherein each nucleotide of said second oligonucleotide is an unmodified DNA nucleotide.

Embodiment 460: The compound of any one of the preceding embodiments, wherein said second oligonucleotide comprises at least one modified sugar moiety selected from the group consisting of (i) a non-bicyclic modified sugar moiety comprising a furanosyl ring with one or more acyclic substituent at the 2′, 4′, and/or 5′ position, (ii) a bicyclic sugar moiety, and (iii) a sugar surrogate.

Embodiment 461: The compound of any one of the preceding embodiments, wherein said second oligonucleotide consists of modified nucleosides selected from the group consisting of (i) a non-bicyclic modified sugar moiety comprising a furanosyl ring with one or more acyclic substituent at the 2′, 4′, and/or 5′ position, (ii) a bicyclic sugar moiety, (iii) a sugar surrogate.

Embodiment 462: The compound of any one of the preceding embodiments, wherein said second oligonucleotide comprises at least 70% modified sugar moieties, preferably at least 80% modified sugar moieties, more preferably at least 90% modified sugar moieties, most preferably 100% modified sugar moieties, preferably wherein said modified sugar moieties are non-bicyclic modified sugar moieties comprising a furanosyl ring with one or more acyclic substituent at the 2′, 4′, and/or 5′ position.

Embodiment 463: The compound of any one of the preceding embodiments, wherein said second oligonucleotide comprises at least 70% modified sugar moieties, preferably at least 80% modified sugar moieties, more preferably at least 90% modified sugar moieties, most preferably 100% modified sugar moieties, preferably wherein said modified sugar moieties are bicyclic sugar moieties.

Embodiment 464: The compound of any one of the preceding embodiments, wherein said second oligonucleotide comprises at least 70% modified sugar moieties, preferably at least 80% modified sugar moieties, more preferably at least 90% modified sugar moieties, most preferably 100% modified sugar moieties, preferably wherein said modified sugar moieties are sugar surrogates.

Embodiment 465: The compound of any one of the preceding embodiments, wherein said second oligonucleotide consists of modified nucleosides selected from the group consisting of a 2′-MOE nucleoside, 2′-OMe nucleoside, an abc-DNA nucleoside, a LNA, a cET, cMOE, a CeNA, a HNA, a ANA, a FANA, a TNA, a tcDNA, a PNA, a GNA, a morpholino and a modified morpholino.

Embodiment 466: The compound of any one of the preceding embodiments, wherein said second oligonucleotide consists of modified nucleosides, wherein said modified nucleosides are selected from the group consisting of a 2′-MOE nucleoside, 2′-OMe nucleoside, an abc-DNA nucleoside, a FANA, and a morpholino.

Embodiment 467: The compound of any one of the preceding embodiments, wherein said first oligonucleotide is a gapmer comprising a gap, a 5′ wing and a 3′ wing.

Embodiment 468: The compound of any one of the preceding embodiments, wherein said gap of said gapmer comprises unmodified DNA sugar moieties.

Embodiment 469: The compound of any one of the preceding embodiments, wherein gap of said gapmer consists of unmodified DNA sugar moieties.

Embodiment 470: The compound of any one of the preceding embodiments, wherein said 5′ wing of said gapmer comprises at least one abc-DNA nucleoside; and/or wherein said 3′ wing of said gapmer comprises at least one abc-DNA nucleoside.

Embodiment 471: The compound of any one of the preceding embodiments, wherein said 5′ wing of said gapmer comprises at least one abc-DNA nucleoside, preferably wherein said 5′ wing of said gapmer comprises at least two abc-DNA nucleosides, preferably at least three abc-DNA nucleosides, yet more preferably at least four abc-DNA nucleosides, and again more preferably at least 5 abc-DNA nucleosides; preferably wherein said abc-DNA nucleosides are contiguous.

Embodiment 472: The compound of any one of the preceding embodiments, wherein the 5′ wing of said gapmer consists of abc-DNA nucleosides.

Embodiment 473: The compound of any one of the preceding embodiments, wherein said 3′ wing of said gapmer comprises at least one abc-DNA nucleoside, preferably wherein said 3′ wing of said gapmer comprises at least two abc-DNA nucleosides, preferably at least three abc-DNA nucleosides, yet more preferably at least four abc-DNA nucleosides, and again more preferably at least 5 abc-DNA nucleosides; preferably wherein said abc-DNA nucleosides are contiguous.

Embodiment 474: The compound of any one of the preceding embodiments, wherein the 3′ wing of said gapmer consists of abc-DNA nucleosides.

Embodiment 475: The compound of any one of the preceding embodiments, wherein said gap of said gapmer consists of unmodified DNA sugar moieties; said 5′ wing of said gapmer consists of abc-DNA nucleosides; and said 3′ wing of said gapmer consists of abc-DNA nucleosides.

Embodiment 476: The compound of any one of the preceding embodiments, wherein said gap comprises at least five unmodified DNA sugar moieties, preferably at least six unmodified DNA sugar moieties, preferably at least seven unmodified DNA sugar moieties, preferably at least eight unmodified DNA sugar moieties, yet preferably at least nine unmodified DNA nucleosides, yet more preferably at least ten unmodified DNA sugar moieties.

Embodiment 477: The compound of any one of the preceding embodiments, wherein said 5′ wing of said gapmer comprises at least four abc-DNA nucleosides, and wherein said 3′ wing of said gapmer comprises at least four abc-DNA nucleosides.

Embodiment 478: The compound of any one of the preceding embodiments, wherein said gap of said gapmer comprises at least five unmodified DNA sugar moieties, said 5′ wing of said gapmer comprises at least four abc-DNA nucleosides, and wherein said 3′ wing of said gapmer comprises at least four abc-DNA nucleosides.

Embodiment 479: The compound of any one of the preceding embodiments, wherein at least 20% of the internucleoside linkages within said gap of said gapmer are phosphodiester internucleoside linkages.

Embodiment 480: The compound of any one of the preceding embodiments, wherein at most eight, at most seven, at most six, at most five, at most four, at most three, at most two, or at most one of all of the internucleoside linkages within said gap of said gapmer are phosphorothioate internucleoside linkages.

Embodiment 481: The compound of any one of the preceding embodiments, wherein at most 30%, preferably 20%, further preferably at most 10% of the internucleoside linkages within said gap of said gapmer are phosphorothioate internucleoside linkages.

Embodiment 482: The compound of any one of the preceding embodiments, wherein at least 40% of the internucleoside linkages within said gap of said gapmer are phosphodiester internucleoside linkages.

Embodiment 483: The compound of any one of the preceding embodiments, wherein at least 50% of the internucleoside linkages within said gap of said gapmer are phosphodiester internucleoside linkages.

Embodiment 484: The compound of any one of the preceding embodiments, wherein at least 60% of the internucleoside linkages within said gap of said gapmer are phosphodiester internucleoside linkages.

Embodiment 485: The compound of any one of the preceding embodiments, wherein at least 70%, preferably 80%, of the internucleoside linkages within said gap of said gapmer are phosphodiester internucleoside linkages.

Embodiment 486: The compound of any one of the preceding embodiments, wherein at least 90%, of the internucleoside linkages within said gap of said gapmer are phosphodiester internucleoside linkages.

Embodiment 487: The compound of any one of the preceding embodiments, wherein at least 95% of the internucleoside linkages within said gap of said gapmer are phosphodiester internucleoside linkages.

Embodiment 488: The compound of any one of the preceding embodiments, wherein all of the internucleoside linkages within said gap of said gapmer are phosphodiester internucleoside linkages.

Embodiment 489: The compound of any one of the preceding embodiments, wherein said gap comprise at least two phosphodiester internucleoside linkages, preferably at most one phosphodiester internucleoside linkages.

Embodiment 490: The compound of any one of the preceding embodiments, wherein at least 70% of the internucleoside linkages within said 5′ wing of said gapmer are phosphodiester internucleoside linkages.

Embodiment 491: The compound of any one of the preceding embodiments, wherein at least 70% of the internucleoside linkages within said 3′ wing of said gapmer are phosphodiester internucleoside linkages.

Embodiment 492: The compound of any one of the preceding embodiments, wherein at least 80%, preferably 90%, more preferably 100% of said internucleoside linkages within said 5′ wing of said gapmer are phosphodiester internucleoside linkages.

Embodiment 493: The compound of any one of the preceding embodiments, wherein at least 80%, preferably 90%, more preferably 100% of said internucleoside linkages within said 3′ wing of said gapmer are phosphodiester internucleoside linkages.

Embodiment 493A: The compound of any one of the preceding embodiments, wherein 100% of said internucleoside linkages within said 3′ wing and/or 5′ wing of said gapmer are phosphodiester internucleoside linkages.

Embodiment 494: The compound of any one of the preceding embodiments, wherein all of said internucleoside linkages within said 5′ wing of said gapmer are phosphodiester internucleoside linkages; all of said internucleoside linkages within said 3′ wing of said gapmer are phosphodiester internucleoside linkages; and at least 50%, preferably 60%, more preferably 70%, more preferably 80% and most preferably at least 90% of said internucleoside linkages of said gap are phosphodiester linkages.

Embodiment 495: The compound of any one of the preceding embodiments, wherein said 5′ wing of said gapmer comprises at least two abc-DNA nucleosides, preferably at least three abc-DNA nucleosides, yet more preferably at least four abc-DNA nucleosides, and again more preferably at least 5 abc-DNA nucleosides, and wherein at least one of the internucleoside linkages between said at least two, preferably at least three, further preferably at least four, and again further preferably at least five, abc-DNA nucleosides are phosphodiester linkages.

Embodiment 496: The compound of any one of the preceding embodiments, wherein said 5′ wing of said gapmer comprises at least three abc-DNA nucleosides, preferably at least four abc-DNA nucleosides, and further preferably at least 5 abc-DNA nucleosides, and wherein at least two of the internucleoside linkages between said at least three, preferably at least four, again further preferably at least five, abc-DNA nucleosides are phosphodiester linkages.

Embodiment 497: The compound of any one of the preceding embodiments, wherein said 5′ wing of said gapmer comprises at least four abc-DNA nucleosides, preferably at least 5 abc-DNA nucleosides, and wherein at least three of the internucleoside linkages between said at least four, preferably at least five, abc-DNA nucleosides are phosphodiester linkages.

Embodiment 498: The compound of any one of the preceding embodiments, wherein said 5′ wing of said gapmer comprises at least 5 abc-DNA nucleosides, and wherein at least four of the internucleoside linkages between said at least five abc-DNA nucleosides are phosphodiester linkages.

Embodiment 499: The compound of any one of the preceding embodiments, wherein all of the internucleoside linkages between said at least one abc-DNA nucleosides are phosphodiester linkages.

Embodiment 500: The compound of any one of the preceding embodiments, wherein said 5′ wing of said gapmer comprises at least one abc-DNA nucleoside, and said 3′ wing of said gapmer comprises at least one 2′-MOE nucleoside, preferably at least two 2′-MOE nucleosides, more preferably at least three 2′-MOE nucleosides, more preferably at least four 2′-MOE nucleosides, more preferably at least five 2′-MOE nucleosides.

Embodiment 501: The compound of any one of the preceding embodiments, wherein said 5′ wing of said gapmer comprises at least one abc-DNA nucleoside, and said 3′ wing of said gapmer comprises at least two 2′-MOE nucleosides, wherein the internucleoside linkages between said 2′-MOE nucleosides of said 3′ wing are phosphorothioate internucleoside linkages.

Embodiment 502: The compound of any one of the preceding embodiments, wherein said 5′ wing of said gapmer comprises at least one abc-DNA nucleoside, and said 3′ wing of said gapmer comprises at least one LNA nucleoside, preferably at least two LNA nucleosides, more preferably at least three LNA nucleosides, more preferably at least four LNA nucleosides, more preferably at least five LNA nucleosides.

Embodiment 503: The compound of any one of the preceding embodiments, wherein said 5′ wing of said gapmer comprises at least one abc-DNA nucleoside, and said 3′ wing of said gapmer comprises at least two LNA nucleosides, wherein the internucleoside linkages between said LNA nucleosides of said 3′ wing are phosphorothioate internucleoside linkages.

Embodiment 504: The compound of any one of the preceding embodiments, wherein said 5′ wing of said gapmer comprises at least one abc-DNA nucleoside, and said 3′ wing of said gapmer comprises at least one FANA nucleoside, preferably at least two FANA nucleosides, more preferably at least three FANA nucleosides, more preferably at least four FANA nucleosides, more preferably at least five FANA nucleosides.

Embodiment 505: The compound of any one of the preceding embodiments, wherein said 5′ wing of said gapmer comprises at least one abc-DNA nucleoside, and said 3′ wing of said gapmer comprises at least two FANA nucleosides, wherein the internucleoside linkages between said FANA nucleosides of said 3′ wing are phosphorothioate internucleoside linkages.

Embodiment 506: The compound of any one of the preceding embodiments, wherein said 3′ wing of said gapmer comprises at least two abc-DNA nucleosides, preferably at least three abc-DNA nucleosides, yet more preferably at least four abc-DNA nucleosides, and again more preferably at least 5 abc-DNA nucleosides, and wherein at least one of the internucleoside linkages between said at least two, preferably at least three, further preferably at least four, and again further preferably at least five, abc-DNA nucleosides are phosphodiester linkages.

Embodiment 507: The compound of any one of the preceding embodiments, wherein said 3′ wing of said gapmer comprises at least three abc-DNA nucleosides, preferably at least four abc-DNA nucleosides, and further preferably at least 5 abc-DNA nucleosides, and wherein at least two of the internucleoside linkages between said at least three, preferably at least four, again further preferably at least five, abc-DNA nucleosides are phosphodiester linkages.

Embodiment 508: The compound of any one of the preceding embodiments, wherein said 3′ wing of said gapmer comprises at least four abc-DNA nucleosides, preferably at least 5 abc-DNA nucleosides, and wherein at least three of the internucleoside linkages between said at least four, preferably at least five, abc-DNA nucleosides are phosphodiester linkages.

Embodiment 509: The compound of any one of the preceding embodiments, wherein said 3′ wing of said gapmer comprises at least 5 abc-DNA nucleosides, and wherein at least four of the internucleoside linkages between said at least five abc-DNA nucleosides are phosphodiester linkages.

Embodiment 510: The compound of any one of the preceding embodiments, wherein all of the internucleoside linkages between said at least one abc-DNA nucleosides are phosphodiester linkages.

Embodiment 511: The compound of any one of the preceding embodiments, wherein said 3′ wing of said gapmer comprises at least one abc-DNA nucleoside, and said 5′ wing of said gapmer comprises at least one 2′-MOE nucleoside, preferably at least two 2′-MOE nucleosides, more preferably at least three 2′-MOE nucleosides, more preferably at least four 2′-MOE nucleosides, more preferably at least five 2′-MOE nucleosides.

Embodiment 512: The compound of any one of the preceding embodiments, wherein said 3′ wing of said gapmer comprises at least one abc-DNA nucleoside, and said 5′ wing of said gapmer comprises at least two 2′-MOE nucleosides, wherein the internucleoside linkages between said 2′-MOE nucleosides of said 5′ wing are phosphorothioate internucleoside linkages.

Embodiment 513: The compound of any one of the preceding embodiments, wherein said 3′ wing of said gapmer comprises at least one abc-DNA nucleoside, and said 5′ wing of said gapmer comprises at least one LNA nucleoside, preferably at least two LNA nucleosides, more preferably at least three LNA nucleosides, more preferably at least four LNA nucleosides, more preferably at least five LNA nucleosides.

Embodiment 514: The compound of any one of the preceding embodiments, wherein said 3′ wing of said gapmer comprises at least one abc-DNA nucleoside, and said 5′ wing of said gapmer comprises at least two LNA nucleosides, wherein the internucleoside linkages between said LNA nucleosides of said 5′ wing are phosphorothioate internucleoside linkages.

Embodiment 515: The compound of any one of the preceding embodiments, wherein said 3′ wing of said gapmer comprises at least one abc-DNA nucleoside, and said 5′ wing of said gapmer comprises at least one FANA nucleoside, preferably at least two FANA nucleosides, more preferably at least three FANA nucleosides, more preferably at least four FANA nucleosides, more preferably at least five FANA nucleosides.

Embodiment 516: The compound of any one of the preceding embodiments, wherein said 3′ wing of said gapmer comprises at least one abc-DNA nucleoside, and said 5′ wing of said gapmer comprises at least two FANA nucleosides, wherein the internucleoside linkages between said FANA nucleosides of said 5′ wing are phosphorothioate internucleoside linkages.

Embodiment 517: The compound of any one of the preceding embodiments, wherein said 3′ wing of said gapmer and said 5′ wing of said gapmer both comprise at least one abc-DNA nucleoside.

Embodiment 518: The compound of any one of the preceding embodiments, wherein said 3′ wing of the gapmer and said 5′ wing of said gapmer both comprise at least two abc-DNA nucleosides, preferably at least three abc-DNA nucleosides, yet more preferably at least four abc-DNA nucleosides, and again more preferably at least 5 abc-DNA nucleosides.

Embodiment 519: The compound of any one of the preceding embodiments, wherein the 3′ wing of the gapmer and the 5′ wing of the gapmer both comprise at least two abc-DNA nucleosides, preferably at least three abc-DNA nucleosides, yet more preferably at least four abc-DNA nucleosides, and yet more preferably at least 5 abc-DNA nucleosides; and wherein at least one of the internucleoside linkages between said at least two, preferably at least three, further preferably at least four, and again further preferably at least five, abc-DNA nucleosides are phosphodiester linkages.

Embodiment 520: The compound of any one of the preceding embodiments, wherein the internucleoside linkage between said 5′ wing and said gap of said gapmer is a phosphodiester internucleoside linkage.

Embodiment 521: The compound of any one of the preceding embodiments, wherein the internucleoside linkage between said 3′ wing and said gap of said gapmer a phosphodiester internucleoside linkage.

Embodiment 522: The compound of any one of the preceding embodiments, wherein said second oligonucleotide comprises at least one unmodified nucleoside.

Embodiment 523: The compound of any one of the preceding embodiments, wherein at least one unmodified nucleoside is an unmodified RNA nucleoside.

Embodiment 524: The compound of any one of the preceding embodiments, wherein said second oligonucleotide consists of unmodified RNA nucleosides.

Embodiment 525: The compound of any one of the preceding embodiments, wherein said second oligonucleotide further comprises at least one modified nucleoside, preferably wherein said at least one modified nucleoside is an abcDNA nucleoside or an MOE nucleoside.

Embodiment 526: The compound of any one of the preceding embodiments, wherein said at least one modified nucleoside is an abcDNA nucleoside.

Embodiment 527: The compound of any one of the preceding embodiments, wherein said at least one modified nucleoside is an MOE nucleoside.

Embodiment 528: The compound of any one of the preceding embodiments, wherein said at least one modified nucleoside is positioned at the 5′ end and/or the 3′ end of said second oligonucleotide.

Embodiment 529: The compound of any one of the preceding embodiments, wherein said second oligonucleotide comprises at least two modified nucleosides, wherein said modified nucleosides are positioned at the 5′ end of the second oligonucleotide and are contiguous.

Embodiment 530: The compound of any one of the preceding embodiments, wherein said second oligonucleotide comprises at least two modified nucleosides, wherein said modified nucleosides are positioned at the 3′ end of the second oligonucleotide and are contiguous.

Embodiment 531: The compound of any one of the preceding embodiments, wherein said second oligonucleotide comprises at least two modified nucleosides, wherein a first of said modified nucleosides is positioned at the 5′ end of the second oligonucleotide; and a second of said modified nucleosides is positioned at the 3′ end of the second oligonucleotide; and wherein any additional modified nucleosides are contiguous with either the modified nucleoside positioned at the 5′ end of the second nucleotide or the modified nucleoside positioned at the 3′ end of the second oligonucleotide.

Embodiment 532: The compound of any one of the preceding embodiments, wherein said second oligonucleotide comprises at most four, preferably at most three, more preferably at most two, more preferably one modified nucleoside and wherein said modified nucleoside is 5′ end and/or the 3′ end of said second oligonucleotide.

Embodiment 533: The compound of any one of the preceding embodiments, wherein at least 80% of the internucleoside linkages within said second oligonucleotide are phosphodiester linkages, preferably wherein at least 90% of the internucleoside linkages with said second oligonucleotide are phosphodiester linkages, more preferably wherein all of the internucleoside linkages within said second oligonucleotide are phosphodiester linkages.

Embodiment 534: The compound of any one of the preceding embodiments, wherein at least 70% of the sugar moieties of said second oligonucleotide are unmodified RNA sugar moieties, preferably wherein at least 80%, more preferably at least 90%, more preferably at least 95%, and yet more preferably 100% of the sugar moieties of said second oligonucleotide are unmodified RNA sugar moieties, and wherein preferably at most three of all internucleoside linkages of said second oligonucleotide are phosphorothioate internucleoside linkages, wherein preferably said phosphorothioate internucleoside linkages are positioned at the 5′ end and/or at the 3′ end of said second oligonucleotide.

Embodiment 535: The compound of any one of the preceding embodiments, wherein at least 80%, of the sugar moieties of said second oligonucleotide are unmodified RNA sugar moieties, and the remaining sugar moieties of said second oligonucleotide are abc-DNA sugar moieties. In more preferred embodiments, at least 90% of the sugar moieties of said second oligonucleotide are unmodified RNA sugar moieties, and the remaining sugar moieties of said second oligonucleotide are abc-DNA sugar moieties. In yet more preferred embodiments, at least 95% of the sugar moieties of said second oligonucleotide are unmodified RNA sugar moieties, and the remaining sugar moieties of said second oligonucleotide are abc-DNA sugar moieties.

Embodiment 536: The compound of any one of the preceding embodiments, wherein each sugar moiety of said second oligonucleotide is an unmodified RNA sugar moiety, wherein at most three of all internucleoside linkages of said second oligonucleotide are phosphorothioate internucleoside linkages, wherein preferably said phosphorothioate internucleoside linkages are positioned at the 5′-end and/or at the 3′-end of said second oligonucleotide.

Embodiment 537: The compound of any one of the preceding embodiments, wherein each sugar moiety of said second oligonucleotide is an unmodified RNA sugar moiety, wherein at most two of all internucleoside linkages of said second oligonucleotide are phosphorothioate internucleoside linkages, and wherein preferably said one or two phosphorothioate internucleoside linkages are at the 3′end of said second oligonucleotide.

Embodiment 538: The compound of any one of the preceding embodiments, wherein each sugar moiety of said second oligonucleotide is an unmodified RNA sugar moiety, and wherein each internucleoside linkage of said second oligonucleotide is a phosphodiester linkage.

Embodiment 539: The compound of any one of the preceding embodiments, wherein said 5′ wing of said gapmer comprises up to five unpaired overhanging nucleotides.

Embodiment 540: The compound of any one of the preceding embodiments, wherein said 5′ wing of said gapmer comprises up to four unpaired overhanging nucleotides. In some embodiments, said 5′ wing of said gapmer comprises up to three unpaired overhanging nucleotides. In some embodiments, said 5′ wing of said gapmer comprises up to two unpaired overhanging nucleotides. In some embodiments, said 5′ wing of said gapmer comprises one unpaired overhanging nucleotide.

Embodiment 541: The compound of any one of the preceding embodiments, wherein said 3′ wing of said gapmer comprises up to five unpaired overhanging nucleotides.

Embodiment 542: The compound of any one of the preceding embodiments, wherein said 3′ wing of said gapmer comprises up to four unpaired overhanging nucleotides. In some embodiments, said 3′ wing of said gapmer comprises up to three unpaired overhanging nucleotides. In some embodiments, said 3′ wing of said gapmer comprises up to two unpaired overhanging nucleotides. In some embodiments, said 3′ wing of said gapmer comprises one unpaired overhanging nucleotide.

Embodiment 543: The compound of any one of the preceding embodiments, wherein said gapmer comprises at most ten, preferably at most nine, more preferably at most eight, more preferably at most seven, yet more preferably at most six, yet more preferably at most five overhanging nucleotides.

Embodiment 544: A pharmaceutical composition comprising a compound of any one of the preceding embodiments and a pharmaceutically acceptable carrier.

Embodiment 545: The compound of any embodiments 1-543 or a pharmaceutical composition of embodiment 544, for use in the prevention or treatment of a disease, wherein preferably said disease is a neuromuscular or musculoskeletal disease, and wherein further preferably the neuromuscular or musculoskeletal disease is selected from the group consisting of Duchenne muscular dystrophy, familial dysautonomia, spinal muscular atrophy, ataxia telangiectasia, congenital disorder of glycosylation, fronto-temporal dementia, Parkinsonism linked to chromosome 17, Niemann-Pick disease type C, neurofibromatosis type 1, neurofibromatosis type 2, megalencephalic leukoencephalopathy with subcortical cysts type 1, Pelizaeus-Merzbacher disease, Pompe disease, myotonic dystrophy type 2 (DM2), and myotonic dystrophy type 1 (DM1).

In some preferred embodiments of the invention, Embodiment 467 is combined with any subsequent Embodiment.

In some preferred embodiments of the invention, Embodiment 467 is combined with Embodiment 468.

In some preferred embodiments of the invention, Embodiment 467 is combined with Embodiments 468 and 470.

In some preferred embodiments of the invention, Embodiment 467 is combined with Embodiments 468, 470 and 523.

In some preferred embodiments of the invention, Embodiment 467 is combined with Embodiments 468, 470, 523 and 533.

In some preferred embodiments of the invention, Embodiment 467 is combined with Embodiments 468, 470, 523, 533 and 538.

In some preferred embodiments of the invention, Embodiment 1 is combined with Embodiment 442.

### Method of Treatment

The present invention provides for both prophylactic and therapeutic methods of treating a subject at risk of (or susceptible to) a disease or disorder caused, in whole or in part, by the expression of a target RNA and/or the presence of such target RNA.

“Treatment”, or “treating” as used herein, is defined as the application or administration of a therapeutic agent, in particular of an inventive compound or duplex, to a patient, or application or administration of a therapeutic agent to an isolated tissue or cell line from a patient, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

In one aspect, the invention provides a method for preventing in a subject, a disease or disorder as described above, by administering to the subject a therapeutic agent (e.g., inventive compound or duplex or an inventive pharmaceutical composition).

Another aspect of the invention pertains to methods of treating subjects therapeutically, i.e., alter onset of symptoms of the disease or disorder. These methods can be performed in vitro (e.g., by culturing the cell with the inventive compound or pharmaceutical composition) or, alternatively, in vivo (e.g., by administering the inventive compound or pharmaceutical composition to a subject).

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype”, or “drug response genotype”). Pharmacogenomics allows a clinician or physician to target prophylactic or therapeutic treatments to patients who will most benefit from the treatment and to avoid treatment of patients who will experience toxic drug-related side effects.

Therapeutic agents can be tested in an appropriate animal model. For example, an oligonucleotide agent (or expression vector or transgene encoding same) as described herein can be used in an animal model to determine the efficacy, toxicity, or side effects of treatment with the agent. Alternatively, a therapeutic agent can be used in an animal model to determine the mechanism of action of such an agent. For example, an agent can be used in an animal model to determine the efficacy, toxicity, or side effects of treatment with such an agent. Alternatively, an agent can be used in an animal model to determine the mechanism of action of such an agent.

Moreover, the therapeutic effect of an inventive compound or pharmaceutical composition is determined by assessing muscle function, grip strength, respiratory function, heart function by MRI, muscle physiology. Complement activation and blood coagulation are also determined to investigate the negative side effects of the inventive compound or pharmaceutical composition.

### Diseases

The compounds and pharmaceutical composition of the invention are useful for modulating gene expression by interfering with transcription, translation, splicing and/or degradation and/or by inhibition the function of non-coding RNA, for treatment or prevention of a disease based on aberrant levels of an mRNA or non-coding RNA. A subject is said to be treated for a disease, if following administration, one or more symptoms of the disease are decreased or eliminated.

The compounds and pharmaceutical composition of the invention can modulate the level or activity of a nucleic acid target such as a target RNA. The level or activity of a nucleic acid target can be determined by any suitable method now known in the art or that is later developed. It can be appreciated that the method used to measure a nucleic acid target and/or the expression of a nucleic acid target can depend upon the nature of the nucleic acid target. For example, if the nucleic acid target encodes a protein, the term “expression” can refer to a protein or the RNA/transcript derived from the nucleic acid target. In such instances, the expression of a nucleic acid target can be determined by measuring the amount of RNA corresponding to the nucleic acid target or by measuring the amount of the protein product. Protein can be measured in protein assays such as by staining or immunoblotting or, if the protein catalyzes a reaction that can be measured, by measuring reaction rates. All such methods are known in the art and can be used. Where nucleic acid target levels are to be measured, any art-recognized methods for detecting RNA levels can be used (e.g., RT-PCR, Northern Blotting, etc.). Any of the above measurements can be made on cells, cell extracts, tissues, tissue extracts or any other suitable source material.

The invention provides for treatment or prevention of a disease including but not limited to Duchenne Muscular Dystrophy, Spinal Muscular Atrophy (exon 7 inclusion in the SMN2 gene), Myotonic Dystrophy (target CUGexp-DMPK transcript with CAGₙ), Huntington's disease (allele selective and non-selective approaches targeting the CAG triplet expansion), Amyotrophic lateral sclerosis (genetically heterogeneous disorder with several causative genes), and Pompé disease (target splice mutation c.-32 IVS1-13T>G, which is found in over half of all Caucasian patients.

### Sequences

The sequence of the oligonucleotides, in particular of the first and antisense oligonucleotide can be designed to any target. The sequence of exemplary preferred oligonucleotides, in particular, the preferred sequences of the first oligonucleotide of the invention are provided below.

### DMD Targeting Compounds and Oligonucleotides

Duchenne muscular dystrophy (DMD) affects 1 in 3500 newborn males, while Becker muscular dystrophy (BMD) affects 1 in 20,000. Both DMD and BMD are caused by mutations in the DMD gene, which is located on the X chromosome and codes for dystrophin. DMD patients suffer from progressive muscle weakness, are wheelchair bound before the age of 13, and often die before the third decade of their life. BMD is generally milder and patients often remain ambulant for over 40 years and have longer life expectancies compared to DMD patients.

Dystrophin is an essential component of the dystrophin-glycoprotein complex (DGC). Amongst other things, DGC maintains the membrane stability of muscle fibers. Frame-shifting mutations in the DMD gene result in dystrophin deficiency in muscle cells, which is accompanied by reduced levels of other DGC proteins and results in the severe phenotype found in DMD patients. Mutations in the DMD gene that keep the reading frame intact, generate shorter but partly functional dystrophins, and are associated with the less severe BMD. In Duchnenne Muscular Dystrophy (DMD) patients, frame-shifting mutations in the DMD gene cause an out-of-frame mRNA to be produced, resulting in a truncated, non-functional dystrophin protein. This in-frame mature mRNA encodes an in-frame dystrophin protein that is still partly functional and results in a milder Becker's Muscular Dystrophy (BMD) phenotype.

In certain embodiments the first oligonucleotides of the invention are complementary to portions of the DMD gene, for example, Exon 51, Exon 53 and Exon 45.

### Exon 51

The sequence of exon 51 of the DMD gene (SEQ ID NO:34) is shown below:

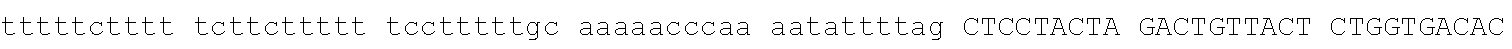

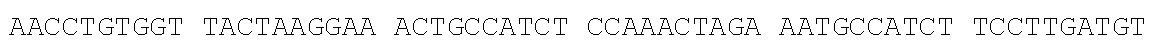

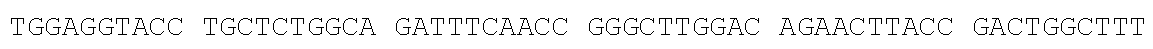

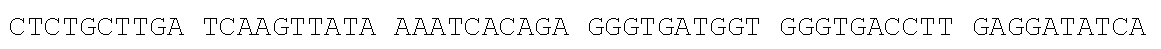

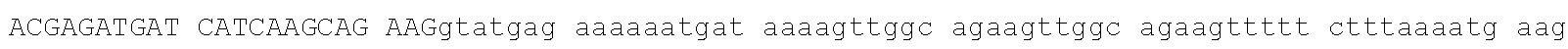

The corresponding transcript sequence of the highlighted portion is:

(SEQ ID NO: 35)
5′ CC AAA CTA GAA ATG CCA TCT TCC TTG ATG T 3′.

First oligonucleotides complementary to Exon 51 of the DMD gene, useful according to the invention have explicitly been described and specifically mentioned in WO 2019/215333 for first oligonucleotides in accordance with present invention, typically and preferably comprising uniformly modified abc-DNA oligonucleotides, the disclosure hereby incorporated by reference in its entirety. Specific reference is made hereby to SEQ ID NOs:1-75, 403 and 404 of WO 2019/215333.

In one embodiment of the present invention, said first oligonucleotide comprises, preferably is, an uniformly modified abc-DNA oligonucleotide, and wherein the sequence of said uniformly modified abc-DNA oligonucleotide is selected from the group consisting of SEQ ID Nos: 36-56.

### Exon 53

The sequence of Exon 53 of the DMD gene (SEQ ID NO: 57) is shown below:

| Exon 53 | | | | | |
|---|---|---|---|---|---|
| 1 | cctccagact | agcatttact | actatatatt | tatttttcct | tttattctag |
| | TTGAAAGAAT | TCAGAATCAG | TGGGATGAAG | TACAAGAACA | CCTTCAGAAC |
| 101 | CGGAGGCAAC | AGTTGAATGA | AATGTTAAAG | GATTCAACAC | AATGGCTGGA |
| | AGCTAAGGAA | GAAGCTGAGC | AGGTCTTAGG | ACAGGCCAGA | GCCAAGCTTG |
| 201 | AGTCATGGAA | GGAGGGTCCC | TATACAGTAG | ATGCAATCCA | AAAGAAAATC |
| | ACAGAAACCA | AGgttagtat | caaagatacc | tttttaaaat | aaaatactgg |
| 301 | ttacatttga | ta | | | |

The corresponding transcript sequence of the highlighted portion is:

(SEQ ID NO: 58)

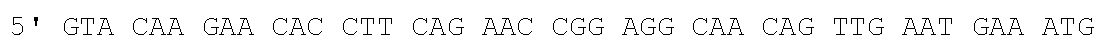

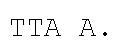

First oligonucleotides complementary to Exon 53 of the DMD gene, useful according to the invention have explicitly been described and specifically mentioned in WO 2019/215333 for first oligonucleotides in accordance with present invention, typically and preferably comprising uniformly modified abc-DNA oligonucleotides, the disclosure hereby incorporated by reference in its entirety. Specific reference is made hereby to SEQ ID NOs:76-240 and 407 of WO 2019/215333.

### Exon 45

The sequence of Exon 45 of the DMD gene (SEQ ID NO: 59) is shown below:

Exon 45

| | | | | | |
|---|---|---|---|---|---|
| 1 | taaaaagaca | tggggcttca | tttttgtttt | gcctttttgg | tatcttacag |
| | GAACTCCAGG | ATGGCATTGG | GCAGCGGCAA | ACTGTTGTCA | GAACATTGAA |
| 101 | TGCAACTGGG | GAAGAAATAA | TTCAGCAATC | CTCAAAAACA | GATGCCAGTA |
| | TTCTACAGGA | AAAATTGGGA | AGCCTGAATC | TGCGGTGGCA | GGAGGTCTGC |
| 201 | AAACAGCTGT | CAGACAGAAA | AAAGAGgtag | ggcgacagat | ctaataggaa |
| | tgaaaacatt | ttagcagact | ttttaa | | |

The corresponding transcript sequence of the highlighted portion is:

(SEQ ID NO: 60)

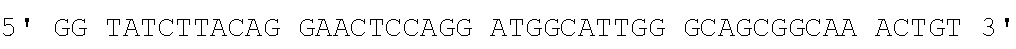

First oligonucleotides complementary to Exon 45 of the DMD gene, useful according to the invention have explicitly been described and specifically mentioned in WO 2019/215333 for first oligonucleotides in accordance with present invention, typically and preferably comprising uniformly modified abc-DNA oligonucleotides, the disclosure hereby incorporated by reference in its entirety. Specific reference is made hereby to SEQ ID NOs:241-400 and 410 of WO 2019/215333.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

All publications, patent applications, patents, and other references mentioned herein are incorporated by reference in their entirety. In case of conflict, the present specification, including definitions, will control.

Although the sequence listing accompanying this application identifies each sequence as either “RNA” or “DNA” as required, in reality, those sequences may be modified with any combination of chemical modifications in accordance with the present invention. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases in accordance with the present invention. By way of further example and without limitation, an oligonucleotide having the nucleobase sequence “ATCGATCG” encompasses any oligonucleotide having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG”, wherein ^{m}C indicates a cytosine base comprising a methyl group at the 5-position.

### EXAMPLES

### Example 1

### Oligonucleotides and Inventive Duplexes

The oligonucleotides listed in Table 3 were synthesized by standard solid phase oligonucleotide synthesis procedures and purified by chromatographic procedures well known in the art. Duplexes in accordance with the present invention were formed by annealing a first and a second single stranded oligonucleotide together to form an inventive duplex.

TABLE 3
List of oligonucleotides

| | SEQ | | | |
|---|---|---|---|---|
| | ID | | | Length |
| Oligo name | NO | Chemistry | Sequence | [nt] |
| abcDNA1 | 1 | abcDNA | 5′-AGCCGAATGGA-7′ | 11 |
| abcDNA2 | 2 | abcDNA | 5′-TTGGAGCCGAATGGA-7′ | 15 |
| abcDNA3 | 3 | abcDNA | 5′-GGTTTGGAGCCGAATGGA-7′ | 18 |
| abcDNA4 | 4 | abcDNA | 5′-TCCATTCGGCTCCAA*Fa-7′ | 15 |
| abcDNA5 | 5 | abcDNA | 5′-TCCATTCGGCTCCAA-7′ | 15 |
| abcDNA6 | 6 | abcDNA | 5′-T*C*C*A*T*T*C*G*G*C*T*C*C*A*A-7′ | 15 |
| PMO1 | 7 | PMO | 5′-AGGTAAGCCGA-3′ | 11 |
| PMO2 | 8 | PMO | 5′-AGGTAAGCCGAGGTT-3′ | 15 |
| PMO3 | 9 | PMO | 5′-AGGTAAGCCGAGGTTTGG-3′ | 18 |
| PMO4 | 10 | PMO | 5′-GGCCAAACCTCGGCTTACCTGAAAT-3′ | 25 |
| MOE1 | 11 | 2′-MOE | 5′-AGGTAAGCCGA-3′ | 11 |
| MOE2 | 12 | 2′-MOE | 5′-AGGTAAGCCGAGGTT-3′ | 15 |
| MOE3 | 13 | 2′-MOE | 5′-AGGTAAGCCGAGGTTTGG-3′ | 18 |
| MOE4 | 14 | 2′-MOE | 5′-C*C*A*A*A*C*C*T*C*G*G*C*T*T*A*C*C* | 18 |
| | | | T-3′ | |
| DNAI | 15 | DNA | 5′-AGGTAAGCCGA-3′ | 11 |
| DNA2 | 16 | DNA | 5′-AGGTAAGCCGAGGTT-3′ | 15 |
| DNA3 | 17 | DNA | 5′-AGGTAAGCCGAGGTTTGG-3′ | 18 |
| FANA1 | 18 | FANA | 5′-AGGTAAGCCGA-3′ | 11 |
| FANA2 | 19 | FANA | 5′-AGGTAAGCCGAGGTT-3′ | 15 |
| FANA3 | 20 | FANA | 5′-AGGTAAGCCGAGGTTTGG-3′ | 18 |
| OMe1 | 21 | 2′-OMe | 5′-AGGTAAGCCGA-3′ | 11 |
| OMe2 | 22 | 2′-OMe | 5′-AGGTAAGCCGAGGTT-3′ | 15 |
| OMe3 | 23 | 2′-OMe | 5′-AGGTAAGCCGAGGTTTGG-3′ | 18 |
| OMe-Pos | 24 | 2′-OMe | 3′-U*C*C*A*U*U*C*G*G*C*U*C*C*A*A*A*C*C* | 20 |
| | | | G*G-5′ | |
| RNA1 | 25 | RNA | 5′-AGGUAAGCCGAGGUU-3′ | 15 |
| RNA2 | 26 | RNA | 5′-AGGUAAGCCGAGGUUUGG-3′ | 18 |
| RNA3 | 27 | RNA | 5′-AUUUCAGGUAAGCCGAGGUUUGGCC-3′ | 25 |
| DNA2-PS1 | 28 | DNA | 5′-AGGTAAGCCGAGGT*T-3′ | 15 |
| DNA2-PS2 | 29 | DNA | 5′-AGGTAAGCCGAGG*T*T-3′ | 15 |
| DNA2-PS3 | 30 | DNA | 5′-A*GGTAAGCCGAGG*T*T-3′ | 15 |
| DNA2-M1 | 31 | DNA/MOE# | 5′-AGGTAAGCCGAGGTt-3′ | 15 |
| DNA2-M2 | 32 | DNA/MOE# | 5′-AGGTAAGCCGAGGtt-3′ | 15 |
| DNA2-M3 | 33 | DNA/MOE# | 5′-aGGTAAGCCGAGGtt-3′ | 15 |
| PMO5 | 61 | PMO | 5′-CTCCAACATCAAGGAAGATGGCATTTCTAG-3′ | 30 |
| FANA4 | 62 | FANA | 5′-CUAGAAAUGCC-3′ | 11 |
| abcDNA-gap1 | 63 | abcDNA/DNA^{$} | 5′-GTCAGCTGCCAAT-3′-7′-gct-5′ | 16 |
| abcDNA-gap2 | 64 | abcDNA/DNA^{$} | 7′-gtc-5′-5′-AGCTGCCAATGCT-3′ | 16 |
| abcDNA-gap3 | 65 | abcDNA/DNA^{$} | 7′-gtc-5′-5′-AGCTGCCAAT-3′-7′-gct-5′ | 16 |
| abcDNA-gap4 | 66 | abcDNA/DNA^{$} | 7′-gggtc-5′-5′AGCTGCCAAT-3′-7′-gctag-5′ | 20 |
| abcDNA-gap5 | 67 | abcDNA/DNA^{$} | 7′-gggtc-5′-5′A*G*C*T*G*C*C*A*A*T-3′- | 20 |
| | | | 7′-gctag-5′ | |
| abcDNA-gap6 | 68 | abcDNA/DNA^{$} | 7′-gggtc-5′-5′AGCTGCCA-3′-7′-atgct-5′ | 18 |
| abcDNA-gap7 | 69 | abcDNA/DNA^{$} | 7′-gggtc-5′-5′AG*C*T*G*C*C*A*AT-3′-7′- | 20 |
| | | | gctag-5′ | |
| abcDNA-pass1 | 70 | abcDNA/RNA^{&} | 7′-ct-5′-5′AGCAUUGGCAGCUGAC-3′-7′-cc-5′ | 20 |
| abcDNA-pass2 | 71 | abcDNA/RNA^{&} | 7′-ctagc-5′-5′-AUUGGCAGCU-3′-7′-gaccc-5′ | 20 |
| MOE-gap | 72 | DNA/MOE^{#} | 5′-g*g*g*t*c*A*G*C*T*G*C*C*A*A*T*g*c*t* | 20 |
| | | | a*g-3′ | |
| DNA-gap1 | 73 | DNA | 5′-GTCAGCTGCCAATGCT-3′ | 16 |
| DNA-gap2 | 74 | DNA | 5′-GGGTCAGCTGCCAATGCTAG-3′ | 20 |
| DNA4 | 75 | DNA | 5′-AGCATTGGCAGCTGAC-3′ | 16 |
| DNA5 | 76 | DNA | 5′-AGCATTGGCAGCTGACCC-3′ | 18 |
| DNA6 | 77 | DNA | 5′-CTAGCATTGGCAGCTGACCC-3′ | 20 |
| RNA4 | 78 | RNA | 5′-AGCAUUGGCAGCUGAC-3′ | 16 |
| RNA5 | 79 | RNA | 5′-AGCAUUGGCAGCUGACCC-3′ | 18 |
| RNA6 | 80 | RNA | 5′-CUAGCAUUGGCAGCUGACCC-3′ | 20 |

* = phosphorothioate linkage; no indication refers to phosphodiester linkage
^{#} = Capital letters refer to DNA, lower case letters refer to MOE
^{$} = Capital letters refer to DNA, lower cases refer to abcDNA
^{&} = Capital letters refer to RNA, lower cases refer to abcDNA
Fa = palmitic acid

### Example 2

### Determination of the Melting Temperature of the Inventive Duplexes

UV melting experiments were recorded on a Varian Cary Bio 100 UV/vis spectrophotometer. Experiments were performed at 2 μM strand concentrations, 10 mM NaH₂PO4, 75 mM NaCl and the pH value was adjusted to 7.0. The samples were protected from evaporation by a covering layer of dimethylpolysiloxane. The absorbance was monitored at λ=260 nm. For every experiment, a cooling-heating cycle was performed with a temperature gradient of 0.5° C./min, run between 20° C. and 90° C. Typically and preferably, the heating and cooling curves are superimposable; if it is not the case, the experiment should be repeated with a lower temperature gradient such as preferably 0.4° C./min or 0.3° C./min. The maxima of the curves first derivative were extracted with the Varian WinUV software and Tm values were reported as the average of the Tm for each curves (Table 4).

TABLE 4
List of Tₘ values in ° C.

| | First Oligonucleotide | | | |
|---|---|---|---|---|
| | | abcDNA4 | MOE4 | PMO4 |
| vs | | SEQ ID NO: 4 | SEQ ID NO: 14 | SEQ ID NO: 10 |
| Second | RNA1 | 74.5 | *** | *** |
| Oligonucleotide | SEQ ID NO: 25 | | | |
| | RNA2 | *** | 83.2 | *** |
| | SEQ ID NO: 26 | | | |
| | RNA3 | *** | *** | 79.9 |
| | SEQ ID NO: 27 | | | |
| | DNA1 | 38.7 | 57.9 | 43.2 |
| | SEQ ID NO: 15 | | | |
| | DNA2 | 52.6 | 60.6 | 59.0 |
| | SEQ ID NO: 16 | | | |
| | DNA3 | 50.0 | 64.0 | 63.7 |
| | SEQ ID NO: 17 | | | |
| | MOE1 | 74.0 | 77.3 | 61.7 |
| | SEQ ID NO: 11 | | | |
| | MOE2 | 83.3 | 81.4 | 83.2 |
| | SEQ ID NO: 12 | | | |
| | MOE3 | 86.9 | 87.0 | 86.9 |
| | SEQ ID NO: 13 | | | |
| | PMO1 | 58.2 | 65.6 | 48.9 |
| | SEQ ID NO: 7 | | | |
| | PMO2 | 71.6 | 81.0 | 61.0 |
| | SEQ ID NO: 8 | | | |
| | PMO3 | 70.9 | 84.5 | 64.4 |
| | SEQ ID NO: 9 | | | |
| | FANA1 | 62.6 | 68.4 | 62.7 |
| | SEQ ID NO: 18 | | | |
| | FANA2 | 74.4 | 79.4 | 71.9 |
| | SEQ ID NO: 19 | | | |
| | FANA3 | 74.2 | 84.6 | 76.7 |
| | SEQ ID NO: 20 | | | |
| | OMe1 | 64.8 | 69.0 | 61.7 |
| | SEQ ID NO: 21 | | | |
| | OMe2 | 80.7 | 86.3 | 76.9 |
| | SEQ ID NO: 22 | | | |
| | OMe3 | 79.3 | 87.5 | 79.3 |
| | SEQ ID NO: 23 | | | |
| | abcDNA1 | 81.8 | 74.5 | 63.5 |
| | SEQ ID NO: 1 | | | |
| | abcDNA2 | 86.7 | 83.0 | 75.3 |
| | SEQ ID NO: 2 | | | |
| | abcDNA3 | 83.1 | 87.3 | 78.1 |
| | SEQ ID NO: 3 | | | |
| | DNA2-PS1 | 49.9 | *** | *** |
| | SEQ ID NO: 28 | | | |
| | DNA2-PS2 | 48.2 | *** | *** |
| | SEQ ID NO: 29 | | | |
| | DNA2-PS3 | 49.0 | *** | *** |
| | SEQ ID NO: 30 | | | |
| | DNA2-M1 | 52.1 | *** | *** |
| | SEQ ID NO: 31 | | | |
| | DNA2-M2 | 50.6 | *** | *** |
| | SEQ ID NO: 32 | | | |
| | DNA2-M3 | 52.6 | *** | *** |
| | SEQ ID NO: 33 | | | |

*** = not measured

### Example 3

### Determination of Biostability of the Inventive Duplexes

The biostability of single strands and inventive duplexes was measured by incubating the samples at a final concentration of 5 μM at 37° C. in mouse serum. The reaction was performed for the following time point: 0 h, 0.5 h, 1 h, 2 h, 4 h, 6 h and 24 h. In addition, the samples were incubated in PBS buffer at 37° C. for 24 h to exclude nuclease presence in the compound preparation. The reactions were stopped at specific times and were analyzed by denaturing AEX-chromatography. Stability was calculated as % full length strand relative to t₀ (Table 5). For each experiment, the experimental values were used to calculate the half-life for the second oligonucleotide as single strand, and for the second oligonucleotide and the first oligonucleotide as part of the inventive duplex, by fitting exponential curves and then extracting the half-life. Such calculations can be performed with Matlab software or with SciPy software by performing a curve fitting using experimental values and the following equation: x=x[0]*exp(−λ*t), where x[0]=percentage of full-length product at time 0 [%]; x=percentage of remaining full-length product at time t [%]; t=time [h]; exp is the exponential function. Fitting the curve to the experimental data set will allow to calculate λ. The half-life can then be calculated by the following formula: t_{1/2}=ln(2)/λ. PMO oligonucleotides could not be resolved by AEX-chromatography, due to their neutral, not charged backbone. However, the high biostability of this chemistry is well established.

TABLE 5
Calculated half-life stability in hours of the first and second oligonucleotide
in inventive duplexes, and of the second oligonucleotide as single strand.

| | | First Oligonucleotide [1] | | | |
|---|---|---|---|---|---|
| | | Single | abcDNA5 | MOE4 | PMO4 |
| vs | | strand | SEQ ID NO: 5 | SEQ ID NO: 14 | SEQ ID NO: 10 |
| Second | DNA3 | 0.17 | | [1] 307.9 | [2] 5.47 |
| Oligonucleotide | SEQ ID NO: 17 | | | [2] 3.59 | |
| [2] | DNA2 | | [1] 227.2 | | |
| | SEQ ID NO: 16 | | [2] 0.80 | | |
| | MOE3 | 30.92 | | [1] 149.2 | [2] 186.02 |
| | SEQ ID NO: 13 | | | [2] 132.06 | |
| | MOE2 | | [1] 213.0 | | |
| | SEQ ID NO: 12 | | [2] 198.60 | | |
| | FANA3 | 0.18 | | [1] 202.1 | [2] 16.55 |
| | SEQ ID NO: 20 | | | [2] 12.46 | |
| | FANA2 | | [1] 219.0 | | |
| | SEQ ID NO: 19 | | [2] 0.45 | | |
| | OMe3 | 2.59 | | [1] 299.6 | [2] 58.32 |
| | SEQ ID NO: 23 | | | [2] 170.76 | |
| | OMe2 | | [1] 239.6 | | |
| | SEQ ID NO: 22 | | [2] 5.15 | | |
| | abcDNA3 | 326.62 | | [1] 371.2 | [2] 415.73 |
| | SEQ ID NO: 3 | | | [2] 349.09 | |
| | abcDNA2 | | [1] 134.6 | | |
| | SEQ ID NO: 2 | | [2] 156.49 | | |
| | DNA2-PS1 | 2.32 | [1] 147.8 | | |
| | SEQ ID NO: 28 | | [2] 3.23 | | |
| | DNA2-PS2 | 1.95 | [1] 203.7 | | |
| | SEQ ID NO: 29 | | [2] 1.96 | | |
| | DNA2-PS3 | 1.89 | [1] 436.8 | | |
| | SEQ ID NO: 30 | | [2] 1.98 | | |
| | DNA2-M1 | 0.47 | [1] 361.6 | | |
| | SEQ ID NO: 31 | | [2] 4.05 | | |
| | DNA2-M2 | 3.51 | [1] 291.2 | | |
| | SEQ ID NO: 32 | | [2] 9.26 | | |
| | DNA2-M3 | 2.75 | [1] 246.9 | | |
| | SEQ ID NO: 33 | | [2] 9.89 | | |

### Example 4

### In Vitro Transfection and In Vitro Gymnotic Experiments Using ss-AONs and Inventive Duplexes Targeting Exon 23 of Dystrophin Pre-mRNA—Cell Culture and Skipping Analyses

Experiments were conducted in mice control immortalized myoblast cultures (C2C12). The cells were propagated and differentiated into myotubes using standard culturing techniques. The cells were treated with the single stranded AONs as well as with inventive duplexes by using a transfection reagent or by naked delivery (gymnosis). Each experiment was performed at least in duplicate. Complementary AON with a 2′-OMe-phosphorodithioate (PS-2′OMe) (OMe-Pos; SEQ ID NO:24) backbone and a scrambled (non-functional) PS-2′OMe AON were used as positive and negative controls, respectively.

After treatment with the single stranded AONs and inventive duplexes, respectively, total RNA was extracted, and molecular analysis was conducted. Reverse transcriptase amplification (RT-PCR), using a two-step (nested) PCR reaction, was undertaken to study the targeted regions of the dystrophin pre-mRNA or induced exonic rearrangements.

For analyzing the efficacy of the used single stranded AONs and inventive duplexes, respectively, to induce skipping of exon 23 and of exon 22+23, the RT-PCR was conducted on the region spanning exon 22 and 23. After cDNA synthesis, first round PCR was performed using specific primers in mouse exons 20 and 26 (region 20-26) and the second round PCR was performed using specific primers in mouse exons 21 and 24 (region 21-24). The reactions were analyzed on an agarose gel, including a size standard. Skipping efficacy was quantified with an image processing program (ImageJ).

Single stranded AONs, namely PO-abcDNA (abcDNA4, SEQ ID NO:4) and PS-abcDNA (abcDNA6, SEQ ID NO:6) were selected as reference for in vitro experiments, as this biostable chemistry (abcDNA) shows good efficacy by transfection but low efficacy by gymnosis. For improved interaction with the transfecting reagent, the PO-abcDNA was covalently linked to a fatty acid leading to abcDNA4 (SEQ ID NO:4) and was used as such for the assays.

### In Vitro Transfection Experiments

For the formation of duplexes in accordance with the present invention, the two strands were mixed in PBS in 1:1 ratio, the resulting solutions were heated at 90° C. for 3 min and then cooled down at room temperature for 45 minutes. The cells were transfected with 3 μg of the AONs applied as single stranded or as inventive duplexes, respectively, by using Lipofectamine 2000 as a transfection reagent. Cell were harvested after 24 h treatment and mRNA skipping efficacy was analyzed as indicated previously.

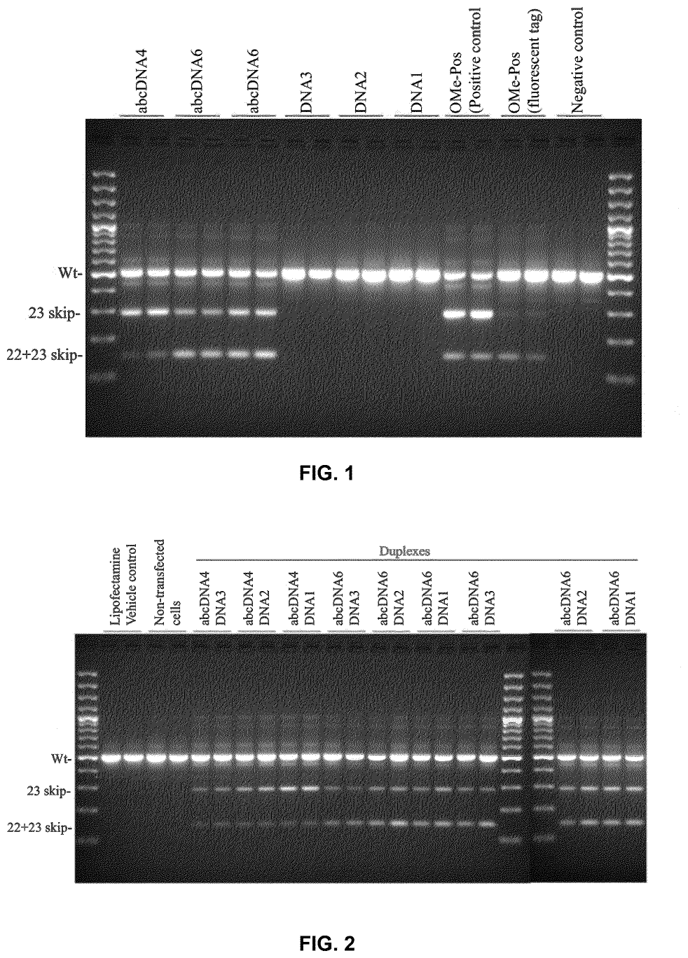

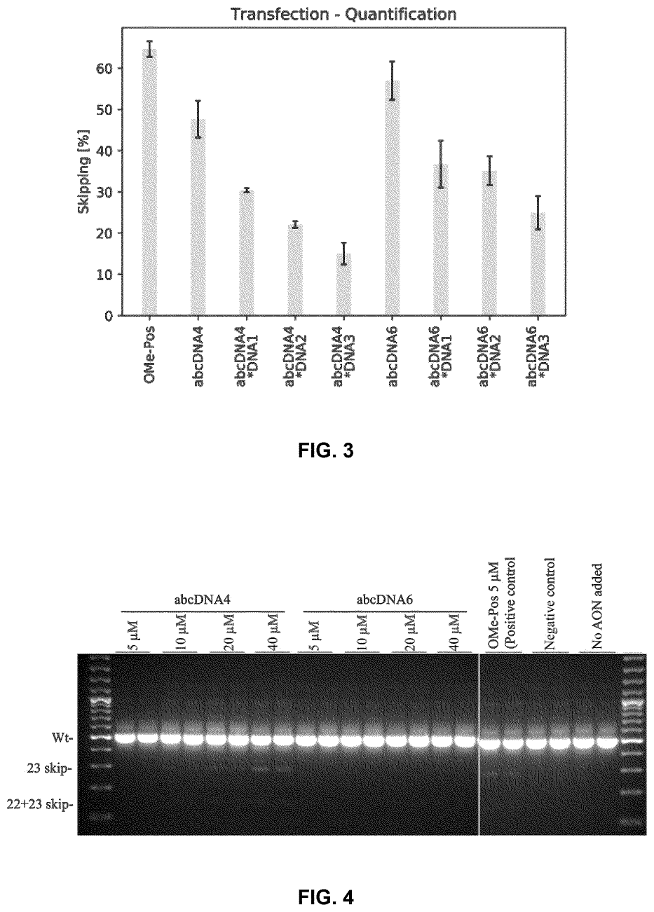

Transfection experiments were performed with single-stranded (ss-)AONs (FIG. 1) and with double-stranded (ds-)AONs, and thus with duplexes in accordance with the present invention (FIG. 2). The relevant skipping activities were quantified with an image processing program (FIG. 3). All single stranded AONs (abcDNA4, abcDNA6 and OMe-Pos) were efficiently able to produce the expected skipped products. On the other hand, the complementary DNA sequences (DNA1, DNA2 and DNA3) did not render any skipped products, testifying for the inactivity of the cDNAs alone. While transfected, the respective AONs applied as duplexes in accordance with the present invention showed reduced efficacies. This data indicates that, once delivered into the cytoplasm via transfection, the formation of duplexes does not enhance the AONs efficacy. On the opposite, the cDNAs act as competitive inhibitors, as expected. The observed increased activity in case of the in vitro gymnotic experiments (see below) is thus believed not to be due to higher efficacy in the cytoplasm, but due to higher cellular delivery.

### In Vitro Gymnotic Experiments

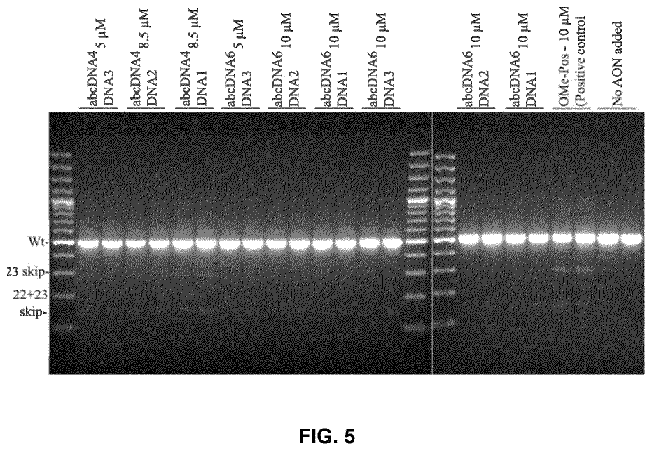

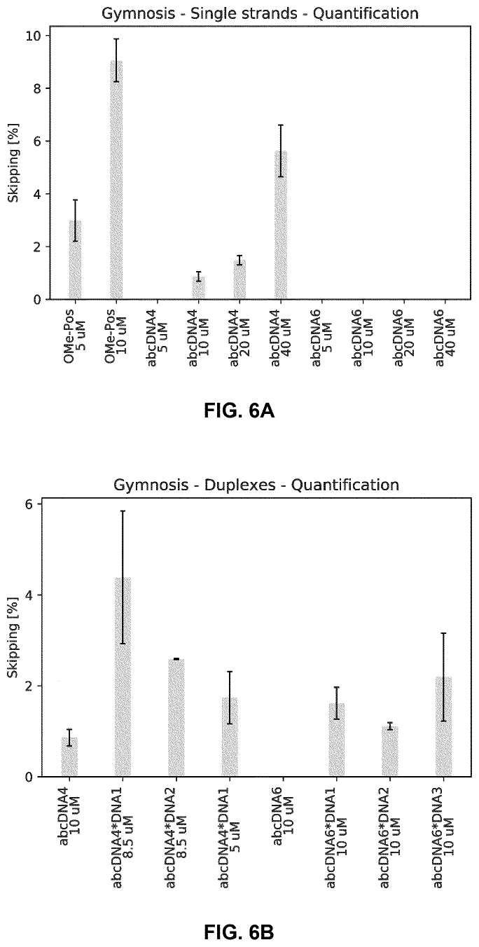

For the formation of duplexes in accordance with the present invention, the two strands were mixed in PBS in 1:1 ratio, the resulting solutions were heated at 90° C. for 3 min and then cooled down at room temperature for 45 minutes. The cells were treated with the single stranded AONs and inventive duplexes, respectively, at different concentration without any delivery reagent. Cells were harvested after 74 h treatment and mRNA skipping efficacy was analyzed as indicated previously. Gymnotic experiments were performed for ss-AONs at concentrations increasing from 5 to 40 μM (FIG. 4) and with ds-AONs and thus with duplexes in accordance with the present invention (FIG. 5). The relevant skipping activities were quantified with an image processing program (FIG. 6). The gymnotic experiments with ds-abcDNA compounds were performed in concentration ranging from 5 to 10 μM.

When the cells were treated with ss-AONs, the PO-abcDNA (abcDNA4, SEQ ID NO:4) induced detectable but weak exon skipping at 10 and 20 μM (<2%), and relevant exon sipping at 40 μM (5.6%). The PS-abcDNA (abcDNA6, SEQ ID NO:6) was not able to induce detectable exon skipping even at 40 μM.

On the other hand, when the cells were treated with abcDNA AONs as inventive duplexes, a significant increase in skipping efficacy was detected for all experiments. For the inventive duplexes applying the PO-abcDNA as AON, the skipping efficacy increased from 3- to 8-fold when compared to the corresponding ss-AON at 10 μM, and thus despite being used at lower concentrations. More drastically, the PS-abcDNA AONs formulated as duplexes were able to induce detectable exon skipping at 10 μM, while the corresponding ss-AON was not able to render any skipping activity even at 40 μM. Taken together, this data clearly indicates that the duplexes in accordance with the present invention significantly improves the cellular delivery and the efficacy of AONs in vitro.

### Example 5

### In Vitro Intra-Muscular Injections Using Single Strand AONs and Inventive Duplexes Targeting Exon 23 of Dystrophin Pre-mRNA

The animal experiments were carried out at the animal facility of the Leiden University Medical Center following the guidelines of and were approved by the Animal Ethics Committee (DEC) of the Leiden University Medical Center. Nine- to thirteen weeks old male mdx mice were anesthetized and then injected intra-muscularly in the gastrocnemius right (GR) and left (GF) and the triceps right (TR) and left (TF) with single stranded AONs, namely abcDNA4 (SEQ ID NO:4), abcDNA6 (SEQ ID NO:6) and OMe-Pos (SEQ ID NO:24), as well as with duplexes in accordance with the present invention, namely abcDNA4 in duplex with DNA1 (SEQ ID NO:15) or DNA3 (SEQ ID NO:17). The single stranded AONs and inventive duplexes, respectively, were injected 2 times over 2 consecutive days, at an injection dose of 50 μg (relative to the ss-AON) dissolved in 40 μL saline solution. Ten days after the last injection mice were killed by cervical dislocation and muscles tissues were snap-frozen in dry ice-cooled iso-pentane and stored at −80° C.

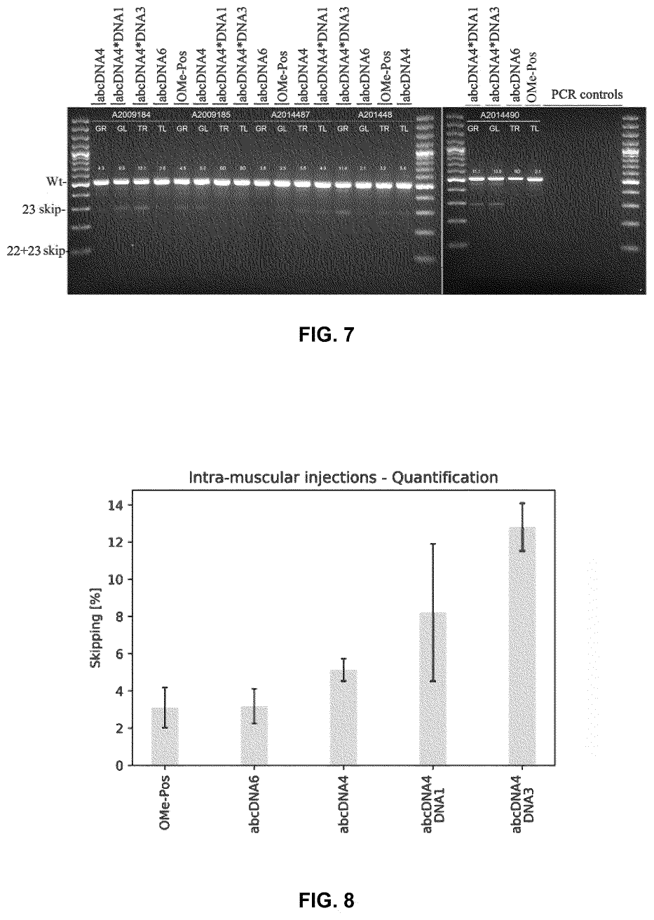

Total RNA was extracted from tested muscle tissues with Trizol reagent as per manufacturer's protocol. cDNA synthesis was performed with 400 ng RNA and then analysis was performed by RT-PCR following the protocol described in Example 4. The reactions were analyzed on an agarose gel, including a size standard (FIG. 7). In addition, precise quantification of the skipped product was performed with a Lab-on-a-chip (FIG. 8). Three outliner samples having skipped products below detect limit (BD) were excluded for the quantification.

The three ss-AONs (OMe-Pos, abcDNA6 and abcDNA4) display a relatively similar exon skipping activity (3.10%, 3.16% and 5.13% respectively), with PO-abcDNA (SEQ ID NO:4) being the most active compound. When injected as a duplex in accordance with the present invention, the exon skipping activity of PO-abcDNA increased to 8.21% with its 11-mer DNA complement (DNA1) and to 12.80% with its 15-mer DNA complement (DNA3). In the former case, this represent a statistically significant 2.5-fold increase in activity. In line with the cellular experiment, this data clearly indicates that the duplexes in accordance with the present invention significantly improves the cellular delivery and the efficacy of AONs in vivo as well.

### Example 6

### Additional In Vitro Gymnotic Experiments Using ss-AONs and Inventive Duplexes Targeting Dystrophin Pre-mRNA

Experiments were conducted in mice control immortalized myoblast cultures (C2C12) for abcDNA4 (SEQ ID NO: 4) and MOE4 (SEQ ID NO: 14) and control immortalized human myoblast cultures (KM155) for PMO5 (SEQ ID NO: 34). The cells were propagated and differentiated into myotubes using standard culturing techniques. The cells were treated with the AONs by naked delivery (gymnosis). Each experiment was performed at least in duplicate. Complementary AONs with a 2′-OMe-phosphorodithioate (PS-2′OMe) backbone and a scrambled (non-functional) PS-2′OMe AON were used as positive (OMe-Pos) and negative controls, respectively.

For the formation of duplexes, the two strands were mixed in PBS in 1:1 ratio, and the resulting solution was heated at 90° C. for 3 min and then cooled to rt for 45 minutes. The cells were treated with AONs at 10 μM concentration without any delivery reagent. Cells were harvested after 74 h treatment, total RNA was extracted and molecular analysis was conducted. Reverse transcriptase amplification (RT-PCR), using a two-step (nested) PCR reaction, was undertaken to study the targeted regions of the dystrophin pre-mRNA or induced exonic rearrangements. For C2C12 cells, RT-PCR was performed as indicated in Example 4. For KM155 cells, after cDNA synthesis, first round PCR was performed using specific primers in human exons 48 and 53 and the second round PCR was performed using specific primers in human exons 49 and 52. Skipping efficacy was quantified by a “lab on a chip.”

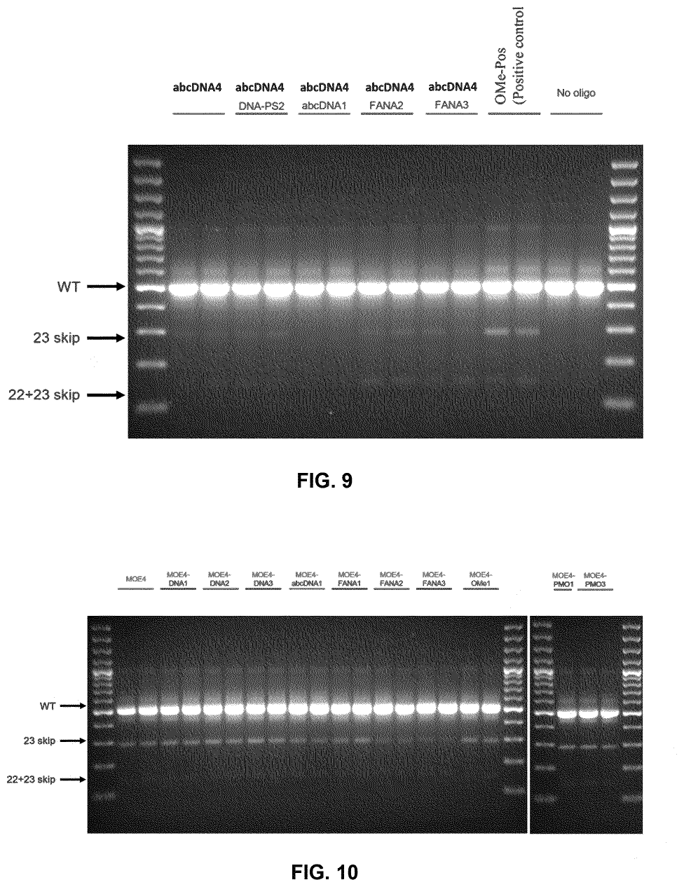

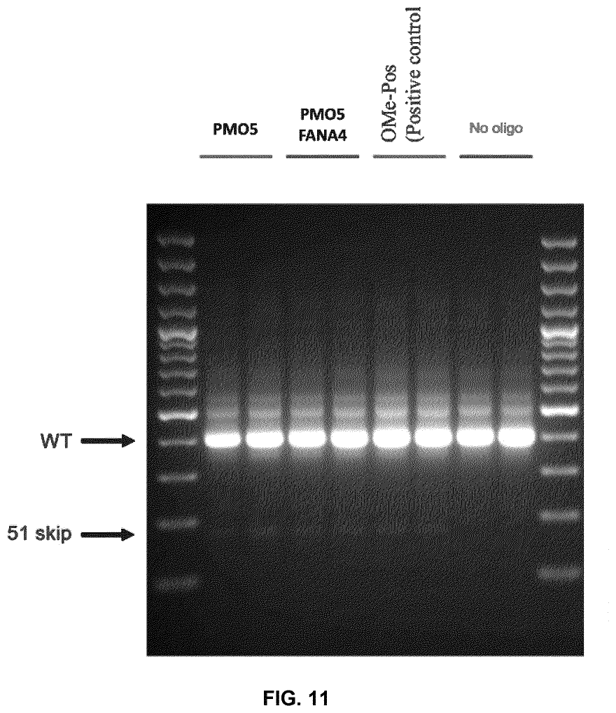

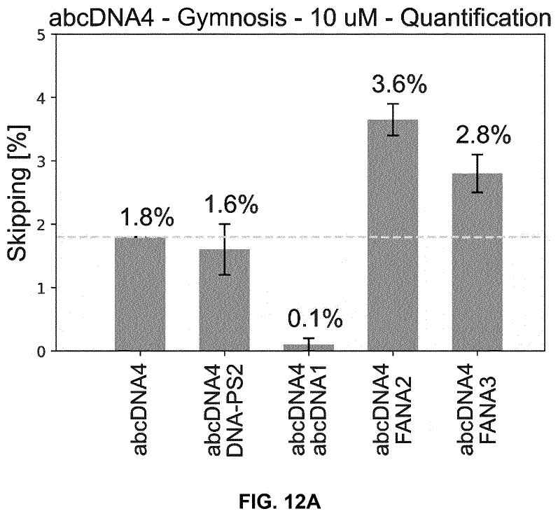

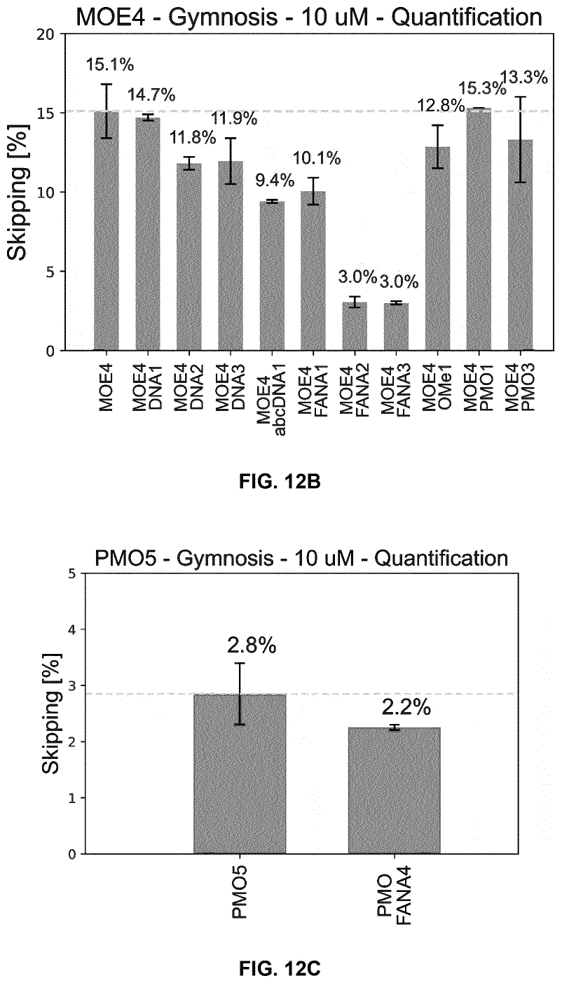

For abcDNA4 (SEQ ID NO: 4) (FIG. 9 and FIG. 12A), the single strand resulted in weak skipping. However, when abcDNA4 was preformulated as inventive duplex with FANA1 (SEQ ID NO: 18) or FANA2 (SEQ ID NO: 19) the activity increased up to 2-fold. For MOE4 (SEQ ID NO: 14) (FIG. 10 and FIG. 12B), the single strand showed robust skipping activity, but when preformulated as duplex, the activity decreased. Similarly, PMO5 (SEQ ID NO: 34) resulted in weak skipping as single strand and slightly decreased skipping when preformulated as duplex (FIG. 11 and FIG. 12C).

### Example 7

### Additional In Vivo Intra-Muscular Injections Using Single Strand AONs and Inventive Duplexes Targeting Exon 23 of Dystrophin Pre-mRNA

The animal experiments were carried out at the animal facility of the Leiden University Medical Center following the guidelines of and were approved by the Animal Ethics Committee (DEC) of the Leiden University Medical Center. Eight- to nine-week old male mdx mice were anesthetized and then injected intra-muscularly in the gastrocnemius right (GR) and left (GF) and the triceps right (TR) and left (TF) with AONs either as single strand (n=6) or as duplexes (n=3). Each formulation was injected 2 times over 2 consecutive days, at an injection dose of 50 μg (relative to the ss-AON) dissolved in 40 μL saline solution. 2 weeks after the last injection mice were killed by cervical dislocation and muscle tissues were snap-frozen in dry ice-cooled isopentane and stored at −80° C.

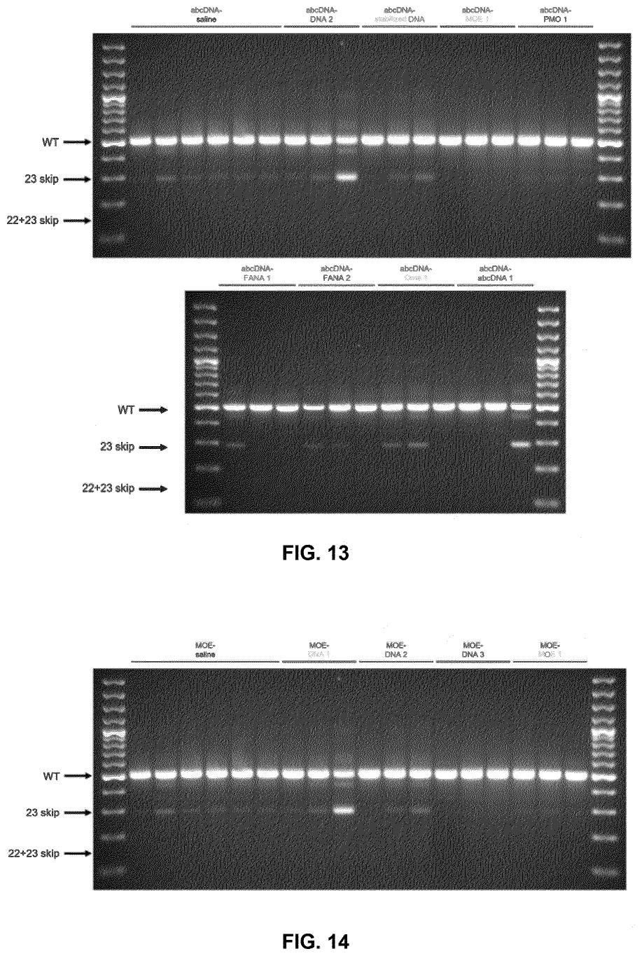

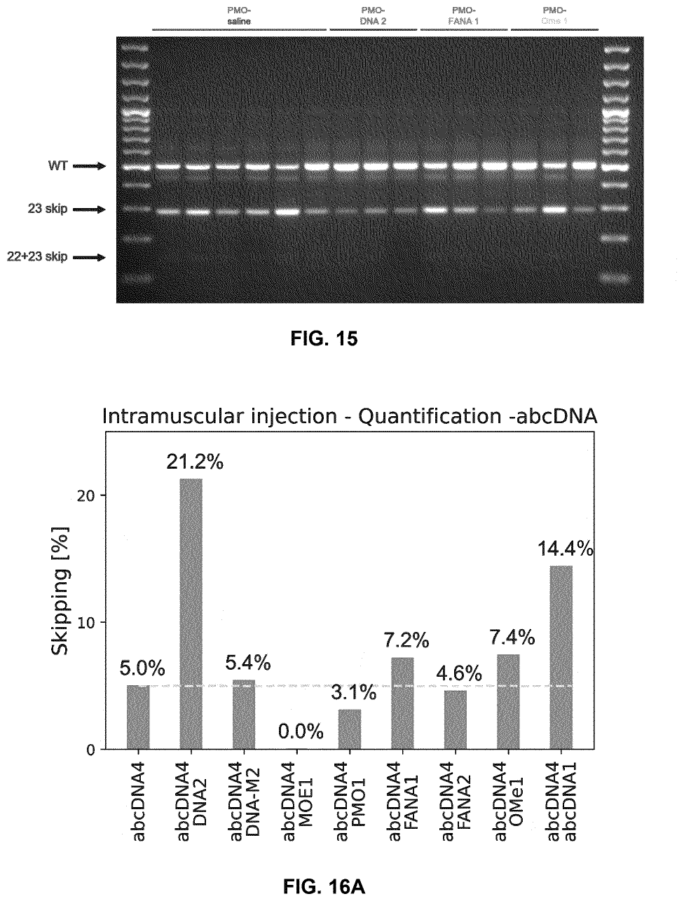

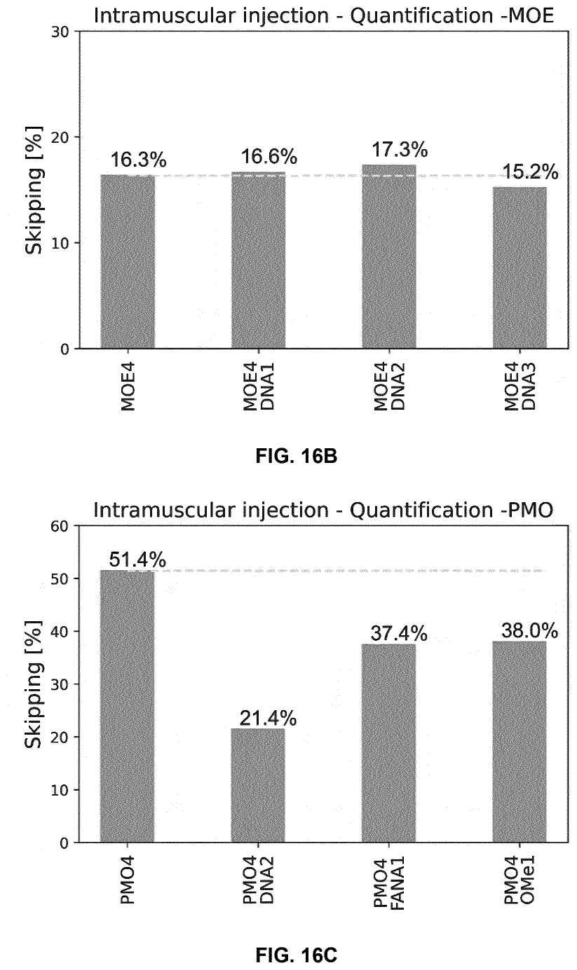

Total RNA was extracted from tested muscle tissues with Trizol reagent as per manufacturer's protocol. cDNA synthesis was performed with 400 ng RNA and then analysis was performed by RT-PCR following the protocol described in Example 4. The reactions were analyzed on an agarose gel, including a size standard (FIG. 13, FIG. 14, and FIG. 15). In addition, precise quantification of the skipped product was performed with a Lab-on-a-chip (FIG. 16).

For abcDNA4 (SEQ ID NO: 4), preformulating the AON as an inventive duplex with complementary DNA2 (SEQ ID NO: 16), FANA1 (SEQ ID NO: 18), OMe1 (SEQ ID NO: 21) or abcDNA1 (SEQ ID NO: 1) increased the efficacy when compared to the corresponding single strand (FIG. 13 and FIG. 16A). For MOE4 (SEQ ID NO: 14), preformulating the AON as a duplex with complementary oligonucleotides resulted in limited skipping efficacy when compared to the corresponding single strand (FIG. 14 and FIG. 16B). For PMO4 (SEQ ID NO: 10), preformulating the AON as duplex did not increase the efficacy when compared to the corresponding single strand (FIG. 15 and FIG. 16C).

The increase in skipping efficacy exhibited by the inventive duplexes comprising abcDNA4 (SEQ ID NO: 4) and DNA2 (SEQ ID NO: 16) compared to the corresponding single strand was confirmed using six additional mice according to the protocol described above. The increase in skipping efficacy of the inventive duplexes when tested in six additional mice was 10.3%.

### Example 8

### Determination of the Melting Temperature of Gapmers and Inventive Duplexes

UV-melting experiments were recorded at 2 μM strands concentration, in 10 mM NaH₂PO₄, 150 mM NaCl and pH adjusted to 7.0. Absorbance was monitored at 260 nm. For every experiment, two cooling-heating cycles were performed with a temperature gradient of 0.5° C./min, between 20° C. and 90° C. The melting temperatures were calculated using the first derivative of the melting curve and Tₘ values were reported as the average.

TABLE 6
List of Tₘ values in ° C.

| Oligo ID strand 1 | Oligo ID strand 2 | Tₘ [° C.] |
|---|---|---|
| abcDNA-gap1 (SEQ ID NO: 63) | DNA4 (SEQ ID NO: 75) | 61.7 |
| | RNA4 (SEQ ID NO: 78) | 64.7 |
| abcDNA-gap2 (SEQ ID NO: 64) | DNA4 (SEQ ID NO: 75) | 62.6 |
| | RNA4 (SEQ ID NO: 78) | 63.0 |
| abcDNA-gap3 (SEQ ID NO: 65) | DNA4 (SEQ ID NO: 75) | 58.1 |
| | RNA4 (SEQ ID NO: 78) | 61.8 |
| abcDNA-gap4 (SEQ ID NO: 66) | DNA6 (SEQ ID NO: 77) | 66.4 |
| | RNA6 (SEQ ID NO: 80) | 71.5 |
| | abcDNA-pass1 (SEQ ID NO: 70) | 73.1 |
| | abcDNA-pass2 (SEQ ID NO: 71) | 76.3 |
| abcDNA-gap5 (SEQ ID NO: 67) | DNA6 (SEQ ID NO: 77) | 57.7 |
| | RNA6 (SEQ ID NO: 80) | 68.1 |
| abcDNA-gap6 (SEQ ID NO: 68) | DNA5 (SEQ ID NO: 76) | 63.6 |
| | RNA5 (SEQ ID NO: 78) | 70.6 |
| abcDNA-gap7 (SEQ ID NO: 69) | DNA6 (SEQ ID NO: 77) | 62.1 |
| | RNA6 (SEQ ID NO: 80) | 68.8 |
| MOE-gap (SEQ ID NO: 72) | DNA6 (SEQ ID NO: 77) | 64.8 |
| | RNA6 (SEQ ID NO: 80) | 72.8 |
| abcDNA-pass1 (SEQ ID NO: 70) | DNA-gap2 (SEQ ID NO: 74) | 68.3 |
| abcDNA-pass2 (SEQ ID NO: 71) | DNA-gap2 (SEQ ID NO: 74) | 68.3 |

### Example 9

### In Vitro Activity Based on a RNase H Assay

The gapmers (DNA-gap1 (SEQ ID NO: 73), abcDNA-gap1 (SEQ ID NO: 63), abcDNA-gap2 (SEQ ID NO: 64), abcDNA-gap3 (SEQ ID NO: 65), DNA-gap2 (SEQ ID NO: 74), abcDNA-gap4 (SEQ ID NO: 66), abcDNA-gap5 (SEQ ID NO: 67) and MOE-gap (SEQ ID NO: 72)) and complementary RNA4 (SEQ ID NO: 78) or RNA6 (SEQ ID NO: 80) were diluted at a concentration of 0.05:0.1; 0.1:0.2; 0.25:0.5; 0.5:1.0; and 1.0:2.0 μM (AON:RNA) in 192 μL of a solution of 20 mM Tris-HCl (pH 7.8), 40 mM KCl, 8 mM MgCl₂, (10× solution: 200 mM Tris-HCl (pH 7.8), 400 mM KCl, 80 mM MgCl₂). The mixtures were then transferred to two cuvettes (96 μL each) (BRAND UV cuvette micro, Sigma), one serving as a blank and the other serving as the reaction cuvette. The mixtures were then heated at 37° C. for 30 min. The absorbance was recorded at 260 nm.

A RNase H solution (0.5 U/μL) was prepared by dissolving a 10× solution of RNase H (Thermofisher, RNase H (5 U/μL), Catalog number: EN0201, source: E. coli) in a solution of 20 mM Tris-HCl (pH 7.8), 40 mM KCl, 8 mM MgCl₂.

The reaction was initiated by adding 4.0 μL of a solution of 20 mM Tris-HCl (pH 7.8), 40 mM KCl, 8 mM MgCl₂ to the blank cuvette and by adding 4.0 μL of the RNase H solution to the reaction cuvette. The mixtures were then protected from evaporation by a thin layer of polydimethylsiloxane.

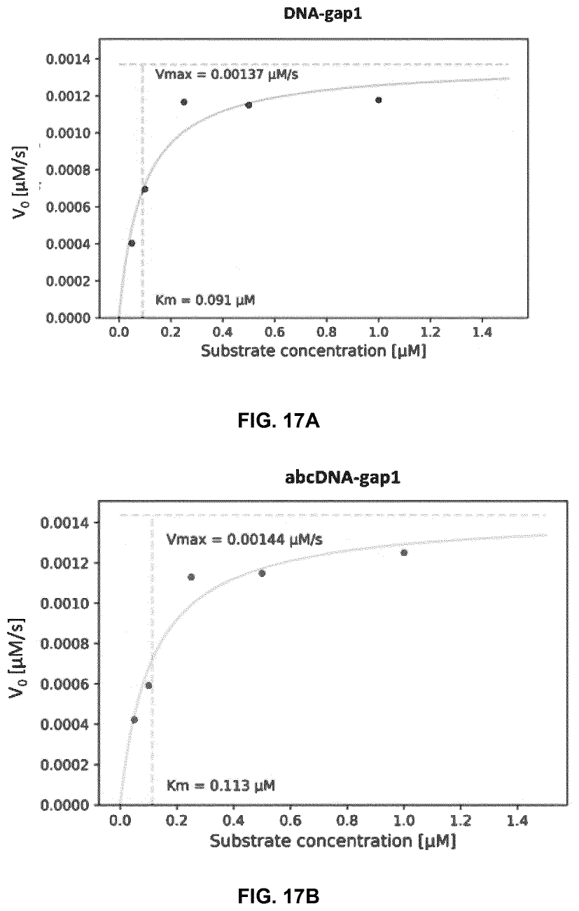

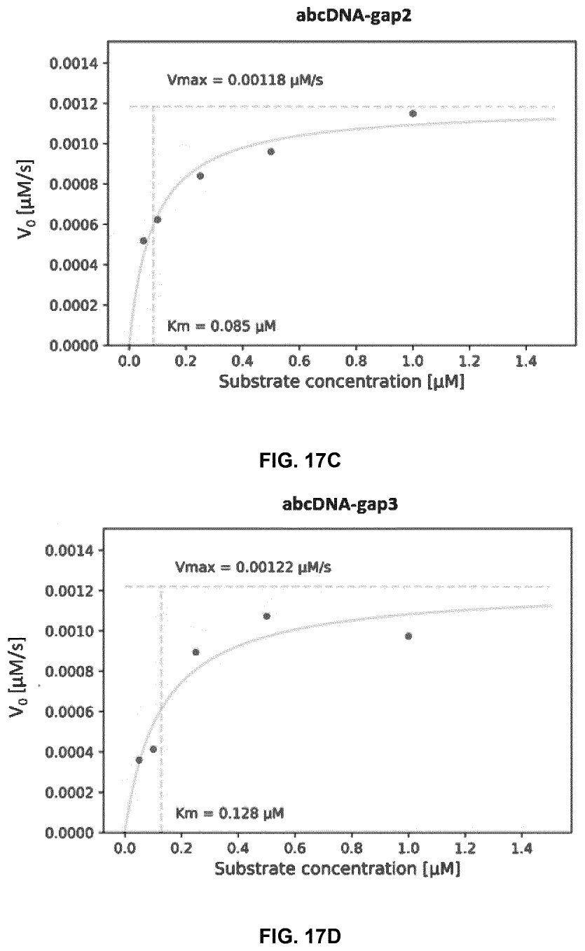

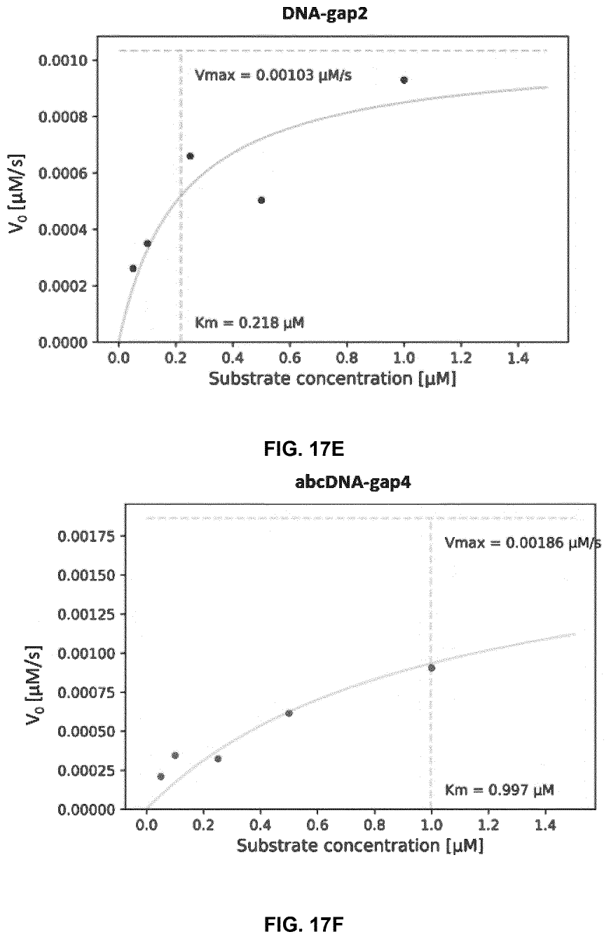

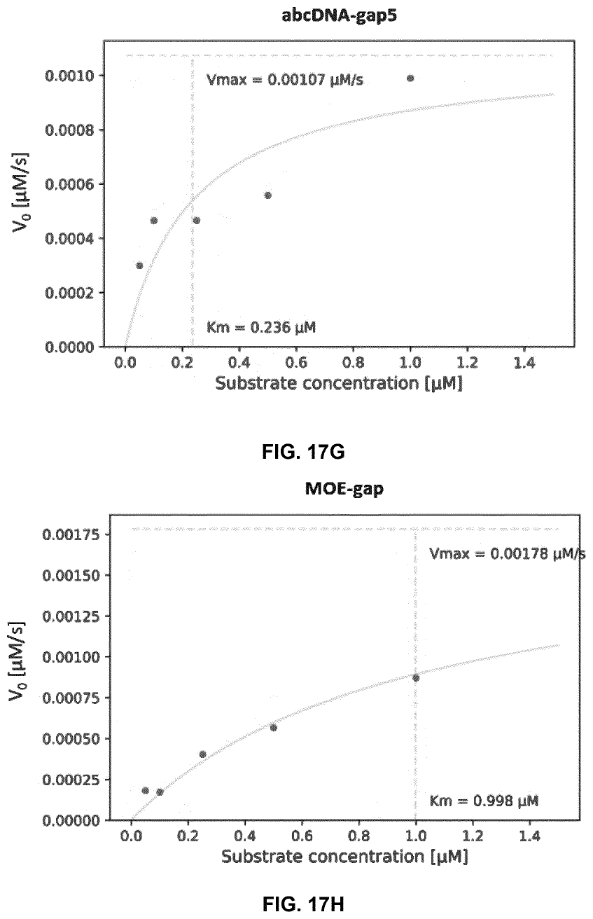

The reaction outcomes were monitored by recording the absorbance at 260 nm. For analyses, the absorbances at time 0 were subtracted from other timepoints. The absorbance at time 0 was set as 0% product formation, and the absorbance at time final (final time=10 h) was set as 100% product formation. Thus, the reaction advancement was calculated based on the change of absorbance. The initial speed of reaction was calculated, depicted in FIGS. 17A-17H, and used to calculate the kinetic parameters (Vₘₐₓ and Kₘ) also reported in FIGS. 17A-17H.

### Example 10

### Determination of Biostability of Gapmers and Inventive Duplexes Thereof

The biostability of single strands and inventive duplexes was measured by incubating the samples at a final concentration of 20 μM at 37° C. in mouse serum. The reaction was performed for the following time points: 0 h, 0.5 h, 1 h, 2 h, 5 h and 24 h. The reactions were stopped at specific times and were analyzed by ion-pair reversed-phase HPLC. Stability was calculated as percent full length strand relative to t₀ (Table 7). For each experiment, the experimental values were used to calculate the half-life for the first oligonucleotide as single strand, and for the first oligonucleotide as part of the inventive duplex, by fitting exponential curves and then extracting the half-life. Calculations were performed with SciPy Python software by performing a curve fitting using experimental values and the following equation:

x=x[0]^{(-λt)} (Equation 1)

where x[0]=percentage of full-length product at time 0 [%]; x=percentage of remaining full-length product at time t [%]; t=time [h]. Fitting the curve to the experimental data set allows one to calculate λ. The half-life was then calculated by the following formula:

t_{1/2}=ln(2)/λ.

TABLE 7
Calculated half-life stability in hours of the first oligonucleotide
as single strand or in inventive duplexes.

| | First Oligonucleotide [1] (gapmer) | | | | | | |
|---|---|---|---|---|---|---|---|
| | | DNA- | abcDNA- | abcDNA- | abcDN- | abcDNA- | MOE- |
| | | gap2 | gap4 | gap5 | gap6 | gap7 | gap |
| | | (SEQ ID | (SEQ ID | (SEQ ID | (SEQ ID | (SEQ ID | (SEQ ID |
| vs | | NO: 74) | NO: 66) | NO: 67) | NO: 68) | NO: 69) | NO: 72) |
| Second | Single | 0.34 | 1.79 | 58.94 | 1.71 | 2.29 | 1.22 |
| Oligonucleotide | strand | | | | | | |
| [2] | DNA6 | n.d. | 0.20 | n.d. | n.d. | 2.44 | n.d. |
| | (SEQ ID | | | | | | |
| | NO: 77) | | | | | | |
| | DNA5 | n.d. | n.d. | n.d. | 0.29 | n.d. | n.d. |
| | (SEQ ID | | | | | | |
| | NO: 76) | | | | | | |
| | RNA6 | n.d. | 3.57 | n.d. | n.d. | n.d. | n.d. |
| | (SEQ ID | | | | | | |
| | NO: 80) | | | | | | |
| | abcDNA- | n.d. | 5.16 | n.d. | n.d. | n.d. | n.d. |
| | pass1 | | | | | | |
| | (SEQ ID | | | | | | |
| | NO: 70) | | | | | | |
| | abcDNA- | n.d. | 1.25 | n.d. | n.d. | n.d. | n.d. |
| | pass2 | | | | | | |
| | (SEQ ID | | | | | | |
| | NO: 71) | | | | | | |

1. A compound comprising a first oligomeric compound and a second oligomeric compound, wherein the first oligomeric compound comprises a first oligonucleotide and said second oligomeric compound comprises a second oligonucleotide, wherein
said first oligonucleotide comprises at least one abc-DNA nucleoside, wherein said first oligonucleotide has a nucleobase sequence that is complementary to a nucleic acid target, and wherein preferably said first oligonucleotide is an antisense oligonucleotide; and wherein
said second oligonucleotide has a nucleobase sequence that is complementary to the nucleobase sequence of the first oligonucleotide.

2. The compound of claim 1, wherein at least 50% of the nucleosides of the first oligonucleotide are abc-DNA nucleosides.

3. The compound of claim 1 or claim 2, wherein said first oligonucleotide comprises at least 8 and at most 50 nucleotides.

4. The compound of any one of the preceding claims, wherein at most five, preferably at most four, further preferably at most three, more preferably at most two, and yet more preferably only one of all internucleoside linkages of said first oligonucleotide are phosphorothioate internucleoside linkages, and the remaining internucleoside linkages of said first oligonucleotide are phosphodiester linkages.

5. The compound of any one of the preceding claims, wherein all internucleoside linkages of said first oligonucleotide are phosphodiester linkages.

6. The compound of any one of the preceding claims, wherein at least 70% of the sugar moieties of said second oligonucleotide are unmodified DNA sugar moieties, preferably wherein at least 80%, more preferably at least 90%, more preferably at least 95%, and yet more preferably 100% of the sugar moieties of said second oligonucleotide are unmodified DNA sugar moieties, and wherein preferably at most three of all internucleoside linkages of said second oligonucleotide are phosphorothioate internucleoside linkages, wherein preferably said phosphorothioate internucleoside linkages are positioned at the 5′-end and/or at the 3′-end of said second oligonucleotide.

7. The compound of any one of the preceding claims, wherein each sugar moiety of said second oligonucleotide is an unmodified DNA sugar moiety, wherein at most four of all internucleoside linkages of said second oligonucleotide are phosphorothioate internucleoside linkages, wherein preferably said phosphorothioate internucleoside linkages are positioned at the 5′-end and/or at the 3′-end of said second oligonucleotide.

8. The compound of any one of the claims 1 to 6, wherein said second oligonucleotide comprises at least one modified sugar moiety selected from the group consisting of (i) a non-bicyclic modified sugar moiety comprising a furanosyl ring with one or more acyclic substituent at the 2′, 4′, and/or 5′ position, (ii) a bicyclic sugar moiety, and (iii) a sugar surrogate.

9. The compound of any one of the claims 1 to 6 and 8, wherein said second oligonucleotide consists of modified nucleosides selected from the group consisting of (i) a non-bicyclic modified sugar moiety comprising a furanosyl ring with one or more acyclic substituent at the 2′, 4′, and/or 5′ position, (ii) a bicyclic sugar moiety, (iii) a sugar surrogate.

10. The compound of any one of the claims 1 to 6 and 8 to 9, wherein said second oligonucleotide consists of modified nucleosides, wherein said modified nucleosides are selected from the group consisting of a 2′-MOE nucleoside, 2′-OMe nucleoside, an abc-DNA nucleoside, a FANA, and a morpholino.

11. The compound of any one of the claims 1 to 3, wherein said first oligonucleotide is a gapmer comprising a gap, a 5′ wing and a 3′ wing.

12. The compound of claim 11, wherein said gap of said gapmer comprises unmodified DNA sugar moieties.

13. The compound of any one of the claims 11 to 12, wherein said 5′ wing of said gapmer comprises at least one abc-DNA nucleoside; and/or wherein said 3′ wing of said gapmer comprises at least one abc-DNA nucleoside.

14. The compound of any one of the claims 11 to 13, wherein said 5′ wing of said gapmer comprises at least one abc-DNA nucleoside, preferably wherein said 5′ wing of said gapmer comprises at least two abc-DNA nucleosides, preferably at least three abc-DNA nucleosides, yet more preferably at least four abc-DNA nucleosides, and again more preferably at least five abc-DNA nucleosides; preferably wherein said abc-DNA nucleosides are contiguous.

15. The compound of any one of the claims 11 to 14, wherein said 3′ wing of said gapmer comprises at least one abc-DNA nucleoside, preferably wherein said 3′ wing of said gapmer comprises at least two abc-DNA nucleosides, preferably at least three abc-DNA nucleosides, yet more preferably at least four abc-DNA nucleosides, and again more preferably at least five abc-DNA nucleosides; preferably wherein said abc-DNA nucleosides are contiguous.

16. The compound of any one of the claims 11 to 15, wherein said 5′ wing of said gapmer comprises at least four abc-DNA nucleosides, and wherein said 3′ wing of said gapmer comprises at least four abc-DNA nucleosides.

17. The compound of any one of the claims 11 to 16, wherein said gap comprises at least five unmodified DNA sugar moieties, preferably at least six unmodified DNA sugar moieties, preferably at least seven unmodified DNA sugar moieties, preferably at least eight unmodified DNA sugar moieties, yet preferably at least nine unmodified DNA nucleosides, yet more preferably at least ten unmodified DNA sugar moieties.

18. The compound of any one of the claims 11 to 17, wherein said gap of said gapmer consists of unmodified DNA sugar moieties; said 5′ wing of said gapmer consists of abc-DNA nucleosides; and said 3′ wing of said gapmer consists of abc-DNA nucleosides.

19. The compound of any one of the claims 11 to 18, wherein at least 20% of the internucleoside linkages within said gap of said gapmer are phosphodiester internucleoside linkages.

20. The compound of any one of the claims 11 to 19, wherein at least 50% of the internucleoside linkages within said gap of said gapmer are phosphodiester internucleoside linkages.

21. The compound of any of the claims 11 to 20, wherein all of the internucleoside linkages within said gap of said gapmer are phosphodiester internucleoside linkages.

22. The compound of any of the claims 11 to 21, wherein at least 70% of the internucleoside linkages within said 5′ wing of said gapmer are phosphodiester internucleoside linkages.

23. The compound of any of the claims 11 to 22, wherein at least 70% of the internucleoside linkages within said 3′ wing of said gapmer are phosphodiester internucleoside linkages.

24. The compound of any one of the claims 11 to 23, wherein 100% of the internucleoside linkages within said 5′ wing and/or said 3′ wing of said gapmer are phosphodiester internucleoside linkages.

25. The compound of any of claims 11 to 24, wherein the internucleoside linkage between said 5′ wing and said gap of said gapmer is a phosphodiester internucleoside linkage.

26. The compound of any of claims 11 to 25, wherein the internucleoside linkage between said 3′ wing and said gap of said gapmer a phosphodiester internucleoside linkage.

27. The compound of any one of claims 11 to 26, wherein said second oligonucleotide comprises at least one unmodified nucleoside.

28. The compound any one of claims 11 to 27, wherein at least one unmodified nucleoside is an unmodified RNA nucleoside.

29. The compound of any one of claims 11 to 28, wherein said second oligonucleotide consists of unmodified RNA nucleosides.

30. The compound of any one of claims 11 to 28, wherein said second oligonucleotide further comprises at least one modified nucleoside, preferably wherein said at least one modified nucleoside is an abcDNA nucleoside or an MOE nucleoside.

31. The compound of claim 30, wherein said at least one modified nucleoside is positioned at the 5′ end and/or the 3′ end of said second oligonucleotide.

32. The compound of any one of claims 11 to 28 or 30 to 31, wherein said second oligonucleotide comprises at most four, preferably at most three, more preferably at most two, more preferably one modified nucleoside and wherein said modified nucleoside is 5′ end and/or the 3′ end of said second oligonucleotide.

33. The compound of any one of claims 11 to 32, wherein at least 80% of the internucleoside linkages within said second oligonucleotide are phosphodiester linkages, preferably wherein at least 90% of the internucleoside linkages with said second oligonucleotide are phosphodiester linkages, more preferably wherein all of the internucleoside linkages within said second oligonucleotide are phosphodiester linkages.

34. The compound of claims 11 to 28 and 30 to 33, wherein at least 70% of the sugar moieties of said second oligonucleotide are unmodified RNA sugar moieties, preferably wherein at least 80%, more preferably at least 90%, more preferably at least 95%, and yet more preferably 100% of the sugar moieties of said second oligonucleotide are unmodified RNA sugar moieties, and wherein preferably at most three of all internucleoside linkages of said second oligonucleotide are phosphorothioate internucleoside linkages, wherein preferably said phosphorothioate internucleoside linkages are positioned at the 5′ end and/or at the 3′ end of said second oligonucleotide.

35. The compound of any one of claims 11 to 29 and 33 to 34, wherein each sugar moiety of said second oligonucleotide is an unmodified RNA sugar moiety, wherein at most three of all internucleoside linkages of said second oligonucleotide are phosphorothioate internucleoside linkages, wherein preferably said phosphorothioate internucleoside linkages are positioned at the 5′-end and/or at the 3′-end of said second oligonucleotide.

36. The compound of any one of the claims 11 to 29 and 33 to 35, wherein each sugar moiety of said second oligonucleotide is an unmodified RNA sugar moiety, and wherein each internucleoside linkage of said second oligonucleotide is a phosphodiester linkage.

37. The compound of any one of claims 11 to 36, wherein said 5′ wing of said gapmer comprises at most five unpaired overhanging nucleotides.

38. The compound of any one of claims 11 to 37, wherein said 3′ wing of said gapmer comprises at most five unpaired overhanging nucleotides.

39. A pharmaceutical composition comprising a compound of any one of the preceding claims and a pharmaceutically acceptable carrier.

40. The compound of any of the claims 1 to 38 or the pharmaceutical composition of claim 39, for use in the prevention or treatment of a disease, wherein preferably said disease is a neuromuscular or musculoskeletal disease, and wherein further preferably the neuromuscular or musculoskeletal disease is selected from the group consisting of Duchenne muscular dystrophy, familial dysautonomia, spinal muscular atrophy, ataxia telangiectasia, congenital disorder of glycosylation, fronto-temporal dementia, Parkinsonism linked to chromosome 17, Niemann-Pick disease type C, neurofibromatosis type 1, neurofibromatosis type 2, megalencephalic leukoencephalopathy with subcortical cysts type 1, Pelizaeus-Merzbacher disease, Pompe disease, myotonic dystrophy type 2 (DM2), and myotonic dystrophy type 1 (DM1).


SEQUENCE LISTING (80 sequences)

SEQ ID NO: 1 - DNA, 11 bp, Artificial Sequence
  Synthetic sequence
  misc_feature: alpha anomeric bicyclo-DNA (abcDNA)
agccgaatgga

SEQ ID NO: 2 - DNA, 15 bp, Artificial Sequence
  Synthetic sequence
  misc_feature: abcDNA
ttggagccgaatgga

SEQ ID NO: 3 - DNA, 18 bp, Artificial Sequence
  Synthetic sequence
  misc_feature: abcDNA
ggtttggagccgaatgga

SEQ ID NO: 4 - DNA, 15 bp, Artificial Sequence
  Synthetic sequence
  misc_feature: abcDNA
  misc_feature (15)..(15): phosphorothioate linkage between A at position 15 and palmitic acid
  misc_binding (15)..(15): palmitic acid
tccattcggctccaa

SEQ ID NO: 5 - DNA, 15 bp, Artificial Sequence
  Synthetic sequence
  misc_feature: abcDNA
tccattcggctccaa

SEQ ID NO: 6 - DNA, 15 bp, Artificial Sequence
  Synthetic sequence
  misc_feature: abcDNA
  misc_feature: Phosphorothioate linkages between all nucleosides.
tccattcggctccaa

SEQ ID NO: 7 - DNA, 11 bp, Artificial Sequence
  Synthetic sequence
  misc_feature: PMO (phosphorodiamidate morpholino oligomer)
aggtaagccga

SEQ ID NO: 8 - DNA, 15 bp, Artificial Sequence
  Synthetic sequence
  misc_feature: PMO
aggtaagccgaggtt

SEQ ID NO: 9 - DNA, 18 bp, Artificial Sequence
  Synthetic sequence
  misc_feature: PMO
aggtaagccgaggtttgg

SEQ ID NO: 10 - DNA, 25 bp, Artificial Sequence
  Synthetic sequence
  misc_feature: PMO
ggccaaacctcggcttacctgaaat

SEQ ID NO: 11 - DNA, 11 bp, Artificial Sequence
  Synthetic sequence
  misc_feature: 2′-MOE oligonucleotide
aggtaagccga

SEQ ID NO: 12 - DNA, 15 bp, Artificial Sequence
  Synthetic sequence
  misc_feature: 2′-MOE oligonucleotide
aggtaagccgaggtt

SEQ ID NO: 13 - DNA, 18 bp, Artificial Sequence
  Synthetic sequence
  misc_feature: 2′-MOE oligonucleotide
aggtaagccgaggtttgg

SEQ ID NO: 14 - DNA, 18 bp, Artificial Sequence
  Synthetic sequence
  misc_feature: 2′-MOE oligonucleotide
  misc_feature: Phosphorothioate linkages between all nucleosides.
ccaaacctcggcttacct

SEQ ID NO: 15 - DNA, 11 bp, Artificial Sequence
  Synthetic sequence
aggtaagccga

SEQ ID NO: 16 - DNA, 15 bp, Artificial Sequence
  Synthetic sequence
aggtaagccgaggtt

SEQ ID NO: 17 - DNA, 18 bp, Artificial Sequence
  Synthetic sequence
aggtaagccgaggtttgg

SEQ ID NO: 18 - DNA, 11 bp, Artificial Sequence
  Synthetic sequence
  misc_feature: FANA oligonucleotide.
aggtaagccga

SEQ ID NO: 19 - DNA, 15 bp, Artificial Sequence
  Synthetic sequence
  misc_feature: FANA oligonucleotide
aggtaagccgaggtt

SEQ ID NO: 20 - DNA, 18 bp, Artificial Sequence
  Synthetic sequence
  misc_feature: FANA oligonucleotide
aggtaagccgaggtttgg

SEQ ID NO: 21 - DNA, 11 bp, Artificial Sequence
  Synthetic sequence
  misc_feature: 2′-OMe oligonucleotide.
aggtaagccga

SEQ ID NO: 22 - DNA, 15 bp, Artificial Sequence
  Synthetic sequence
  misc_feature: 2′-OMe oligonucleotide.
aggtaagccgaggtt

SEQ ID NO: 23 - DNA, 18 bp, Artificial Sequence
  Synthetic sequence
  misc_feature: 2′-OMe oligonucleotide.
aggtaagccgaggtttgg

SEQ ID NO: 24 - DNA, 20 bp, Artificial Sequence
  Synthetic sequence
  misc_feature: 2′-OMe oligonucleotide.
ggccaaaccucggcuuaccu

SEQ ID NO: 25 - DNA, 15 bp, Artificial Sequence
  Synthetic sequence
agguaagccgagguu

SEQ ID NO: 26 - DNA, 18 bp, Artificial Sequence
  Synthetic sequence
agguaagccgagguuugg

SEQ ID NO: 27 - DNA, 25 bp, Artificial Sequence
  Synthetic sequence
auuucagguaagccgagguuuggcc

SEQ ID NO: 28 - DNA, 15 bp, Artificial Sequence
  Synthetic sequence
  misc_feature (14)..(15): phosphorothioate linkage
aggtaagccgaggtt

SEQ ID NO: 29 - DNA, 15 bp, Artificial Sequence
  Synthetic sequence
  misc_feature (13)..(14): phosphorothioate linkage
  misc_feature (14)..(15): phosphorothioate linkage
aggtaagccgaggtt

SEQ ID NO: 30 - DNA, 15 bp, Artificial Sequence
  Synthetic sequence
  misc_feature (1)..(2): phosphorothioate linkage
  misc_feature (13)..(14): phosphorothioate linkage
  misc_feature (14)..(15): phosphorothioate linkage
aggtaagccgaggtt

SEQ ID NO: 31 - DNA, 15 bp, Artificial Sequence
  Synthetic sequence
  misc_feature (15)..(15): 2′-MOE nucleoside
aggtaagccgaggtt

SEQ ID NO: 32 - DNA, 15 bp, Artificial Sequence
  Synthetic sequence
  misc_feature (14)..(15): 2′-MOE nucleosides
aggtaagccgaggtt

SEQ ID NO: 33 - DNA, 15 bp, Artificial Sequence
  Synthetic sequence
  misc_feature (1)..(1): 2′-MOE nucleoside
  misc_feature (14)..(15): 2′-MOE nucleosides
aggtaagccgaggtt

SEQ ID NO: 34 - DNA, 333 bp, Homo sapiens
tttttctttttcttcttttttcctttttgcaaaaacccaaaatattttagctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggtatgagaaaaaatgataaaagttggcagaagtttttctttaaaatgaag

SEQ ID NO: 35 - DNA, 30 bp, Homo sapiens
ccaaactagaaatgccatcttccttgatgt

SEQ ID NO: 36 - DNA, 19 bp, Artificial Sequence
  Synthetic sequence
ctttacggtagaaggaact

SEQ ID NO: 37 - DNA, 18 bp, Artificial Sequence
  Synthetic sequence
ttgatctttacggtagaa

SEQ ID NO: 38 - DNA, 18 bp, Artificial Sequence
  Synthetic sequence
tgatctttacggtagaag

SEQ ID NO: 39 - DNA, 17 bp, Artificial Sequence
  Synthetic sequence
ctttacggtagaaggaa

SEQ ID NO: 40 - DNA, 17 bp, Artificial Sequence
  Synthetic sequence
tttacggtagaaggaac

SEQ ID NO: 41 - DNA, 17 bp, Artificial Sequence
  Synthetic sequence
ttacggtagaaggaact

SEQ ID NO: 42 - DNA, 16 bp, Artificial Sequence
  Synthetic sequence
ctttacggtagaagga

SEQ ID NO: 43 - DNA, 16 bp, Artificial Sequence
  Synthetic sequence
tttacggtagaaggaa

SEQ ID NO: 44 - DNA, 16 bp, Artificial Sequence
  Synthetic sequence
ttacggtagaaggaac

SEQ ID NO: 45 - DNA, 16 bp, Artificial Sequence
  Synthetic sequence
tacggtagaaggaact

SEQ ID NO: 46 - DNA, 15 bp, Artificial Sequence
  Synthetic sequence
ctttacggtagaagg

SEQ ID NO: 47 - DNA, 15 bp, Artificial Sequence
  Synthetic sequence
tttacggtagaagga

SEQ ID NO: 48 - DNA, 15 bp, Artificial Sequence
  Synthetic sequence
ttacggtagaaggaa

SEQ ID NO: 49 - DNA, 15 bp, Artificial Sequence
  Synthetic sequence
tacggtagaaggaac

SEQ ID NO: 50 - DNA, 15 bp, Artificial Sequence
  Synthetic sequence
acggtagaaggaact

SEQ ID NO: 51 - DNA, 14 bp, Artificial Sequence
  Synthetic sequence
ctttacggtagaag

SEQ ID NO: 52 - DNA, 14 bp, Artificial Sequence
  Synthetic sequence
tttacggtagaagg

SEQ ID NO: 53 - DNA, 14 bp, Artificial Sequence
  Synthetic sequence
ttacggtagaagga

SEQ ID NO: 54 - DNA, 14 bp, Artificial Sequence
  Synthetic sequence
tacggtagaaggaa

SEQ ID NO: 55 - DNA, 18 bp, Artificial Sequence
  Synthetic sequence
  misc_feature (1)..(18): a, g, t, c corresponds to abc-DNA modified adenine, guanine, thymine and methylcytosine, respectively.
ctttacggtagaaggaac

SEQ ID NO: 56 - DNA, 18 bp, Artificial Sequence
  Synthetic sequence
  misc_feature (1)..(18): a, g, t, c corresponds to abc-DNA modified adenine, guanine, thymine and methylcytosine, respectively.
tttacggtagaaggaact

SEQ ID NO: 57 - DNA, 312 bp, Homo sapiens
cctccagactagcatttactactatatatttatttttccttttattctagttgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaaggttagtatcaaagatacctttttaaaataaaatactggttacatttgata

SEQ ID NO: 58 - DNA, 49 bp, Homo sapiens
gtacaagaacaccttcagaaccggaggcaacagttgaatgaaatgttaa

SEQ ID NO: 59 - DNA, 276 bp, Homo sapiens
taaaaagacatggggcttcatttttgttttgcctttttggtatcttacaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggtagggcgacagatctaataggaatgaaaacattttagcagactttttaa

SEQ ID NO: 60 - DNA, 47 bp, Homo sapiens
ggtatcttacaggaactccaggatggcattgggcagcggcaaactgt

SEQ ID NO: 61 - DNA, 30 bp, Artificial Sequence
  Synthetic sequence
  misc_feature: PMO (phosphorodiamidate morpholino oligomer)
ctccaacatcaaggaagatggcatttctag

SEQ ID NO: 62 - DNA, 11 bp, Artificial Sequence
  Synthetic sequence
  misc_feature: FANA oligonucleotide.
cuagaaaugcc

SEQ ID NO: 63 - DNA, 16 bp, Artificial Sequence
  Synthetic sequence
  misc_feature (14)..(16): abc DNA
gtcagctgccaatgct

SEQ ID NO: 64 - DNA, 16 bp, Artificial Sequence
  Synthetic sequence
  misc_feature (1)..(3): abc DNA
gtcagctgccaatgct

SEQ ID NO: 65 - DNA, 16 bp, Artificial Sequence
  Synthetic sequence
  misc_feature (1)..(3): abc DNA
  misc_feature (14)..(16): abc DNA
gtcagctgccaatgct

SEQ ID NO: 66 - DNA, 20 bp, Artificial Sequence
  Synthetic sequence
  misc_feature (1)..(5): abc DNA
gggtcagctgccaatgctag

SEQ ID NO: 67 - DNA, 20 bp, Artificial Sequence
  Synthetic sequence
  misc_feature (1)..(5): abc DNA
  misc_feature (6)..(15): phosphorothioate linkage
  misc_feature (16)..(20): abc DNA
gggtcagctgccaatgctag

SEQ ID NO: 68 - DNA, 18 bp, Artificial Sequence
  Synthetic sequence
  misc_feature (1)..(5): abc DNA
  misc_feature (14)..(18): abc DNA
gggtcagctgccaatgct

SEQ ID NO: 69 - DNA, 20 bp, Artificial Sequence
  Synthetic sequence
  misc_feature (1)..(5): abc DNA
  misc_feature (7)..(13): phosphorothioate linkage
  misc_feature (16)..(20): abc DNA
gggtcagctgccaatgctag

SEQ ID NO: 70 - DNA, 20 bp, Artificial Sequence
  Synthetic sequence
  misc_feature (1)..(2): abcDNA
  misc_feature (19)..(20): abcDNA
ctagcauuggcagcugaccc

SEQ ID NO: 71 - DNA, 20 bp, Artificial Sequence
  Synthetic sequence
  misc_feature (1)..(5): abcDNA
  misc_feature (16)..(20): abcDNA
ctagcauuggcagcugaccc

SEQ ID NO: 72 - DNA, 20 bp, Artificial Sequence
  Synthetic sequence
  misc_feature (1)..(5): 2′-MOE nucleoside
  misc_feature (1)..(20): phosphorothioate linkage
  misc_feature (16)..(20): 2′-MOE nucleoside
gggtcagctgccaatgctag

SEQ ID NO: 73 - DNA, 16 bp, Artificial Sequence
  Synthetic sequence
gtcagctgccaatgct

SEQ ID NO: 74 - DNA, 20 bp, Artificial Sequence
  Synthetic sequence
gggtcagctgccaatgctag

SEQ ID NO: 75 - DNA, 16 bp, Artificial Sequence
  Synthetic sequence
agcattggcagctgac

SEQ ID NO: 76 - DNA, 18 bp, Artificial Sequence
  Synthetic sequence
agcattggcagctgaccc

SEQ ID NO: 77 - DNA, 20 bp, Artificial Sequence
  Synthetic sequence
ctagcattggcagctgaccc

SEQ ID NO: 78 - RNA, 16 nt, Artificial Sequence
  Synthetic sequence
agcauuggcagcugac

SEQ ID NO: 79 - RNA, 18 nt, Artificial Sequence
  Synthetic sequence
agcauuggcagcugaccc

SEQ ID NO: 80 - RNA, 20 nt, Artificial Sequence
  Synthetic sequence
cuagcauuggcagcugaccc